(19) United States
(12) Patent Application Publication
(10) Pub. No.: US 2003/0054339 A1
(43) Pub. Date: Mar. 20, 2003

(54) Method for detection of drug-induced mutations in the HIV reverse transcriptase gene

(76) Inventors: Koenraad De Smet, Ghent (BG); Lieven Stuyver, Herzele (BG)

Correspondence Address: Patricia A. Kammerer, HOWREY SIMON ARNOLD & WHITE, LLP, 750 Bering Drive, Houston, TX, 77057-2198, US

(21) Appl. No.: 10/043,875

(22) Filed: Jan. 10, 2002

Related U.S. Application Data
(60) Provisional application No. 60/286,102, filed on Apr. 24, 2001.

(30) Foreign Application Priority Data
Jan. 11, 2001 (EP) 01870005.4
Apr. 20, 2001 (EP) 01870085.6

(51) Int. Cl. C12Q 1/70; C07H 21/02

(52) U.S. Cl. 435/5; 536/23.72

(57) ABSTRACT

The present invention relates to a method for the rapid, reliable and precise detection of drug-induced mutations in the reverse transcriptase gene allowing the simultaneous characterization of a range of codons involved in drug resistance using specific sets of probes optimized to function together in a reverse-hybridization assay. More particularly, the present invention relates to the genetic detection of the following mutations in the HIV RT gene: K103N/R, V106A/I/L, Q151M/L, Y181C/I, M184V/I, Y188L, G190A/S/R and/or T215Y/F/D/S/A. The present invention also relates to probes and primers for such detection, to a diagnostic kit and a LiPA assay. The present invention further relates to HIV RT sequences comprising previously unknown polymorphisms.

### FIELD OF THE INVENTION

The present invention relates to the field of HIV diagnosis. More particularly, the present invention relates to the field of the detection, in an HIV sample, of mutations that are linked to resistance to antiviral drugs used to treat HIV infection.

The present invention relates to a method for the rapid, reliable and precise detection of drug-induced mutations in the HIV reverse transcriptase gene allowing the simultaneous characterization of a range of codons involved in or associated with drug resistance by means of analysis of a set of specific regions within the reverse transcriptase gene, e.g. by using specific sets of probes optimized to function together in a reversed-hybridization assay.

### BACKGROUND OF THE INVENTION

During the treatment of human immunodeficiency virus (HIV) type 1 infected individuals with antiretroviral nucleoside analogues and/or non-nucleoside analogues, emergence of mutations leading to resistance against these drugs has been observed (Hirsch et al, 2000). There are currently three non-nucleoside antiviral drugs, also known as non-nucleoside RT inhibitors (nnRTIs), approved by the FDA for treatment of HIV infections: 11-cyclopropyl-5,11-dihydro-4-methyl-6H-dipyridol(3,2-b:2′,3′-e)diazepin-6-one (NVP, nevirapine), DLV (delavirdine) and EFZ (efavirenz). Mutations associated with resistance can be found at codons 98, 100, 101, 103, 106, 108, 179, 181, 188, 190 and 236 of the HIV reverse transcriptase gene. Most of these have been demonstrated in vitro to confer a significant increase in resistance to one or more nnRTIs. But while most of these can confer resistance, not all tend to appear during treatment. Mutations at codons 103, 106 and 181 are the most common ones. More in particular, mutations at codons 103 and 181 are the most common ones (Kemp et al., 1999). Amongst the nucleoside reverse transcriptase (RT) inhibitors, the nucleoside analogues 3′-azido-2′,3′-dideoxyThymidine (AZT, Zidovudine), abacavir (ABC), 2′,3′-dideoxylnosine (ddI), 2′,3′-dideoxyCytidine (ddC) and (−)-p-L-2′,3′-dideoxy-3′-thioCytidine (3TC), 2′,3′-didehydro-3'deoxyThymidine (D4T) are the most important, since they show a favourable pharmaceutical window of application. All these compounds act in a similar way, namely they serve, after intracellular phosphorylation, as chain terminators of the RT reaction. Upon prolonged treatment with these nucleoside analogues, accumulation of mutations in the viral reverse transcriptase gene (RT) reduce or even annihilate the inhibitory effect of the antivirals. The most important mutations induced by the above compounds and leading to gradually increasing resistance were found at amino acid (aa) positions 41 (M to L), 69 (T to D), 70 (K to R), 74 (L to V), 184 (M to V) and 215 (T to Y or F) (Schinazi et al., 1994). Mutations at aa 65, 67, 75 and 219 have also been reported but these provoked only a minor decrease in sensitivity. More recently, multi-drug-resistant HIV-1 strains were described showing aa changes at codon 62, 69, 75, 77, 116, and 151 (Iversen et al., 1996). In general, these aa changes are the consequence of single point mutations at the first or second codon letter, but in the case of T69D (ACT to GAT), T215Y (ACC to TAC) and T215F (ACC to TTC), two nucleotide mutations are necessary. Whether in these cases the single nucleotide mutation intermediates exist, and if they are of any importance in the mechanism for acquiring resistance is as yet not reported. Third letter variations are in general not leading to an amino acid change, and are therefore seen as natural polymorphisms without further consequence to the resistance pattern.

The treatment regimen for an efficient antiviral treatment is not clear at all. The appearance of one or several of these mutations during antiviral treatment needs to be interpreted in conjunction with the viral load and the amount of CD4 cells. Indeed, it has been shown that the effect of AZT resistance mutations can be suppressed after the appearance of the 3TC induced M184V mutation. The influence of other simultaneously occurring mutations under different combination therapies with respect to the outcome and resistance of the virus has not yet been analysed systematically. In order to get a better insight into the mechanisms of resistance and HIV biology, it is necessary to analyse follow-up patients under antiviral therapy for these mutational events in conjunction with changes of viral titer and CD4 cells.

WO 97/00211 describes a method for detection of drug-induced mutations at positions 41, 67, 69/70, 74/75, 151, 181, 184, 215 and/or 219 in the HIV reverse transcriptase (RT). Many indeterminate results can be noted for codons 184 and 215 based on the probes provided therein. In addition, new mutations have come up during HIV treatment. There is thus an urgent need for an improved method for detection of drug-induced mutations in the HIV RT that takes into account the high variability of the HIV genome.

### AIMS OF THE INVENTION

It is an aim of the present invention to develop a rapid, reliable and precise assay or method for the determination and monitoring of antiviral drug resistance or mutations associated with drug resistance of viruses that contain reverse transcriptase genes and more particularly HIV retroviruses present in a biological sample.

It is another aim of the present invention to develop such an assay or method for the determination and monitoring of mutations associated with HIV drug resistance in a patient.

It is another aim of the present invention to develop such an assay or method for the determination and monitoring of mutations associated with HIV drug resistance in a patient receiving anti-HIV treatment.

It is another aim of the present invention to provide an assay or method that allows the (simultaneous) detection of the different HIV RT gene wild-type and/or mutant codons associated with the antiviral resistance in one single experiment.

It is another aim of the present invention to provide an assay or method that allows to deduce or determine the nucleotide sequence at codons of interest and the corresponding amino acids in the HIV RT gene of HIV retroviruses subject to antiviral therapy. It is another aim of the present invention to amplify the HIV RT gene or part thereof, generate a sequence by any sequencing reaction, analyze the regions prone to mutate under antiviral therapy, and to provide a nucleotide pattern associated with HIV drug resistance.

It is another aim of the present invention to select particular probes able to discriminate wild-type HIV RT sequences from mutated HIV RT sequences, which are associated with resistance to one or more antiviral drugs, such as the nucleoside analogues (nRTIs) ABC, AZT, ddl, ddC, 3TC, D4T and/or others and/or the non-nucleoside reverse transcriptase inhibitors (nnRTIs) NVP, DLV, EFZ and/or others.

It is another aim of the present invention to select particular probes able to discriminate wild-type HIV RT sequences from mutated HIV RT sequences, which are associated with resistance to AZT. It is another aim of the present invention to select particular probes able to discriminate wild-type HIV RT sequences from mutated HIV RT sequences, which are associated with resistance to ABC.

It is another aim of the present invention to select particular probes able to discriminate wild-type HIV RT sequences from mutated HIV RT sequences, which are associated with resistance to ddI.

It is another aim of the present invention to select particular probes able to discriminate wild-type HIV RT sequences from mutated HIV RT sequences, which are associated with resistance to ddC.

It is another aim of the present invention to select particular probes able to discriminate wild-type HIV RT sequences from mutated HIV RT sequences, which are associated with resistance to 3TC.

It is another aim of the present invention to select particular probes able to discriminate wild-type HIV RT sequences from mutated HIV RT sequences, which are * associated with resistance to D4T. It is another aim of the present invention to select particular probes able to * discriminate wild-type HIV RT sequences from mutated HIV RT sequences, which are associated with resistance to NVP.

It is another aim of the present invention to select particular probes able to discriminate wild-type HIV RT sequences from mutated HIV RT sequences, which are associated with resistance to DLV.

It is another aim of the present invention to select particular probes able to discriminate wild-type HIV RT sequences from mutated HIV RT sequences, which are associated with resistance to EFZ.

It is another aim of the present invention to select particular probes able to discriminate wild-type HIV RT sequences from mutated HIV RT sequences, which are associated with resistance to multiple nucleoside analogues (i.e. multidrug resistance).

It is another aim of the present invention to provide an assay or method that allows the (simultaneous) detection of the different HIV RT gene polymorphisms representing wild-type and mutation codons in one single experimental setup.

It is another aim of the present invention to provide an assay or method that allows the detection, more specifically the genetic detection of at least one of the mutations K103N/R, V106A/I/L, Y18IC/I, Q151M/L, M184V/I, Y188L, G19OA/S/R and/or T215Y/F/D/S/A or polymorphisms at or around the respective amino acid positions.

It is another aim of the present invention to provide an assay or method that allows the simultaneous detection, more specifically the genetic detection of at least two of these mutations in one single experiment.

It is another aim of the present invention to provide an assay or method that allows the simultaneous detection, more specifically the genetic detection of at least three of these mutations in one single experiment.

It is another aim of the present invention to provide an assay or method that allows the simultaneous detection, more specifically the genetic detection of at least four of these mutations in one single experiment.

It is another aim of the present invention to provide an assay or method that allows the simultaneous detection, more specifically the genetic detection of at least five of these mutations in one single experiment. It is another aim of the present invention to provide an assay or method that allows the simultaneous detection, more specifically the genetic detection of the mutations K103N/R, V106A/I/L, Y181C/I, Q151M/L, M184V/I, Y188L, G190A/S/R and T215Y/F/D/S/A in one single experiment.

It is another aim of the present invention to select particular probes able to discriminate wild-type HIV RT from mutant HIV RT sequences involving at least one of the amino acid positions 103 (K to N or R), 106 (V to A or I or L), 151 (Q to M or L), 181 (Y to C or I), 184 (M to V or I), 188 (Y to L), 190 (G to A or S or R) and 215 (T to Y or F or D or S or A) of the HIV reverse transcriptase (RT) gene.

It is another aim of the present invention to select particular probes able to detect polymorphisms in and around the above amino acid positions, in addition to the mutations at the above amino acids themselves.

It is another aim of the present invention to select particular probes able to discriminate wild-type from mutant HIV RT sequences involving at least one of the amino acid positions 103, 106, 151, 181, 184 and/or 215 which can be used in a detection method that further detects at least one of the amino acid positions 41 (M to L), 50 (I to T), 67 (D to N), 69 (T to D), 70 (K to R), 74 (L to V), 75 (V to T), 188 (Y to L), 190 (G to A or S or R) and/or 219 (K to Q or E) of the HIV reverse transcriptase (RT) gene. It is another aim of the invention to select particular probes able to detect polymorphisms in and around the above-mentioned positions.

It is another aim of the present invention to select a particular set of probes, able to discriminate wild-type HIV RT sequences from mutated HIV RT sequences, which are associated with resistance to any of the antiviral drugs defined above, for use in a reverse hybridization assay.

It is another aim of the present invention to combine a set of selected probes able to discriminate wild-type HIV RT sequences from mutated HIV RT sequences, which are associated with resistance to antiviral drugs, with another set of selected probes able to identify the HIV isolate, type or subtype present in the biological sample, whereby all probes can be used under the same hybridization and wash-conditions.

It is another aim of the present invention to select primers and/or a set of primers enabling the amplification of the gene fragment(s), which contain mutations known to interfere with or to be associated with resistance to the drugs of interest, or which contain polymorphisms.

It is another aim of the present invention to provide compositions comprising a probe and/or a primer of the present invention.

It is another aim of the present invention to provide isolated polynucleotides and/or fragments thereof for the detection of drug-induced mutations in the HIV RT gene.

It is another aim of the present invention to provide isolated polynucleotides and/or fragments thereof that contain previously unknown polymorphisms in the HIV RT gene.

It is another aim of the present invention to provide isolated polynucleotides and/or fragments thereof for the detection of polymorphisms in the HIV RT gene. The present invention also relates to diagnostic kits comprising the probes of the invention.

The present invention also relates to a line probe assay comprising the probes of the nvention.

All the aims of the present invention have been met by the following specific embodiments.

### FIGURE LEGENDS

FIG. 1. Alignment of 35 selected HIV reverse transcriptase (RT) sequences (SEQ ID NO: 294-328) from plasma samples obtained from HIV-1 infected patients. Target sequences that can be used for probe design are boxed. The RT gene part shown here starts at nucleotide 268. Codons 103, 106, 151, 181, 188 and 190 are shown inbold and are marked with a [symbol] .

FIG. 2. Alignment of 43 selected HIV reverse transcriptase (RT) sequences (SEQ ID NO: 574-616) from plasma samples obtained from HIV-1 infected patients. Target sequences that can be used for probe design are boxed. The RT gene part shown here starts at nucleotide 511. Codons 184 and 215 are shown in bold and are marked with a [symbol] .

FIG. 3. Alignment of 58 selected HIV reverse transcriptase (RT) sequences (SEQ ID NO: 773-830) from plasma samples obtained from HIV-1 infected patients. Target sequences that can be used for probe design are boxed. The RT gene part shown here starts at nucleotide 532. Codons 188 and 190 are shown in bold and are marked with a [symbol] .

FIGS. 4A-F. HIV RT sequences comprising previously unknown polymorphisms around codon 103, from which probes c103w66, c103w107, c103w107b, c103w116, c103w121, c103m22 and c103m26 are derived. Codon 103 is indicated in bold.

FIGS. 5A-E. HIV RT sequences comprising previously unknown polymorphisms around codon 181, from which probes c181w65, c181w65b, c181w65c, c181w69, c181w75, c181w133, c181w133b and c181m26 are derived. Codon 181 is indicated in bold.

FIG. 6. HIV RT sequence comprising previously unknown polymorphisms around codon 184, from which probes c184w85 and c184w85b are derived. Codon 184 is indicated in bold.

FIGS. 7A-C. HIV RT sequence comprising previously unknown polymorphisms around codons 188 and 190, from which probes c188mw76, 188mw76, c188mm77, 188mm77, c188mw86 and 188mw86 are derived. Codons 188 and 190 are indicated in bold.

### TABLES

Table 1. Probes used for the genetic detection of K103N/R and/or V106A/I/L in the HIV reverse transcriptase gene. “-” indicates a nucleotide identical to that of the consensus sequence given above. Non-sense nucleotides introduced at the 3′-end or 5′-end of the probe are represented by small letters.

TABLE 1
Probes used for the genetic detection of K103N/R and/or V106A/I/L
in the HIV reverse transcriptase gene. “-” indicates a nucleotide
identical to that of the consensus sequence given above. Non-sense
nucleotides introduced at the 3′-end or 5′-end of the probe are
represented by small letters.

| | | Consensus Sequence | |
|---|---|---|---|
| | | 102 103 104 105 106 107 108 | |
| Probe | Codon + Amino Acid | A AAG AAA AAA TCA GTA ACA GTA | SEQ ID NO: |
| c103w11 | 103K106V | --- --- --- --- --- --- | 5 |
| c103w43 | 103K106V | -- --- --- --- --- --- -g | 6 |
| c103w46 | 103K106V | - --- --- --- --- --- -g | 7 |
| c103w57 | 103K106V | --- --- --- --- --- --- | 8 |
| c103w62 | 103K106V | --- --- --- --- --- --- | 9 |
| c103w62b | 103K106V | --- --- --- --- --- -- | 865 |
| c103w67 | 103K106V | - --- --- --- --- --- -- | 10 |
| c103w68 | 103K106V | - --- --- --- --- --- --- | 11 |
| c103w69 | 103K106V | -- --- --- --- --- --- -- | 12 |
| c103w70 | 103K106V | -- --- --- --- --- --- --- | 13 |
| c103w71 | 103K106V | --- --- --- --- --- --- -g | 14 |
| c103w72 | 103K106V | --- --- --- --- --- --- -- | 15 |
| c103w28 | 103K106A | -- --- --- --- -C- --- | 16 |
| c103w29 | 103K106A | -- --- --- --- -C- -- | 17 |
| c103w30 | 103K106A | - --- --- --- -C- --- | 18 |
| c103w31 | 103K106A | - --- --- --- -C- --- -g | 19 |
| c103w32 | 103K106A | --- --- --- --- -C- - | 20 |
| c103w64 | 103K106A | --- --- --- --- -C- --- | 21 |
| c103w65 | 103K106A | --- --- --- --- -C- -- | 22 |
| c103w34 | 103K106I | --- --- --- --- A-- --- | 23 |
| c103w35 | 103K106I | ___ ___ ___ ___ A-- --- - | 24 |
| c103w66 | 103K106I | - --- --- --- --- A-- --- | 25 |
| c103w36 | 103K106I | - --- --G --- A-- --- -g | 26 |
| c103w36b | 103K106I | - --- --G --- A-- --- - | 866 |
| c103w37 | 103K106I | --- --- --G --- A-- --- | 27 |
| c103w38 | 103K106I | -- --- --G --- A-- --- | 28 |
| c103w39 | 103K106L | - --- --- --- T-G --- | 29 |
| c103w40 | 103K106L | --- --- --- --- T-G --- | 30 |
| c103w41 | 103K106L | -- --- --- --- T-G --- | 31 |
| c103w42 | 103K106L | -- --- --- --- T-G --- -g | 32 |
| c103w47 | 103K106V | - --G --- --- --- --- | 33 |
| c103w48 | 103K106V | - --G --- --- --- -- | 34 |
| c103w49 | 103K106V | -- --G --- --- --- --- | 35 |
| c103w50 | 103K1O6V | -- --G --- --- --- -- | 36 |
| c103w52 | 103K106V | - --- --- --- --G --- | 37 |
| c103w55 | 103K106V | - --- --G --- --- --- | 38 |
| c103w78 | 103K106V | - --- --G --- --G --- | 39 |
| c103w79 | 103K106V | - --- --G --- --G -- | 40 |
| c103w80 | 103K106V | -- --- --G --- --G -- | 41 |
| c103w81 | 103K106V | -- --- --G --- --G --- | 42 |
| c103w73 | 103K106V | - --- --- --- --- --- A-- | 43 |
| c103w74 | 103K106V | -- --- --- --- --- --- A-- | 44 |
| c103w75 | 103K106V | - --- --- --- --- --- A-- A | 45 |
| c103w76 | 103K103V | - --- --- --- --- --- A-- AC | 46 |
| c103w77 | 103K106V | -- --- --- --- --- --- A-- A | 47 |
| c103w82 | 103K106V | - --- --- --- --T --- | 48 |
| c103w83 | 103K106V | -- --- --- --- --T --- | 49 |
| c103w84 | 103K106V | - --- --- --- --T --- -g | 50 |
| c103w85 | 103K106V | - --- --- --- --T --- -- | 51 |
| c103w91 | 103K106V | -- --- --T --- --- -- | 52 |
| c103w92 | 103K106V | -- --- --T --- --- --- | 53 |
| c103w93 | 103K106V | --- --- --T --- --- -- | 54 |
| c103w94 | 103K106V | --- --- --T --- --- --- | 55 |
| c103w95 | 103K106V | - --- --T --G --- --- | 56 |
| c103w96 | 103K106V | -- --- --T --G --- --- | 57 |
| c103w97 | 103K106V | -- --- --T --G --- -- | 58 |
| c103w98 | 103K106V | - --- --T --G --- -- | 59 |
| c103w99 | 103K106V | - --- -G- --- --- -- | 60 |
| c103w100 | 103K106V | - --- -G---- --- --- | 61 |
| c103w101 | 103K106V | -- --- -G- --- --- -- | 62 |
| c103w102 | 103K106V | -- --- -G- --- --- --- | 63 |
| c103w103 | 103K106V | --- --- G-- --- --- - | 64 |
| c103w104 | 103K106V | - --- G-- --- --- --- | 65 |
| c103w105 | 103K106V | --- G-- --- --- --- -- | 66 |
| c103w106 | 103K106V | -- --- G-- --- --- - | 67 |
| c103w107 | 103K106V | --- --- G-- --G --- Ag | 68 |
| c103w107b | 103K106V | --- --- G-- --G --- A | 867 |
| c103w109 | 103K106V | --- --- G-- --G --- | 69 |
| c103w110 | 103K106V | - --- --- G-- --G -- | 70 |
| c103w111 | 103K106V | --G -G- --- --- --- --- | 71 |
| c103w112 | 103K106V | --- --G -G- --- --- --- | 72 |
| c103w113 | 103K106V | --G -G- --- --- --- | 73 |
| c103w114 | 103K106V | --- --G -G- --- --- -- | 74 |
| c103w115 | 103K106V | -- --G -G- --- --- -- | 75 |
| c103w116 | 103K106V | --- --- --- --- G-- --- | 76 |
| c103w117 | 103K106V | --- --- --- --- --- G-- | 77 |
| c103w118 | 103K106V | --- --- --- --- G-- | 78 |
| c103w119 | 103K106V | --- --- --- --- G-- -- | 79 |
| c103w120 | 103K106V | - --- --- --- --- G-- | 80 |
| c103w12 | 103K106V | --- --C --- --- --- --- | 81 |
| c103m13 | 103N106V | ----C --- --- --- -- | 82 |
| c103m14 | 103N106V | - --C --- --- --- --- | 83 |
| c103m15 | 103N106V | - --C --- --- --- --- -g | 84 |
| c103m16 | 103N106V | -- --C --- --- --- --- | 85 |
| c103m17 | 103N106V | --- --C --- --- --- - | 86 |
| c103m58 | 103N106V | - --C --- --- --- -- | 87 |
| c103m19 | 103N106V | - --T --- --- --- --- -g | 88 |
| c103m59 | 103N106V | -- --T --- --- --- --- | 89 |
| c103m60 | 103N106V | --- --T --- --- --- --- | 90 |
| c103m86 | 103N106V | --- --T --- --- --- --- | 91 |
| c103m87 | 103N106V | -- --T --- --- --- --- -g | 92 |
| c103m88 | 103N106V | --- --T --- --- --- -- | 93 |
| c103m89 | 103N106V | --- --T --- --- --- --- -g | 94 |
| c103m90 | 103N106V | - --- --T --- --- --- --- | 95 |
| c103m22 | 103N106V | -G- --T --- --- --- -- | 96 |
| c103m23 | 103N106V | -G- --T --- --- --- - | 97 |
| c103m24 | 103R106V | - -G- --- --- --- --- | 98 |
| c103m25 | 103R106V | -- -G- --- --- --- --- | 99 |
| c103m26 | 103R106V | -- -G- --- --- --- -- | 100 |
| c103m27 | 103R106V | - -G- --- --- --- -- | 101 |
| | | Consensus Sequence | |
| | | 100 101 102 103 104 105 106 107 108 | |
| Probe | Codon + Amino Acid | TTA AAA AAG AAA AAA TCA GTA ACA GTA | SEQ ID NO: |
| c103w121 | 103K | -- --- --G --- --- -C- | 102 |
| c103w122 | 103K | --- --G --- --- -C- | 103 |
| c103w123 | 103K | -- --- --- --G --- --- | 104 |
| c103w124 | 103K | - --- --- --G --- --- | 105 |
| c103w125 | 103K | --- --G --- --- -C- - | 106 |

TABLE 2
Probes used for the genetic detection of Y181C/I in the HIV
reverse transcriptase gene. “-” indicates a nucleotide identical
to that of the consensus sequence given above. Non-sense
nucleotides introduced at the 3′-end or 5′-end of the probe
are represented by small letters.

| | | Consensus Sequence | |
|---|---|---|---|
| | | 177 178 179 180 181 182 183 184 185 | |
| Probe | Codon+Amino Acid | GAC ATA GTT ATC TAT CAA TAC ATG GAT | SEQ ID NO: |
| c181w1 | 181Y | --- --- --- --- --- | 107 |
| c181w2 | 181Y | - --- --- --- --- --- | 108 |
| c181w3 | 181Y | - --- --- --- --- --- -g | 109 |
| c181w3b | 181Y | g- --- --- --- --- --- - | 868 |
| c181w4 | 181Y | -- --- --- --- --- --- | 110 |
| c181w5 | 181Y | - --- --- --- --- --- -- | 111 |
| c181w6 | 181Y | --- --- -G- --- --- | 112 |
| c181w7 | 181Y | - --- --- -G- --- --- | 113 |
| c181w7b | 181Y | g- --- --- -G- --- --- | 869 |
| c181w8 | 181Y | - --- --- -G- --- --- -g | 114 |
| c181w9 | 181Y | -- --- --- -G- --- --- | 115 |
| c181w10 | 181Y | - --- --- -G- --- --- -- | 116 |
| c181w11 | 181Y | --- --T -G- --- --- | 117 |
| c181w12 | 181Y | - --- --T -G- --- --- | 118 |
| c181w13 | 181Y | G --- --T -G- --- --- 119 | |
| c181w14 | 181Y | -G --- --T -G- --- --- | 120 |
| c181w15 | 181Y | -- --- --T -G- --- --- | 121 |
| c181w16 | 181Y | -G --- --T -G- --- -- | 122 |
| c181w17 | 181Y | --G --- --T -G- --- -- | 123 |
| c181w18 | 181Y | --G --- --T -G- --- --- | 124 |
| c181w19 | 181Y | - --- --- -G- --G --- | 125 |
| c181w20 | 181Y | --- --- -G- --G --- | 126 |
| c181w21 | 181Y | -- --- --- -G- --G --- | 127 |
| c181w22 | 181Y | -- --- --- -G- --G -- | 128 |
| c181w23 | 181Y | --G --- -G- --- --- | 129 |
| c181w24 | 181Y | - --g --- -g- --- --- | 130 |
| c181w25 | 181Y | G --G --- -G- --- --- | 131 |
| c181w26 | 181Y | - --G --- -G- --- -- | 132 |
| c181w27 | 181Y | -- --- --C --- --- -- | 133 |
| c181w28 | 181Y | --- --- --C --- --- -- | 134 |
| c181w29 | 181Y | - --- --- --C --- --- -g | 135 |
| c181w29b | 181Y | g- --- --- --C --- --- - | 870 |
| c181w30 | 181Y | 10 -- --- --- --C --- --- | 136 |
| c181w31 | 181Y | 10 - --C --- --- --- --- | 137 |
| c181w32 | 181Y | 10 -- --C --- --- --- -- | 138 |
| c181w33 | 181Y | --- --C --- --- --- -- | 139 |
| c181w34 | 181Y | -- --C --- --- --- --- | 140 |
| c181w35 | 181Y | --- --T --- --- --- -g | 141 |
| c181w36 | 181Y | --- --T --- --- --- -- | 142 |
| c181w37 | 181Y | --- --T --- --- --- --- | 143 |
| c181w38 | 181Y | -- --T --- --- --- --- - | 144 |
| c181w39 | 181Y | --- --- --T --- --- --- | 145 |
| c181w40 | 181Y | - --- --- --T --- --- -- | 146 |
| c181w41 | 181Y | --- --- --- --- --T -g | 147 |
| c181w44 | 181Y | -- --- --- --- --- --T | 148 |
| c181w46 | 181Y | C --- --- --- --T --- - | 149 |
| c181w47 | 181Y | -C --- --- --- --T --- | 150 |
| c181w49 | 181Y | --- A-- --- --- --- --- | 151 |
| c181w50 | 181Y | -A --- A-- --- --- --- -- | 152 |
| c181w51 | 181Y | -A --- A-- --- --- --- - | 153 |
| c181w52 | 181Y | -A --- A-- --- --- --- G | 154 |
| c181w53 | 181Y | --- --- --A --- --- --- | 155 |
| c181w55 | 181Y | -- --- --A --- --- --- 156 | |
| c181w56 | 181Y | - --- --A --- --- --- -g | 157 |
| c181w57 | 181Y | --G --- --C --- --T | 158 |
| c181w58 | 181Y | --G --- --C --- --T -g | 159 |
| c181w60 | 181Y | - --G --- --C --- --T -g | 160 |
| c181w61 | 181Y | - --G --- --C --- --T | 161 |
| c181w62 | 181Y | - --- --A --- --G --T | 162 |
| c181w63 | 181Y | -- --- --A --- --G --T | 163 |
| c181w64 | 181Y | -- --- --A --- --G --T | 164 |
| c181w65 | 181Y | - --- --A --- --G --T Gg | 165 |
| c181w65b | 181Y | g- --- --A --- --G --T G | 871 |
| c181w65c | 181Y | - --- --A --- --G --T G | 883 |
| c181w66 | 181Y | --- --A --- --G --T Gg | 166 |
| c181w67 | 181Y | --G --- --- --- --G --T | 167 |
| c181w68 | 181Y | --G --- --- --- --G -- | 168 |
| c181w69 | 181Y | -G --- --- --- --G --T | 169 |
| c181w70 | 181Y | G --- --- --- --G --T -g | 170 |
| c181w71 | 181Y | G --- --- --- --G --T | 171 |
| c181w74 | 181Y | -- --- --G --- G-- | 172 |
| c181w75 | 181Y | --- --G --- G-- - | 173 |
| c181w76 | 181Y | A --- --G --- G-- | 174 |
| c181w77 | 181Y | -A --- --G --- G-- | 175 |
| c181w78 | 181Y | A --- --G --- G-- - | 176 |
| c181w79 | 181Y | C --- --- --- --T --- | 177 |
| c181w80 | 181Y | -C --- --- --- --T --- | 178 |
| c181w81 | 181Y | -C --- --- --- --T --- | 179 |
| c181w82 | 181Y | --C --- --- --- --T -- | 180 |
| c181w83 | 181Y | T --- --C --- --- -- | 181 |
| c181w85 | 181Y | --- G-- --- --G --- | 182 |
| c181w86 | 181Y | --C --- --- --G --- | 183 |
| c181w87 | 181Y | -C --- --- --G --- -g | 184 |
| c181w88 | 181Y | -- G-- --- --G --- -g | 185 |
| c181w89 | 181Y | T-- --- --- --- --- - | 186 |
| c181w90 | 181Y | - T-- --- --- --- --- - | 187 |
| c181w91 | 181Y | -- T-- --- --- --- --- - | 188 |
| c181w92 | 181Y | - T-- --- --- --- --- G | 189 |
| c181w93 | 181Y | --- -AC --- --- --- G | 190 |
| c181w94 | 181Y | --- --C --- --- --- G | 191 |
| c181w95 | 181Y | -- --- -AC --- --- --- G | 192 |
| c181w96 | 181Y | -A --- --C --- --- --- G | 193 |
| c181w97 | 181Y | --- --- -AC --- --- -- | 194 |
| c181w98 | 181Y | --A --- --C --- --- -- | 195 |
| c181w99 | 181Y | -- --- -AC --- --- -- | 196 |
| c181w100 | 181Y | -A --- --C --- --- -- | 197 |
| c181w101 | 181Y | --T --- --C --- --- - | 198 |
| c181w102 | 181Y | --- --- --- G-- --- - | 199 |
| c181w103 | 181Y | -T --- --C --- --- -- | 200 |
| c181w104 | 181Y | -- --- --- G-- --- -- | 201 |
| c181w105 | 181Y | -- --- --- --T G-- - | 202 |
| c181w106 | 181Y | --- --- --- --T G-- - | 203 |
| c181w107 | 181Y | - --- --- --- --T G-- - | 204 |
| c181w108 | 181Y | -- --- --- --- --T G-- | 205 |
| c181w109 | 181Y | - --- --- --- --- --T G- | 206 |
| c181w110 | 181Y | --- --- --- --T --- --- | 207 |
| c181w111 | 181Y | -- --- --- --T --- --- - | 208 |
| c181w112 | 181Y | --- --C --- --- --- --T -G | 209 |
| c181w113 | 181Y | -- --C --- --- --- --T -G | 210 |
| c181w114 | 181Y | - --C --- --- --- --T -- | 211 |
| c181w115 | 181Y | --- --- --- --T --- --g | 212 |
| c181w117 | 181Y | -- G-- --- --G --- --- | 213 |
| c181w119 | 181Y | - --- G-- --- --G --- | 214 |
| c181w120 | 181Y | -C --- --- --G --- -- | 215 |
| c181w121 | 181Y | 10 --C --- --- --G --- -- | 216 |
| c181w122 | 181Y | - --C --- --- --G --- | 217 |
| c181w123 | 181Y | --- --C --- --- --G -- | 218 |
| c181w124 | 181Y | -- --- --G --- --- - | 219 |
| c181w125 | 181Y | -- --- --G --- --- --g | 220 |
| c181w126 | 181Y | --- --- --G --- --- - | 221 |
| c181w127 | 181Y | T --- --C --- --- --G - | 222 |
| c181w128 | 181Y | --T --- --C --- --- --G -- | 223 |
| c181w129 | 181Y | - --C --- --- --G --- -g | 224 |
| c181w130 | 181Y | -- --C --- --- --G --- | 225 |
| c181w131 | 181Y | --- --C --- --- --G --- | 226 |
| c181w132 | 181Y | T --- --C --- --- --G --- | 227 |
| c181w133 | 181Y | - --C --- --- --G --- -- | 228 |
| c181w133b | 181Y | g- --C --- --- --G --- -- | 872 |
| c181w134 | 181Y | -- --C --- --- --G --- -- | 229 |
| c181m135 | 181I | - --- --- AT- --- --- - | 230 |
| c181m136 | 181I | --- --- AT- --- --- - | 231 |
| c181m137 | 181I | - A-- --- AT- --- --- - | 232 |
| c181m138 | 181I | g- A-- --- AT- --- --- | 233 |
| c181m139 | 181I | g- --- --- AT- --- --- | 234 |
| c181m140 | 181I | -- A-- --- AT- --- --- | 235 |
| c181m141 | 181I | -- --- --- AT- --- --- | 236 |
| c181m142 | 181I | --- --- --- AT- --- -- | 237 |
| c181m143 | 181I | --- A-- --- AT- --- --- | 238 |
| c181m144 | 181I | --- --- --- AT- --- --- | 239 |
| c181m145 | 181I | G-- A-- --- AT- --- -- | 240 |

TABLE 3
Probes used for the genetic detection of Q151M/L in the HIV
reverse transcriptase gene. “-” indicates a nucleotide
identical to that of the consensus sequence given above.
Non-sense nucleotides introduced at the 3′-end or 5′-end of
the probe are represented by small letters.

| | | Consensus Sequence | |
|---|---|---|---|
| | | 148 149 150 151 152 153 154 155 | |
| Probe | Codon+Amino Acid | GTG CTT CCA CAG GGA TGG AAA GGA | SEQ ID NO: |
| c151w1 | 151Q | --- --- --- --- --- | 241 |
| c151w2 | 151Q | --- --- --- --- -- | 242 |
| c151w3 | 151Q | - --- --- --- --- -- | 243 |
| c151w32 | 151Q | g --- --- --- --- --- | 244 |
| c151w33 | 151Q | -- --- --- --- --- - | 245 |
| c151w34 | 151Q | --- --- --- --- --- - | 246 |
| c151w51 | 151Q | -- --- --- --- --- | 247 |
| c151w52 | 151Q | --- --- --- --- -- | 248 |
| c151w53 | 151Q | -- --- --- --- -- | 249 |
| c151w30 | 151Q | -- --T --- --- --- | 250 |
| c151w31 | 151Q | --- --T --- --- -- | 251 |
| c151w9 | 151Q | -- --C --- --- --- - | 252 |
| c151w10 | 151Q | --- --C --- --- --- C | 253 |
| c151w11 | 151Q | -- --C --- --- --- -- | 254 |
| c151w12 | 151Q | -- --G --- --- --- - | 255 |
| c151w13 | 151Q | --- --G --- --- --- C | 256 |
| c151w14 | 151Q | -- --G --- --- --- -- | 257 |
| c151w6 | 151Q | --- --- --A --- --- | 258 |
| c151w7 | 151Q | --- --- --A --- -- | 259 |
| c151w8 | 151Q | - --- --- --A --- -- | 260 |
| c151w21 | 151Q | --- --- --A --- --- - | 261 |
| c151w22 | 151Q | -- --- --A --- --- - | 262 |
| c151w23 | 151Q | -- --- --A --- --- -- | 263 |
| c151w24 | 151Q | -- --- --A --- --- --- | 264 |
| c151w25 | 151Q | -- --- --- --A --- -- | 265 |
| c151w26 | 151Q | --- --A --- --- --- - | 266 |
| c151w27 | 151Q | g --- --G --- --- --- | 267 |
| c151w28 | 151Q | - --- --A --- --- -- | 268 |
| c151w29 | 151Q | - --- --A --- --- --- | 269 |
| c151w29b | 151Q | g- --- --A --- --- --- | 873 |
| c151m4 | 151M | --- --- AT- --- --- | 270 |
| c151m40 | 151M | -- --- AT- --- --- - | 271 |
| c151m41 | 151M | -- --- AT- --- --- -- | 272 |
| c151m42 | 151M | - --- AT- --- --- -- | 273 |
| c151m43 | 151M | - --- AT- --- --- --- | 274 |
| c151m44 | 151M | --- AT- --- --- -- | 275 |
| c151m45 | 151M | --- AT- --- --- --- | 276 |
| c151m46 | 151M | --- AT- --- --- --- - | 277 |
| c151m50 | 151M | -- AT- --- --- --- - | 278 |
| c151m5 | 151M | --- --- AT- --- -- | 279 |
| c151m19 | 151M | - --- --- AT- --- --- | 280 |
| c151m20 | 151M | --- --- AT- --- --- - | 281 |
| c151m35 | 151M | --- --G AT- --- --- | 282 |
| c151m36 | 151M | --- --G AT- --- -- | 283 |
| c151m37 | 151M | - --- --G AT- --- -- | 284 |
| c151m38 | 151M | -- --G AT- --- --- | 285 |
| c151m39 | 151M | -- --G AT- --- --- - | 286 |
| c151m48 | 151M | -G AT- --- --- --- | 287 |
| c151m49 | 151M | -G AT- --- --- --- - | 288 |
| c151m15 | 151L | --- --- -T- --- --- | 289 |
| c151m16 | 151L | --- --- -T- --- -- | 290 |
| c151m17 | 151L | - --- --- -T- --- -- | 291 |
| c151m18 | 151L | - --- --- -T- --- --- | 292 |
| c151m47 | 151L | -- --- -T- --- --- | 293 |

TABLE 4
Primers used for amplification of the HIV reverse
transcriptase gene or part thereof.

| Primer | Nucleotide Sequence | SEQ ID NO: |
|---|---|---|
| AZT 16-bio | 5′-CCA GT(G/A) AAA TTA AAG CCA GGA ATG GAT GGC CC-3′ | 1 |
| AZT 21-bio | 5′-ATC TGA CTT GCC CAA TT(T/C) AAT TT(T/C) CCC ACT AA-3′ | 2 |
| AZT 35-bio | 5′-AAA CAA TGG CCA TTG ACA GAA G-3′ | 3 |
| AZT 4-bio | 5′-AGT TCA TAA CCC ATC CAA AG-3′ | 4 |

TABLE 5
INNO-LiPA strip set up for simultaneous detection of mutations
at codons 103, 106, 181 and 184 of the HIV reverse transcriptase.
The codon in the HIV reverse transcriptase and the amino acid
detected at said codon are indicated. LiPA strip production and
use are explained in example 2. Several probes can be applied
to a given line.

| | | Codon + Amino |
|---|---|---|
| | | Acid in the HIV |
| Line | Probes | reverse transcriptase |
| 1 | Conjugate control | |
| 2 | Amplification control | |
| 3 | c103w62 or c103w62b, c103w116, | 103K106V |
| | c103w49, c103w115, c103w55, c103w104, | |
| | c103w52, c103w107 or c103w107b, | |
| | c103w92, c103w97 | |
| 4 | c103m26 | 103R106V |
| 5 | c103m14, c103m22 | 103N106V |
| 6 | c103w66, c103w36 or c103w36b | 103K106I |
| 7 | c103w65, c103w121 | 103K106A |
| 8A | c181w3 or c181w3b, c181w38, c181w39, | 181Y |
| | c181w44, c181w53, c181w50, c181w75 | |
| 8B | c181w29 or c181w29b, c181w33, c181w57, | 181Y |
| | c181w65 or c181w65b or c181w65c, | |
| | c181w69, c181w97, c181w133 or | |
| | c181w133b | |
| 9 | c181m7 or c181m7b, c181m14, c181m22, | 181C |
| | c181m26 | |
| 10 | c181m144, c181m140 | 181I |

TABLE 6
INNO-LiPA strip set up for detection of mutations at the multi-drug
resistance codon 151 of the HIV reverse transcriptase. The codon in
the HIV reverse transcriptase and the amino acid detected at said
codon are indicated. LiPA strip production and use are explained in
example 2. Several probes can be applied to a given line.

| | | Codon + Amino |
|---|---|---|
| | | Acid in the HIV |
| Line | Probes | reverse transcriptase |
| 1 | Conjugate control | |
| 2 | Amplification control | |
| 3 | c151w2, c151w51, c151w29 or c151W29b, | 151Q |
| | c151w31, c151w52, c151w53 | |
| 4 | c151m36, c151m48, c151m50 | 151M |

TABLE 7
Summarized results of the analysis of plasma samples for codons
103/106 and 181 via the LiPA assay of the invention. A total of
509 samples were analyzed for codon 103/106, and 464 samples
for codon 181.

| | Codon 103/106 | Codon 181 |
|---|---|---|
| WT | 397 | 349 |
| Mutant | 22 | 19 |
| Mix | 25 | 7 |
| Indeterminate | 36 | 67 |
| No PCR | 29 | 22 |
| (Total) | (509) | (464) |

TABLE 8
Probes used for the genetic detection of M184V/I in the
HIV reverse transcriptase gene. “-” indicates a nucleotide
identical to that of the consensus sequence given above.
Non-sense nucleotides introduced at the 3′-end or 5′-end
of the probe are represented by small letters.

| | | Consensus Sequence | |
|---|---|---|---|
| | | 180 181 182 183 184 185 186 187 188 | |
| Probe | Codon + amino acid | ATC TAT CAA TAC ATG GAT GAT TTG TAT | SEQ ID NO: |
| c184w1 | 184M | - --- --- --- --- --- - | 329 |
| c184w3 | 184M | - --- --- --- --- --- | 330 |
| c184w5 | 184M | --- --- --- --- | 331 |
| c184w7 | 184M | - --- --- --- --- - | 332 |
| c184w9 | 184M | -- --- --- --- --- -- | 333 |
| c184w11 | 184M | - --- --- --- --- --g gg | 334 |
| c184w17 | 184M | - --G --- --- --- --g gg | 335 |
| c184w19as | 184M | - --G --- --- --- - | 336 |
| c184w18as | 184M | -- --- --- --- --- -- | 337 |
| c184w21 | 184M | -- --- --T --- --- - | 338 |
| c184w21b | 184M | -- --- --T --- --- -g | 874 |
| c184w21bis | 184M | g-- --- --T --- --- - | 339 |
| c184w22 | 184M | -- --- --T --- --- -- | 340 |
| c184w23 | 184M | - --- --T --- --- -- | 341 |
| c184w73 | 184M | -C --- --T --- --- - | 342 |
| c184w73bis | 184M | g-C --- --T --- --- - | 343 |
| c184w74 | 184M | --C --- --T --- --- | 344 |
| c184w75 | 184M | --- --T --- --- --g gg | 345 |
| c184w76 | 184M | --- --T --- --- --- -- | 346 |
| c184w77 | 184M | --T --- --- --- --- g | 347 |
| c184w78 | 184M | --T --- --- --- --A --g | 348 |
| c184w79 | 184M | -C --- --T --- --- --g | 349 |
| c184w80 | 184M | --C --- --T --- --- - | 350 |
| c184w69 | 184M | -- --G --T --- --- - | 351 |
| c184w69bis | 184M | g-- --G --T --- --- - | 352 |
| c184w70 | 184M | - --G --T --- --- --g gg | 353 |
| c184w71 | 184M | - --G --T --- --- - | 354 |
| c184w72 | 184M | --- --G --T --- --- - | 355 |
| c184w24 | 184M | - --- --- --- --C --g g | 356 |
| c184w24b | 184M | - --- --- --- --C --g gt | 875 |
| c184w25 | 184M | --- --- --- --C ---gg | 357 |
| c184w26 | 184M | - --- --- --- --C --- gg | 358 |
| c184w34 | 184M | --- --- --- --- --- gg | 359 |
| c184w35 | 184M | --- --- --- --- --- -g | 360 |
| c184w36 | 184M | -C --- --- --- --- | 361 |
| c184w37 | 184M | -C --- --- --- --- - | 362 |
| c184w37bis | 184M | g-C --- --- --- --- - | 363 |
| c184w86 | 184M | --C --- --- --- --- - | 364 |
| c184w86bis | 184M | g --C --- --- --- --- - | 365 |
| c184w38 | 184M | --C --- --- --- --- | 366 |
| c184w44 | 184M | --- --- --- --- --- -- | 367 |
| c184w44bis | 184M | g --- --- --- --- --- -- | 368 |
| c184w45 | 184M | --- --- --- --- --- - | 369 |
| c184w46 | 184M | --- --- --- --- --- --g | 370 |
| c184w81 | 184M | -- --- --- --- A-- --- gg | 371 |
| c184w82 | 184M | --- --- --- --- A-- --- gg | 372 |
| c184w83 | 184M | -- --- --- --- A-- --- -g | 373 |
| c184w84 | 184M | --- --- --- A-- --- -- | 374 |
| c184w85 | 184M | -- --- --- A-- --- --- g | 375 |
| c184w85b | 184M | -- --- --- A-- --- --- | 884 |
| c184m2 | 184V | --- --- G-- --- --- - | 376 |
| c184m4 | 184V | -- --- G-- --- --- - | 377 |
| c184m6 | 184V | -- --- G-- --- --- | 378 |
| c184m8 | 184V | -- --- G-- --- -- | 379 |
| c184m10 | 184V | --- G-- --- --- -- | 380 |
| c184m12 | 184V | --- --- G-- --- --g gg | 381 |
| c184m12bis | 184V | g --- --- G-- --- --g gg | 382 |
| c184m13 | 184I | -- --- --A --- --- gg | 383 |
| c184m14 | 184I | --- --- --A --- --- gg | 384 |
| c184m15 | 184I | --- --- --A --- --- -g | 385 |
| c184m16 | 184V | --- --- G-A --- --- gg | 386 |
| c184m20as | 184V | C- --- --- G-- --- -- | 387 |
| c184m27 | 184I | - --- --- --A --- --- gg | 388 |
| c184m28 | 184I | -- --- --- --A --- --- gg | 389 |
| c184m28b | 184I | g-- --- --- --A --- --- gg | 390 |
| c184m28bis | 184I | -- --G --- G-- --- | 391 |
| c184m29 | 184V | --- --G --- G-- -- | 392 |
| c184m30 | 184V | --- --G --- G-- --- | 393 |
| c184m31 | 184V | - --G --- G-- --- - | 394 |
| c184m32 | 184V | - --G --- G-- --- | 395 |
| c184m33 | 184V | --G --- G-- -- --g gg | 396 |
| c184m39 | 184V | --G --- G-- --- - | 397 |
| c184m40 | 184V | - --G --T G-- --- - | 398 |
| c184m41 | 184V | - --- --T G-- --- --g gg | 399 |
| c184m42 | 184V | --- --T G-- --- -- | 400 |
| c184m42bis | 184V | T --- --T G-- --- -- | 877 |
| c184m42bbis | 184V | --- --T G-- --- --- gg | 401 |
| c184m43 | 184V | --- G-- --- --- --- g | 402 |
| c184m47 | 184V | --- G-- --- --- --- - | 403 |
| c184m48 | 184V | --- G-- --- --- --- --g | 404 |
| c184m49 | 184V | --- G-- --- --- --- --g | 404 |
| c184m50 | 184V | G --- G-- --- --- -g | 405 |
| c184m51 | 184V | G --- G-- --- --- gg | 406 |
| c184m52 | 184V | -G --- G-- --- --- gg | 407 |
| c184m68 | 184V | -G --- G-- --- --g gg | 408 |
| c184m53 | 184V | --G --- G-- --- --- gg | 409 |
| c184m54as | 184V | --- G-- --- --- -- | 410 |
| c184m55 | 184V | - --- G-- --- --- -- | 411 |
| c184m56as | 184V | - --G --- G-- --- - | 412 |
| c184m57as | 184V | g --G --- G-- --- -- | 413 |
| c184m58as | 184V | g --G --- G-- --- - | 414 |
| c184m59 | 184V | --G --T G-- --- --g gg | 415 |
| c184m60as | 184V | G- --G --T G-- --- - | 416 |
| c184m61as | 184V | --G --T G-- --- -- | 417 |
| c184m62 | 184V | --- G-- --- --- -g | 418 |
| c184m62b | 184V | --- G-- --- --- - | 878 |
| c184m62bis | 184V | --- G-- --- --- -- | 419 |
| c184m63as | 184V | G- --G --- G-- --- | 420 |
| c184m64as | 184V | --G --- G-- --- -- | 421 |
| c184m65as | 184V | --G --- G-- --- - | 422 |
| c184m66 | 184V | - --- --G --- G-- | 423 |
| c184m67 | 184V | --- --G --- G-- - | 424 |

TABLE 9
Probes used for the genetic detection of T215Y/F/D/S/A
in the HIV reverse transcriptase gene. “-” indicates a
nucleotide identical to that of the consensus sequence
given above. Non-sense nucleotides introduced at the
3′-end or 5′-end of the probe are represented by
small letters.

| | | | |
|---|---|---|---|
| | | Consensus Sequence | |
| | | 211 212 213 214 215 216 217 218 | |
| Probe | Codon + amino acid | AGG TGG GGA TTT ACC ADA CCA GAC | SEQ ID NO: |

| | | | |
|---|---|---|---|
| c215w8 | 21ST | -- --- --- --- - | 425 |
| c215w24 | 21ST | -- --- --- --- -- | 426 |
| c215w21 | 21ST | -- --- --- --- --- | 427 |
| c215w31 | 21ST | -- --- --- --- --- | 428 |
| c215w19 | 21ST | I-- --- --- --- -- | 429 |
| c215w16 | 21ST | -- --- --- --- --- | 430 |
| c215w9 | 21ST | --- --- --- --- --- | 431 |
| c215w1 | 21ST | - --- --- --- --- --- - | 432 |
| c215w34 | 21ST | - --- --- --- --- --- - | 433 |
| c215w2 | 21ST | - --I I-- --- --- --- -- | 434 |
| c215w25 | 21ST | --- --- --- --- - | 435 |
| c215w29 | 21ST | --- --- --- --- -- | 436 |
| c215w30 | 21ST | --- --- --- --- --- | 437 |
| c215w20 | 21ST | - --- --- I-- --- - | 438 |
| c215w33 | 21ST | - --- --C --- --- - | 439 |
| c215w32 | 21ST | - --G --C --- --- - | 440 |
| c215w65 | 21ST | - --- --G --- --- - | 441 |
| c215w66 | 21ST | --- --G --- --- -- | 442 |
| c215w67 | 21ST | - --- --G --- --- | 443 |
| c215w68 | 21ST | - --- C-G --- --- | 444 |
| c215w69 | 21ST | - --- C-G --- --- - | 445 |
| c215w52 | 21ST | - --- --A --- --- - | 446 |
| c215w53 | 21ST | -- --- --A --- --- | 447 |
| c215w53b | 21ST | c-- --- --A --- --- | 879 |
| c215w53bis | 21ST | g-- --- --A --- --- | 448 |
| c215w54 | 21ST | --- --- --A --- --- | 449 |
| c215w55 | 21ST | -- --G --A --- --- | 450 |
| c2l5w56 | 21ST | - --G --A --- --- - | 451 |
| c215w57 | 214L215T | --- --G --A --- -- | 452 |
| c215w70 | 214L215T | --- --G --A --- --- | 453 |
| c215w71 | 214L215T | --- --G --A --- --- | 454 |
| c215w4 | 214L215T | --- C-- --- --- --- -- | 455 |
| c15w23 | 214L215T | --- --- C-- --- - | 456 |
| c215w10 | 214L215T | -- C-- --- --- --- | 457 |
| c215w27 | 214L215T | --- --- C-- --- -- | 458 |
| c215w35 | 214L215T | --- --- C-- --- --- -- | 459 |
| c215w28 | 214L215T | --- --- C-- --- --- | 460 |
| c215w3 | 214L215T | -- --C A-- --- --- --- -- | 461 |
| c215w50 | 214L215T | -- --G C-- --- --- | 462 |
| c215w50b | 214L215T | c-- --G C-- --- --- | 880 |
| c215w50bis | 214L215T | g-- --G C-- --- --- | 463 |
| c215w51 | 214L215T | - --G C-- --- --- - | 464 |
| c215w5 | 214F215T | --G --- --- --- --- -- | 465 |
| c215w11 | 214F215T | -G --- --- --- --- | 466 |
| c215w36 | 214F215T | --- --G --- --- --- -- | 467 |
| c215w111 | 215T | -- --- --C --- --- | 468 |
| c215w111bis | 215T | g- --- --C --- --- | 469 |
| c215w112 | 214F215T | --- --- --C --- -- | 470 |
| c215w113 | 214F215T | - --- --- --C --- - | 471 |
| c215w114 | 214F215T | --- --- --C --- - | 472 |
| c215w22 | 215T | -- --T --- --- --- | 882 |
| c215w22b | 215T | g-- --T --- --- --- | 881 |
| c215w22bis | 214F215T | G-- --T --- --- --- | 473 |
| c215w37 | 214F215T | - --- --- --T --- --- - | 474 |
| c215w142 | 214F215T | --- --C --T --- -- | 475 |
| c215w143 | 214F215T | --- --C --T --- --- | 476 |
| c215w144 | 214F215T | -- --C --T --- --- - | 477 |
| c215w145 | 214F215T | - --C --T --- --- -- | 478 |
| c215w146 | 214F215T | -- --C --T --- --- -- | 479 |
| c215m147 | 214F215Y | --G --- TT- --- -- | 480 |
| c215m12 | 214F215Y | --- --- TA- --- --- | 481 |
| c215m17 | 214F215Y | --- --- TA- --- -- | 482 |
| c215m38 | 214F215Y | - --- --- TA- --- --- - | 483 |
| c215m40 | 214F215Y | - --- --- TA- --- - | 484 |
| c215m41 | 214F215Y | -- --- --- TA- --- | 485 |
| c215m42as | 214F215Y | -- --- --- TA- --- | 486 |
| c215m43as | 214F215Y | - --- --- TA- --- - | 487 |
| c215m61 | 214F215Y | --- --C TA- --- -- | 488 |
| c215m62 | 214F215Y | -- --C TA- --- -- | 489 |
| c215m63 | 214F215Y | --- --C TA- --- - | 490 |
| c215m64 | 214F215Y | - --- --C TA- --- - | 491 |
| c215m13 | 214F215Y | --- C-- TA- --- -- | 492 |
| c215m14 | 214F215Y | --G --- TA- --- -- | 493 |
| c215m79 | 214F215Y | --G --- TA- --G -- | 494 |
| c215m80 | 214F215Y | -G --- TA- --G -- | 495 |
| c215m81 | 214F215Y | --G --- TA- --G - | 496 |
| c215m82 | 214F215Y | -G --- TA- --G --- | 497 |
| c215m83 | 214F215Y | --- --- TA- --C --- | 498 |
| c215m84 | 214F215Y | -- --- TA- --C --- | 499 |
| c215m85 | 214F215Y | --- --- TA- --C -- | 500 |
| c215m86 | 214F215Y | -- --- TA- --C -- | 501 |
| c215m18 | 214F215F | -- --- TT- --- --- | 502 |
| c215m45 | 214F215F | -- --- TT- --- --- | 503 |
| c215m44 | 214F215F | --- --- TT- --- -- | 504 |
| c215m15 | 214F215F | --- --- TT- --- --- | 505 |
| c215m46 | 214F215F | - --- --- TT- --- - | 506 |
| c215m39 | 214F215F | - --- --- TT- --- --- - | 507 |
| c215m115 | 214F215F | 10 --- --C TT- --- --- | 508 |
| c215m116 | 214F215F | --C TT- --- --- -- | 509 |
| c215m117 | 214F215F | -- --C TT- --- -- | 510 |
| c215m118 | 214F215F | -- --C TT- --- --- | 511 |
| c215m95 | 214L215F | --- C-A TT- --- -- | 512 |
| c215m96 | 214L215F | -- C-A TT- --- --- | 513 |
| c215m97 | 214L215F | -- C-A TT- --- -- | 514 |
| c215m98 | 214L215F | --- C-A TT- --- - | 515 |
| c215m131 | 214L215F | -- C-- TT- --- --- | 516 |
| c215m128 | 214L215F | -- C-- TT- --- --- - | 517 |
| c215m130 | 214L215F | --- C-- TT- --- - | 518 |
| c215m26 | 214L215F | --- C-- TT- --- -- | 519 |
| c215m129 | 214L215F | --- C-- TT- --- -- | 520 |
| c215m127 | 214L215F | --- C-- TT- --- --- | 521 |
| c215m119 | 214F215F | --G --- TT- --- --- | 522 |
| c215m120 | 214F215F | G --- TT- --- --- -- | 523 |
| c215m121 | 214F215F | --G --- TT- --- - | 524 |
| c215m122 | 214F215F | G --- TT- --- --- - | 526 |
| c215m123 | 214F215F | --G --- TT- --G - | 526 |
| c215m124 | 214F215F | G --- TT- --G --- | 527 |
| c215m125 | 214F215F | -G --- TT- --G --- | 528 |
| c215m126 | 214F215F | G --- TT- --G --- -- | 529 |
| c215m75 | 214F215F | - --- --- TAT --- - | 530 |
| c215m76 | 214F215F | --- --- TAT --- -- | 531 |
| c215m77 | 214F215F | --- --- TAT --- --- | 532 |
| c215m78 | 214F215F | - --- --- TAT --- -- | 533 |
| c215m47 | 214F215F | -G --- TAT --- --- | 534 |
| c215m48 | 214F215F | --G --- TAT --- -- | 535 |
| c215m49 | 214F215F | - --G --- TAT --- - | 536 |
| c215m92 | 214F215F | --- TAT --C --- -- | 537 |
| c215m74 | 214F215F | G --- TAT --C --- | 538 |
| c215m73 | 214F215F | -G --- TAT --C -- | 539/ |
| c215m72 | 214F215F | --G --- TAT --C -- | 540 |
| c215m91 | 214F215F | --G --- TAT --C -- | 541 |
| c215m93 | 214F215F | --G --- TAT --C --- | 542 |
| c215m94 | 214F215F | - --G --- TAT --C - | 543 |
| c215m58 | 214F215S | --- C-- T-- --- -- | 544 |
| c215m59 | 214F215S | -- --- C-- T-- --- - | 545 |
| c215m60 | 214F215S | - --- C-- T-- --- - | 546 |
| c215m87 | 214F215F | --- --- TTT --- --- 547 | |
| c215m88 | 214F215F | --- --- TTT --- --- - | 548 |
| c215m89 | 214F215F | - --- --- TTT --- --- | 549 |
| c215m90 | 214F215F | --- --- TTT --- -- | 550 |
| c215m99 | 214F215D | -- --- GA- --- -- | 551 |
| c215m100 | 214F215D | --- --- GA- --- -- | 552 |
| c215m101 | 214F215D | --- --- GA- --- --- | 553 |
| c215m102 | 214F215D | -- --- GA- --- --- | 554 |
| c215m103 | 214F215D | -G --G GA- --- - | 555 |
| c215m104 | 214F215D | - --G --G GA- --- | 556 |
| c215m105 | 214F215D | -- --G --G GA- -- | 557 |
| c215m106 | 214F215D | --G --G GA- --- - | 558 |
| c215m138 | 214L215D | --G --A GA- --- - | 559 |
| c215m139 | 214L215D | --G --A GA- --- -- | 560 |
| c215m140 | 214L215D | --A GA- --- --- -- | 561 |
| c215m141 | 214L215D | -G --A GA- --- --- | 562 |
| c215m107 | 214F215S | --- --- -G- --- --- | 563 |
| c215m108 | 214F215S | - --- -G- --- --- - | 564 |
| c215m109 | 214F215S | --- --- -G- --- -- | 565 |
| c215m110 | 214F215S | --- -G- --- --- -- 566 | |
| c215m132 | 214F215A | --G --- G-- --- - | 567 |
| c215m133 | 214F215A | -G --- G-- --- -- | 568 |
| c215m134 | 214F215A | -G --- G-- --- --- | 569 |
| c215m135 | 214F215A | --G --- G-- --- -- | 570 |
| c215m136 | 214F215A | --- G-- --- --- - | 571 |
| c215m137 | 214F215A | --- G-- --- --- -- | 572 |

TABLE 10
Comparing the original results (WO 97/00211) with those obtained using a prototype of
the improved LiPA RT strip for codon 184. Samples were selected because they had been
“double-blank” in an earlier evaluation

| | Results with improved version | | | | | | |
|---|---|---|---|---|---|---|---|
| | | | | Mutant | Mixture | | |
| Original results | WT | Mutant | WT (weak) | (weak) | WT + mut | Negative | Total |
| WT | 10 | | | | | | |
| Mutant | | 5 | | | | | 5 |
| WT (weak) | 5 | | 2 | | | | 7 |
| Mutant (weak) | | | | | 2 | 1 | 3 |
| Negative | 23 | 12 | 1 | 1 | 4 | 3 | 43 |
| total | 38 | 17 | 3 | 1 | 6 | 4 | 68 |

TABLE 10
Comparing the original results (WO 97/00211) with those obtained using
the improved LiPA for codon 215. Samples were selected because they had been
“double-blank” in an earlier evaluation

| | Results with improved version | | | | | | |
|---|---|---|---|---|---|---|---|
| | | | | Mutant | Mixture | | |
| Original results | WT | Mutant | WT (weak) | (weak) | WT + mut | Negative | Total |
| WT | 5 | | 2 | | | 1 | 8 |
| Mutant | | 1 | | | | 1 | 2 |
| WT (weak) | | | 4 | | | 3 | 7 |
| Mutant (weak) | | | | | | | |
| Negative | 8 | 11 | 2 | | | 27 | 48 |
| total | 13 | 12 | 8 | | | 32 | 65 |

TABLE 12
Probes used for the genetic detection of Y188L and/or
G190A/S/R in the HIV reverse transcriptase gene. “-”
indicates a nucleotide identical to that of the consensus
sequence given above. Non-sense nucleotides introduced
at the 3′-end or 5′-end of the probe are represented by
small letters.

| | | Consensus Sequence | |
|---|---|---|---|
| | | 186 187 188 189 190 191 192 193 | |
| Probe | Codon + Amino Acid | GAT TTG TAT GTA GGA TCT GAC TTA | SEQ ID NO: |
| 188 ww1 | 188Y190G | --- --- --- --- --- | 618 |
| c188 ww1 | 188Y190G | --- --- --- --- --- | 619 |
| 188 ww2 | 188Y190G | - --- --- --- --- --- | 620 |
| 188 ww3 | 188Y190G | -- --- --- --- --- --g | 621 |
| 188 ww4 | 188Y190G | -- --- --- --- --- - | 622 |
| 188 ww5 | 188Y190G | --- --- --- --- --- - | 623 |
| 188 ww6 | 188Y190G | - --- --- --- --- -- | 624 |
| 188 ww7 | 188Y190G | --- --- --- --G --- | 625 |
| 188 ww8 | 188Y190G | - --- --- --- --G --- | 626 |
| 188 ww9 | 188Y190G | -- --- --- --- --G --g | 627 |
| 188 ww10 | 188Y190G | - --- --- --G --- -- | 628 |
| 188 ww11 | 188Y190G | - --- --- --G --- - | 629 |
| 188 ww12 | 188Y190G | - --- --- --G --- - | 630 |
| w188 ww12 | 188Y190G | -- --- --- --G --- | 631 |
| 188 ww13 | 188Y190G | --- --- --- --G --g | 632 |
| 188 ww14 | 188Y190G | -- --- --- --- --G - | 633 |
| 188 ww15 | 188Y190G | --- --- --- --- --- | 634 |
| 188 ww16 | 188Y190G | C --- --- --- --- --- | 635 |
| 188 ww17 | 188Y190G | C --- --- --- --- --g | 636 |
| 188 ww18 | 188Y190G | - --- --- --- --- - | 637 |
| c188 ww18 | 188Y190G | - --- --- --- --- - | 638 |
| 188 ww19 | 188Y190G | - --A --- --- --- --- | 639 |
| 188 ww20 | 188Y190G | - --A --- --- --- --- - | 640 |
| 188 ww21 | 188Y190G | --A --- --- --- --- - | 641 |
| 188 ww22 | 188Y190G | --A --- --- --- --- -- | 642 |
| 188 ww23 | 188Y190G | -A --- --- --- --- -- | 643 |
| 188 ww24 | 188Y190G | -- --A --- --- --- --g | 644 |
| c188 ww24 | 188Y190G | -- --A --- --- --- --g | 645 |
| 188 ww25 | 188Y190G | C-- --- --- --- --- | 646 |
| 188 ww26 | 188Y190G | C-- --- --- --- --g | 647 |
| 188 ww27 | 188Y190G | --A --- --- --- --- - | 648 |
| 188 ww28 | 188Y190G | --A --- --- --- --- -- | 649 |
| 188 ww29 | 188Y190G | -C --A --- --- --- --g | 650 |
| c188 ww29 | 188Y190G | -C --A --- --- --- --g | 651 |
| 188 ww30 | 188Y190G | --- --- --G --- --- | 652 |
| 188 ww31 | 188Y190G | -- --- --G --- --- | 653 |
| c188 ww31 | 188Y190G | -- --- --G --- --- | 654 |
| 188 ww32 | 188Y190G | --- --- --G --- --g | 655 |
| 188 ww33 | 188Y190G | - --- --g --- --- - | 656 |
| 188 ww34 | 188Y190G | --A --- --- --- --A - | 657 |
| 188 ww35 | 188Y190G | -A --- --- --- --A -- | 658 |
| 188 ww36 | 188Y190G | -A --- --- --- --A - | 659 |
| 188 ww37 | 188Y190G | - --A --- --- --- --A | 660 |
| c188 ww37 | 188Y190G | - --A --- --- --- --A | 661 |
| 188 ww38 | 188Y190G | --- --- --- --C --- | 662 |
| 188 ww39 | 188Y190G | --- --- --- --C --g | 663 |
| 188 ww40 | 188Y190G | -- --- --- --C --- | 664 |
| c188 ww40 | 188Y190G | -- --- --- --C --- | 665 |
| 188 ww41 | 188Y190G | -- --- --- --C --- - | 666 |
| 188 ww42 | 188Y190G | - --- --- --- --C --- | 667 |
| 188 ww43 | 188Y190G | - --- --- --- --C --g | 668 |
| 188 ww44 | 188Y190G | --A --- --- --G --- | 669 |
| 188 ww45 | 188Y190G | - --A --- --- --G --- | 670 |
| c188 ww45 | 188Y190G | - --A --- --- --G --- | 671 |
| 188 ww46 | 188Y190G | -- --A --- --- --G --g | 672 |
| 188 ww47 | 188Y190G | --A --- --- --G --- - | 673 |
| 188 ww48 | 188Y190G | --- --- --- --- --A - | 674 |
| 188 ww49 | 188Y190G | -- --- --- --- --A - | 675 |
| 188 ww50 | 188Y190G | - --- --- --- --- --g | 676 |
| 188 ww51 | 188Y190G | --- --- --- --- --A | 677 |
| 188 ww52 | 188Y190G | --- --- --- --G --- | 678 |
| 188 ww53 | 188Y190G | C --- --- --- --G --g | 679 |
| 188 ww54 | 188Y190G | -- --- --- --G --- - | 680 |
| 188 ww55 | 188Y190G | C --- --- --- --G --g | 681 |
| 188 ww56 | 188Y190G | -- --- --- --G --- | 682 |
| 188 ww57 | 188Y190G | -- --- --- --- --C - | 683 |
| 188 ww58 | 188Y190G | -- --- --- --- --C | 684 |
| c188 ww58 | 188Y190G | -- --- --- --- --C | 685 |
| 188 ww59 | 188Y190G | C-- --- --- --- -- | 686 |
| 188 ww60 | 188Y190G | - C-- --- --- --- -- | 687 |
| 188 ww61 | 188Y190G | --- --- --- -C- --- | 688 |
| c188 ww61 | 188Y190G | --- --- --- -C- --- | 689 |
| 188 ww62 | 188Y190G | - --- --- --- -C- --- | 690 |
| 188 ww63 | 188Y190G | -- --- --- --- -C- --g | 691 |
| c188 ww63 | 188Y190G | -- --- --- --- -C- --g | 692 |
| 188 ww64 | 188Y190G | -- --- --- -C- --- - | 693 |
| 188 ww65 | 188Y190G | --- --- --- -C- --- - | 694 |
| 188 ww66 | 188Y190G | - --- --- -C- --- -- | 695 |
| 188 ww67 | 188Y190G | --A --- --- -C- --- - | 696 |
| 188 ww68 | 188Y190G | --A --- --- -C- --- --g | 697 |
| 188 ww69 | 188Y190G | -A --- --- -C- --- --g | 698 |
| 188 ww70 | 188Y190G | - --A --- --- -C- --- - | 699 |
| c188 ww70 | 188Y190G | - --A --- --- -C- --- - | 700 |
| 188 ww71 | 188Y190G | --- --- --G -C- --- | 701 |
| 188 wm72 | 188Y190A | -- --- --G -C- --- | 702 |
| c188 wm72 | 188Y190A | -- --- --G -C- --- | 703 |
| 188 wm73 | 188Y190A | --- --- --G -C- --g | 704 |
| c188 wm73 | 188Y190A | --- --- --G -C- --g | 705 |
| 188 mw74 | 188L190G | -A -TG --- --- --- - | 706 |
| 188 mw75 | 188L190G | --A -TG --- --- --- | 707 |
| 188 mw76 | 188L190G | -A -TG --- --- --- | 708 |
| c188 mw76 | 188L190G | -A -TG --- --- --- | 709 |
| 188 mm77 | 188L190A | -A -TG --- -C- --- - | 710 |
| c188 mm77 | 188L190A | -A -TG --- -C- --- - | 711 |
| 188 mm78 | 188L190A | --A -TG --- -C- --- | 712 |
| 188 mm79 | 188L190A | -A -TG --- -C- --- | 713 |
| c188 mm79 | 188L190A | -A -TG --- -C- --- | 714 |
| 188 wm80 | 188Y190S | --- --- --G A-C --- | 715 |
| 188 wm81 | 188Y190S | -- --- --G A-C --- | 716 |
| 188 wm82 | 188Y190S | --- --- --G A-C --g | 717 |
| c188 wm82 | 188Y190S | --- --- --G A-C --g | 718 |
| 188 mw83 | 188L190G | --- CT- --- --- --g | 719 |
| c188 mw83 | 188L190G | --- CT- --- --- --g | 720 |
| 188 mw84 | 188L190G | --- CT- --- --- --- | 721 |
| 188 mw85 | 188L190G | - --- CT- --- --- --g | 722 |
| 188 mw86 | 188L190G | - --- CT- --- --G - | 723 |
| c188 mw86 | 188L190G | - --- CT- --- --G - | 724 |
| 188 mw87 | 188L190G | --- CT- --- --G --A | 725 |
| 188 mw88 | 188L190G | -- CT- --- --G --A | 726 |
| 188 ww92 | 188Y190G | --- --C --- --- --g | 727 |
| 188 ww93 | 188Y190G | --- --C --- --- --- | 728 |
| 188 ww94 | 188Y190G | -- --C --- --- --- | 729 |
| 188 ww95 | 188Y190G | -C- --- --G --G - | 730 |
| 188 ww96 | 188Y190G | -C- --- --G --G --g | 731 |
| 188 ww97 | 188Y190G | C- --- --G --G --g | 732 |
| 188 ww98 | 188Y190G | C- --- --- --- --- - | 733 |
| 188 ww99 | 188Y190G | - --- --- --- --- - | 734 |
| 188 ww100 | 188Y190G | --- --- A-- --- --- - | 735 |
| 188 ww101 | 188Y190G | - --- --- A-- --- --- | 736 |
| 188 ww102 | 188Y190G | -- --- --- A-- --- --- | 737 |
| 188 ww102 | 188Y190G | - --- --- --- A-- --- | 738 |
| 188 ww104 | 188Y190G | --- --- --- A-- --- A | 739 |
| 188 ww105 | 188Y190G | - --- --- --- A-- --- A | 740 |
| 188 ww106 | 188Y190G | C-- --- --- --- --- | 741 |
| 188 ww107 | 188Y190G | -- --- --- --- - | 742 |
| 188 ww108 | 188Y190G | -- --- --- --- --g | 743 |
| 188 ww109 | 188Y190G | --- --- --- --- - | 744 |
| 188 ww110 | 188Y190G | --- --- --- --- --g | 745 |
| 188 ww111 | 188Y190G | c-- --- --- --- - | 746 |
| 188 ww112 | 188Y190G | - --- --- --- --- | 747 |
| 188 ww113 | 188Y190G | -- --- --- --- --- | 748 |
| 188 ww114 | 188Y190G | 10 -- --A --- --- --- - | 749 |
| 188 ww115 | 188Y190G | --A --- --- --- --- | 750 |
| 188 ww116 | 188Y190G | --A --- --- --- --g | 751 |
| 188 ww117 | 188Y190G | -A --- --- --- --- - | 752 |
| 188 ww118 | 188Y190G | -A --- --- --- --- | 753 |
| 188 ww119 | 188Y190G | -A --- --- --- --g | 754 |
| 188 ww120 | 188Y190G | -A --- --- --- --- --g | 755 |
| 188 wm121 | 188Y190A | -A --- --- -C- --- - | 756 |
| 188 wm122 | 188Y190A | - --A --- --- -C- --- | 757 |
| c188 wm122 | 188Y190A | - --A --- --- -C- --- | 758 |
| 188 wm123 | 188Y190A | -- --A --- --- -C- --- | 758 |
| 188 wm124 | 188Y190A | --A --- --- -C- --- - | 760 |
| 188 wm125 | 188Y190A | --A --- --- -C- --- | 761 |
| 188 wm126 | 188Y190A | --A --- --- -C- --- -cg ga | 762 |
| 188 wm127 | 188Y190A | ---A --- --- -C- --- ccg ga | 763 |
| 188 wm128 | 188Y190S | - --- --- --- A-C --g | 764 |
| c188 wm128 | 188Y190S | - --- --- --- A-C --g | 765 |
| 188 wm129 | 188Y190S | --- --- --- A-C --- | 766 |
| c188 wm129 | 188Y190S | --- --- --- A-C --- | 767 |
| 188 wm130 | 188Y190S | -- --- --- A-C --- - | 768 |
| 188 wm131 | 188Y190S | - --- --- --- A-C --- | 769 |
| 188 ww1b | 188Y190G | --- --- --- --- --- ccg ga | 770 |
| 188 ww12b | 188Y190G | -- --- --- --G --- ccg ga | 771 |
| 188 ww18b | 188Y190G | - --- --- --- --- -cg ga | 772 |

TABLE 13
INNO-LiPA strip set up for detection of mutations at codons 188
and/or 190 of the HIV reverse transcriptase. The codon in the HIV
reverse transcriptase and the amino acid detected at said codon are
indicated. LiPA strip production and use are explained in example 2.
Several probes can be applied to a given line.

| | | Codon + Amino |
|---|---|---|
| | | Acid in the HIV |
| Line | Probes | reverse transcriptase |
| 1 | Conjugate control | |
| 2 | Amplification control | |
| 3A | c188ww1, c188ww24, c188ww29, | 188Y190G |
| | c188ww45 | |
| 3B | c188ww12, c188ww31, c188ww40, | 188Y190G |
| | c188ww58 | |
| 4 | c188wm63, c188wm72, c188wm70, | 188Y190A |
| | c188wm73 | |
| 5 | c188mw76 or 188mw76, c188mw83, | 188L190G |
| | c188mw86 or 188mw86 | |
| 6 | c188mm77 or 188mm77 | 188L190A |
| 7 | c188wm82, c188wm128, c188wm129 | 188Y190S |

TABLE 14

List of probes that comprise previously unknown polymorphisms. These
polymorphisms are indicated in bold. “-”indicates a nucleotide identical to that of the
consensus sequence given above.

| | | | SEQ ID NOs of |
|---|---|---|---|
| | | | HIV RT |
| | | | sequences |
| | | | harbouring the |
| Probe | Codon + amino acid | Consensus Sequence | polymorphisms |
| | | 102 103 104 105 106 107 108 | |
| | | A AAG AAA AAA TCA GTA ACA GTA | |
| c103w66 | 103K106I | - --- --- --- --- A-- --- | 833, 307 |
| c103w107b | 103K106V | --- --- G-- --G --- A | 835, 317 |
| c103w116 | 103K106V | --- --- --- --- G-- --- | 837, 319 |
| | | 100 101 102 103 104 105 106 107 108 | |
| | | TTA AAA AAG AAA AAA TCA GTA ACA GTA | |
| c103w121 | 103K | -- --- --G --- --- -C- | 839, 321 |
| | | 102 103 104 105 106 107 108 | |
| | | AAG AAA AAA TCA GTA ACA GTA | |
| c103m22 | 103N106V | -G- --T --- --- --- -- | 841, 304 |
| c103m26 | 103R106V | -- -G- --- --- --- -- | 843, 322 |
| | | 177 178 179 180 181 182 183 184 185 | |
| | | GAC ATA GTT ATC TAT CAA TAC ATG GAT | |
| c181w65c | 181Y | - --- --A --- --G --T G | 845, 309 |
| c181w69 | 181Y | -G --- --- --- --G --T | 847, 312 |
| c181w75 | 181Y | --- -- G --- G-- - | 849, 300 |
| c181w133 | 181Y | - --C --- --- --G --- -- | 851, 297 |
| c181m26 | 181C | - --G --- -G- --- -- | 853, 321 |
| | | 180 181 182 183 184 185 186 187 188 | |
| | | ATC TAT CAA TAC ATG GAT GAT TTG TAT | |
| c184w85b | 184M | -- --- --- A-- --- --- | 855 |
| | | 186 187 188 189 190 191 192 193 | |
| | | GAT TTG TAT GTA GGA TCT GAC TTA | |
| 188mw76 | 188L190G | -A -TG --- --- --- | 857, 858, 859, |
| | | | 860 |
| c188mw76 | 188L190G | -A -TG --- --- --- | 857, 858, 859, |
| | | | 860 |
| 188mm77 | 188L190A | -A -TG --- -C- --- - | 862, 326 |
| c188mm77 | 188L190A | -A -TG --- -C- --- - | 862, 326 |
| 188mw86 | 188L190G | - --- CT- --- --G - | 864 |
| c188mw86 | 188L190G | - --- CT- --- --G - | 864 |

### DETAILED DESCRIPTION OF THE INVENTION

The present invention relates to a method for the detection and/or monitoring of mutations associated with anti-HIV drug resistance in a patient by genetic detection of at least one of the mutations K103N/R, V106A/I/L, Q151M/L, Y181C/I, M184V/I, Y188L, G190A/S/R and/or T215Y/F/D/S/A in the reverse transcriptase (RT) of HIV strains, present in a biological sample of said patient, comprising the following steps:

(i) if needed, release, isolation and/or concentration of the polynucleic acids present in said biological sample;

(ii) if needed, amplification of the HIV reverse transcriptase gene or a part thereof in said biological sample with at least one suitable primer pair;

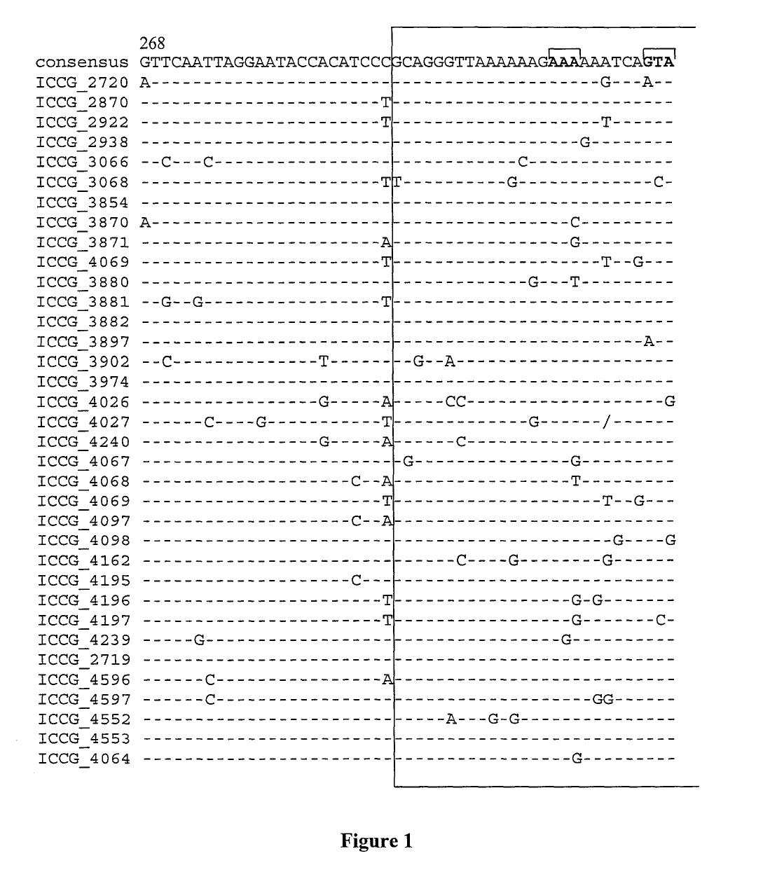

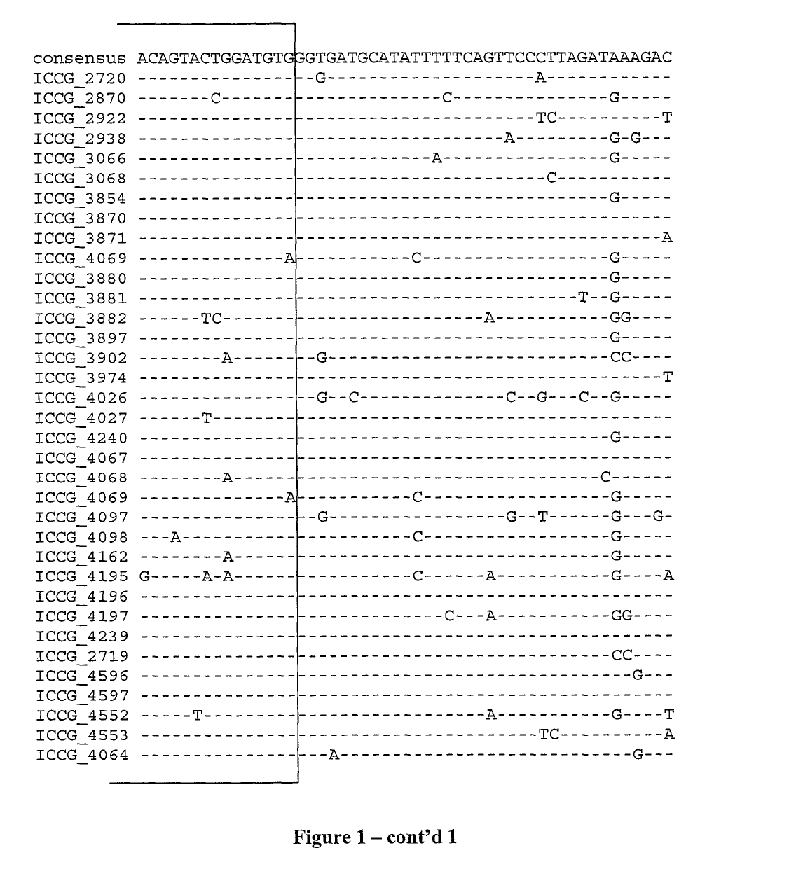

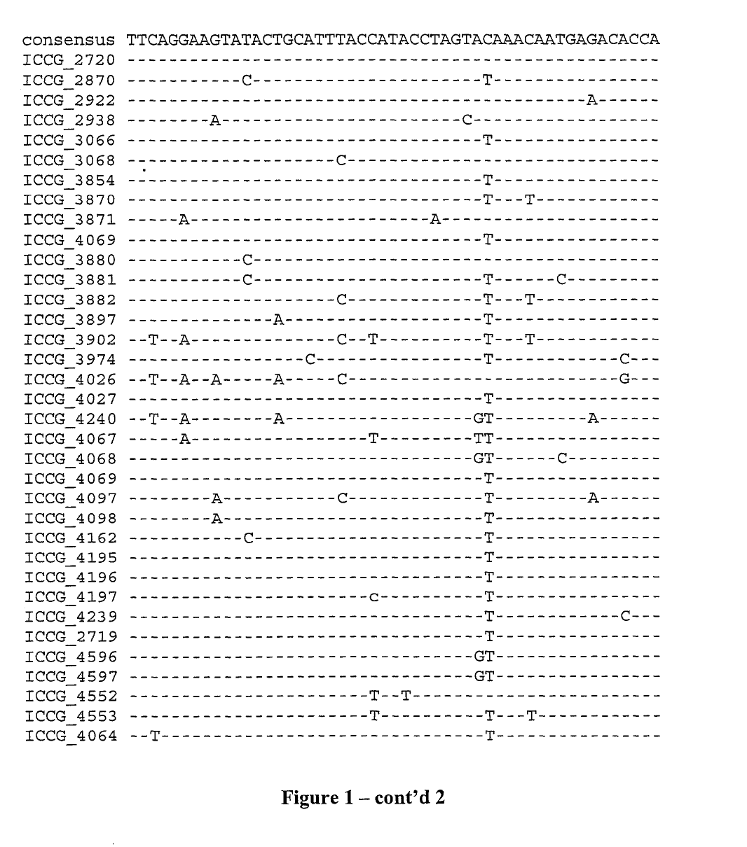

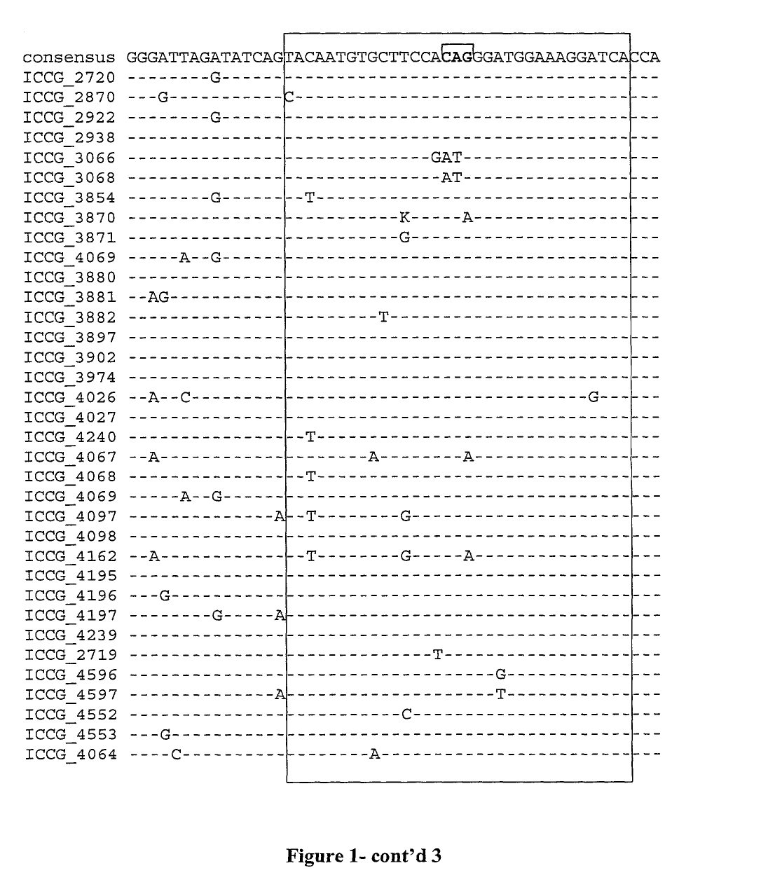

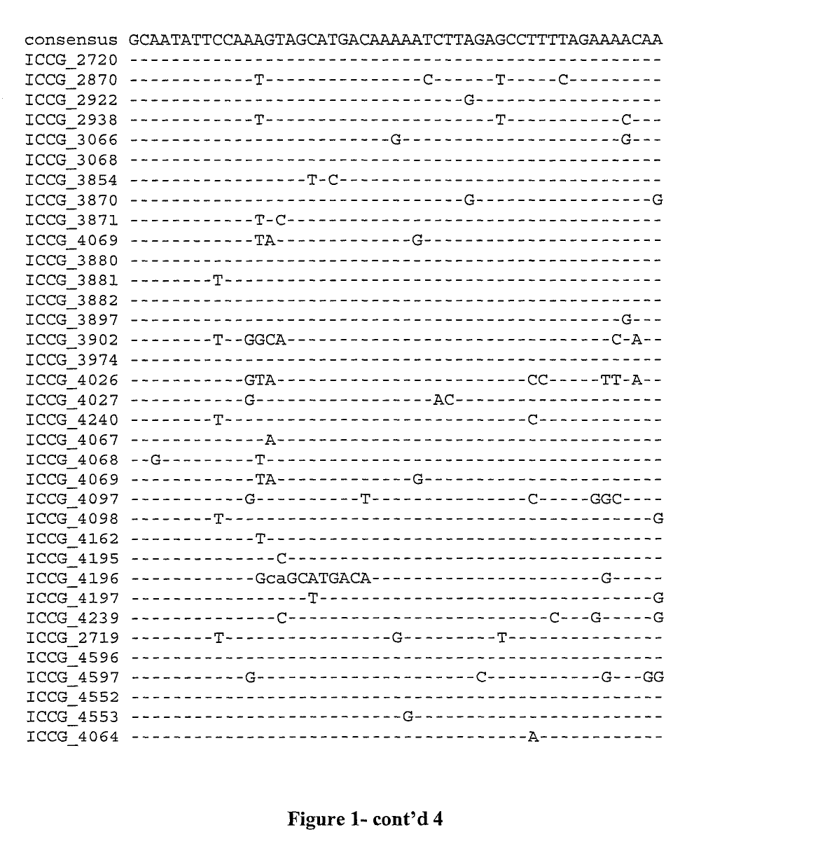

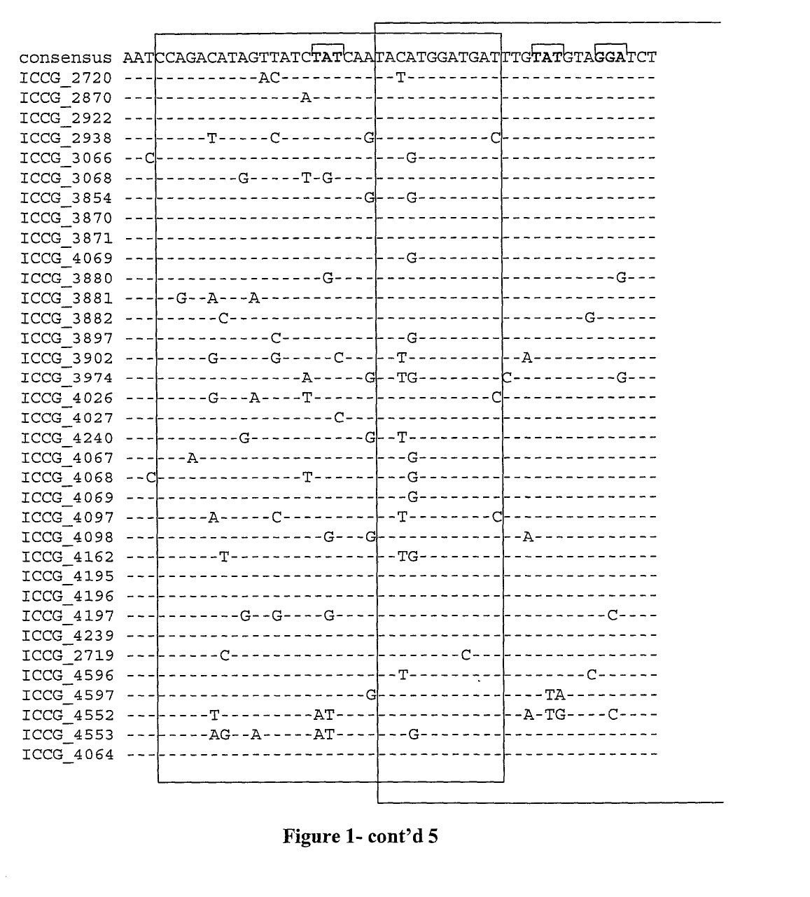

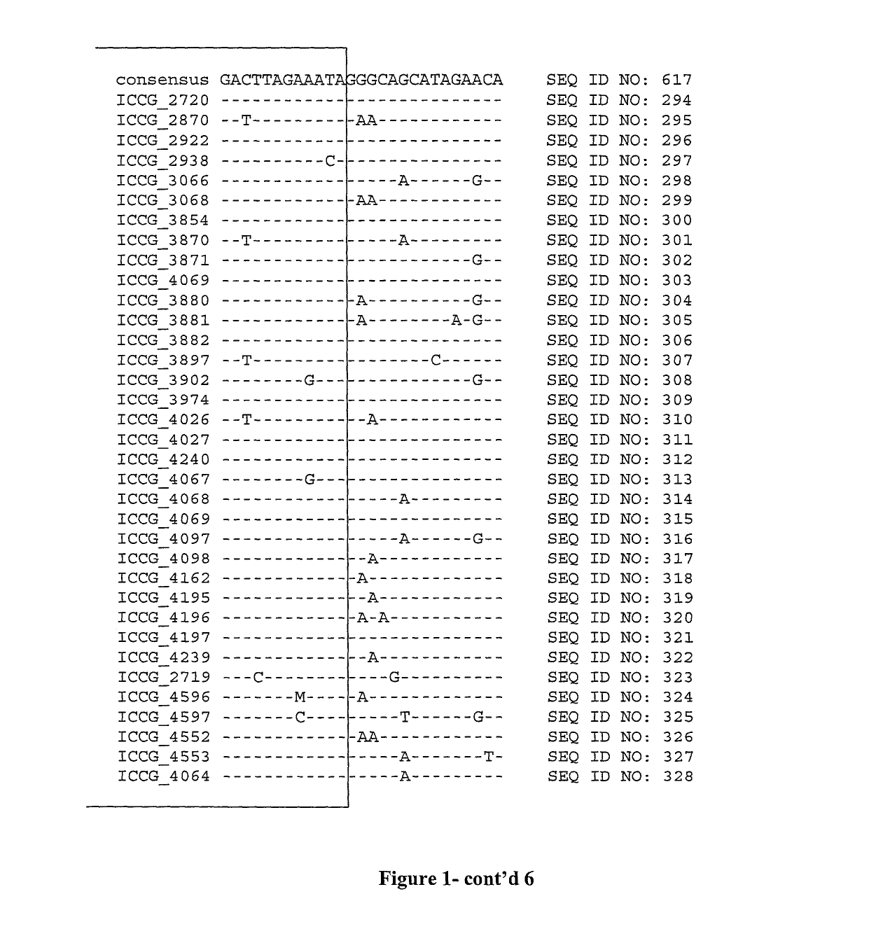

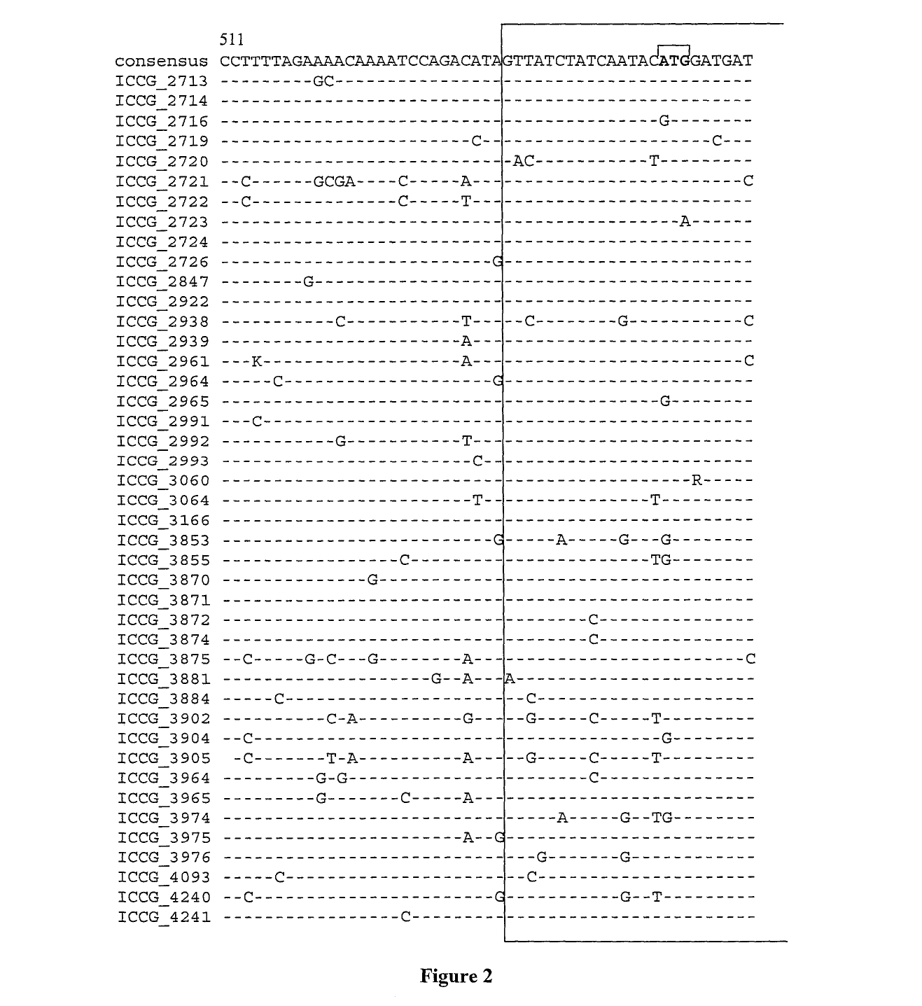

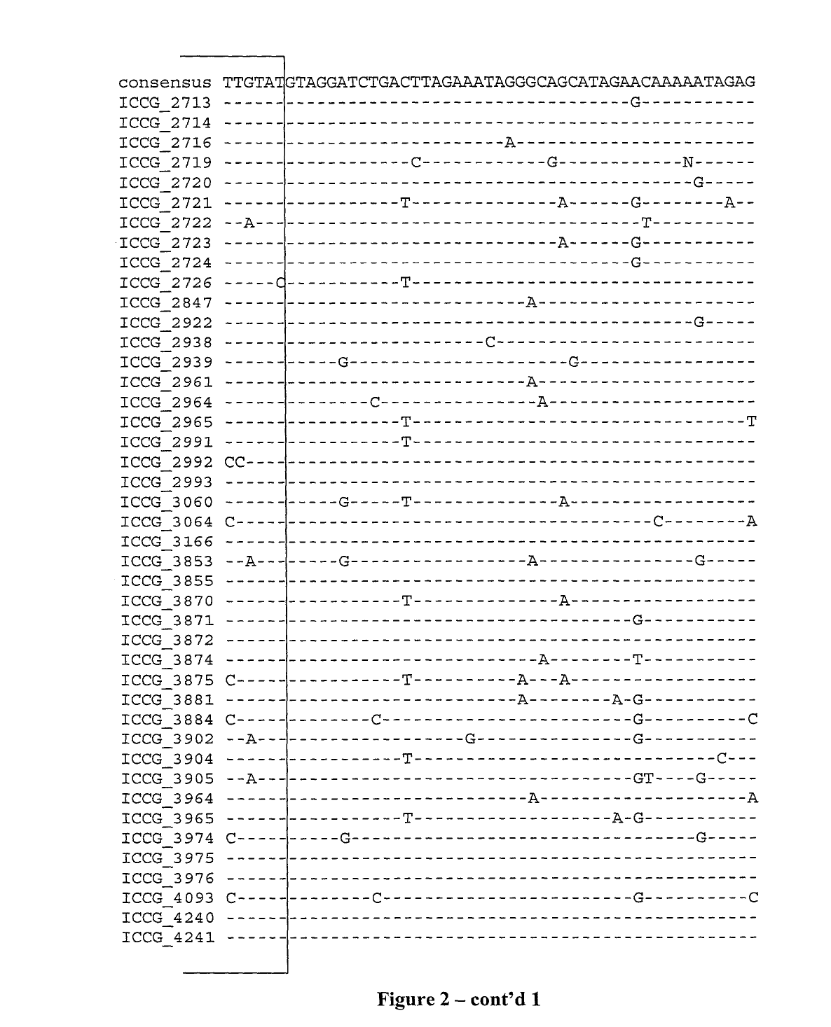

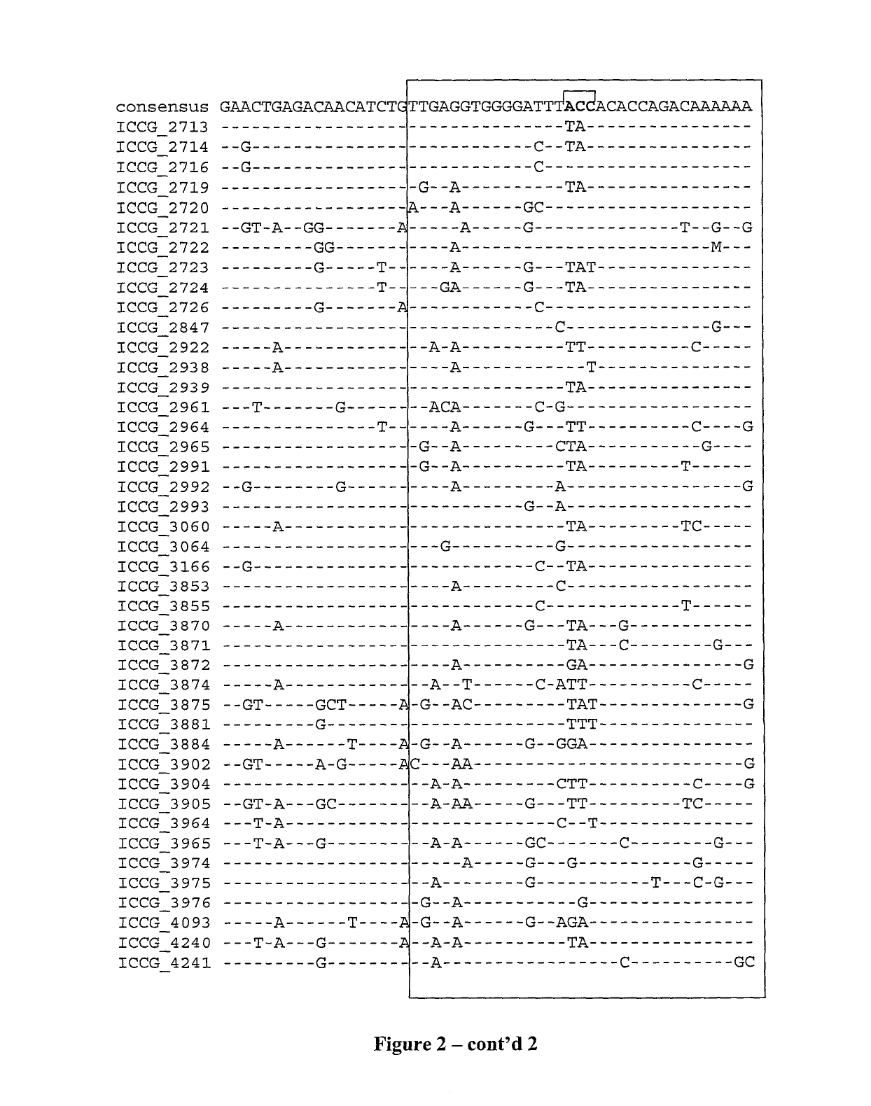

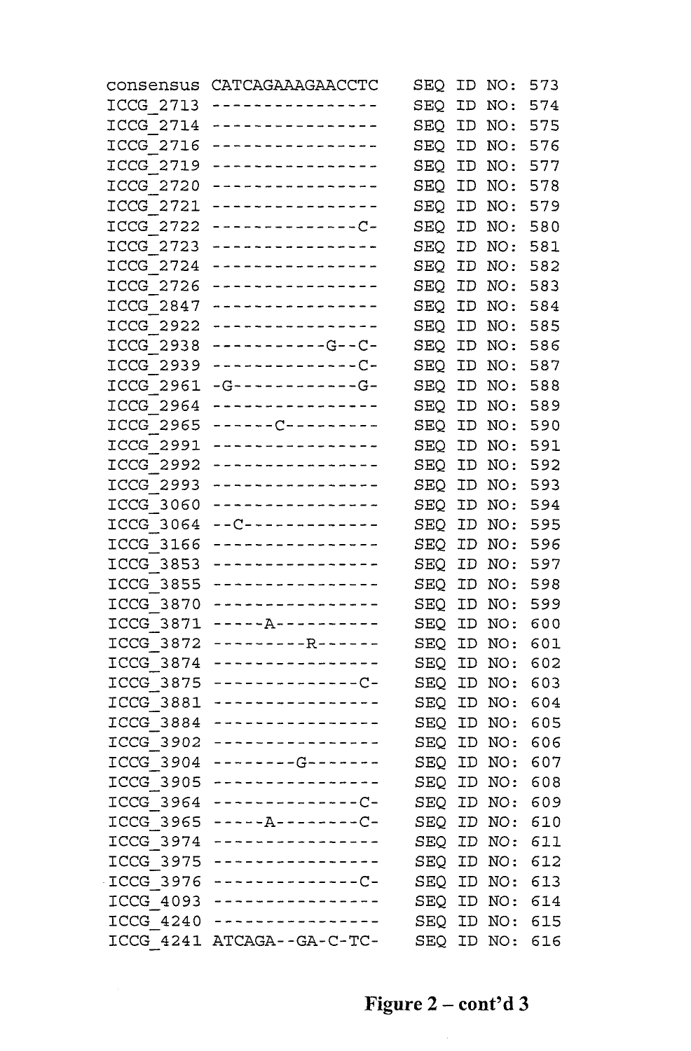

(iii) hybridization of the polynucleic acids in the sample, possibly released, isolated, concentrated and/or amplified via steps (i) and/or (ii), with at least one probe capable of specifically hybridizing with a target sequence in the HIV reverse transcriptase gene or its complement, or specifically hybridizing with a sequence wherein T in said target sequence is replaced by U, said target sequence being selected from the target sequences shown in FIGS. 1, 2 and/or 3;

(iv) detection of the hybrids formed in step (iii);

(v) inference, from the hybridization signal obtained in step (iv), of the presence or absence of the K103N/R, V106A/I/L, Q151M/L, Y18IC/I, M184V/I, Y188L, G190A/S/R and/or T215Y/F/D/S/A mutation in the HIV reverse transcriptase, and of possible anti-HIV drug resistance of the HIV strains present in said biological sample.

Drug resistance in HIV is associated with the presence of at least one or an accumulation of several mutations. Specific mutations are associated with specific drugs or drug classes. The term “mutations associated with drug resistance” refers to mutations in certain codons that arise during antiviral therapy and that may be associated with resistance of the strain to the drug or drug class used. Resistance in HIV or other viruses can be determined either by phenotypic assays or by identifying mutations, and correlating these with a resistance pattern according to certain algorithms. It should be clear that virtual phenotyping, which is based on the analysis of a link between a sequence derived from a sample and a phenotype database, is considered to belong to the latter category.

The method for the detection and/or monitoring of mutations associated with anti-HIV drug resistance, which is described above, allows the genetic detection of mutations in at least one of the codons 103, 106, 151, 181, 184, 188, 190 and/or 215 of the HIV RT gene. The isolation and characterization of a large number of HIV-1 RT gene sequences has allowed the inventors to develop a reference panel of target sequences, which can be used to construct a very specific and very sensitive hybridization assay for detection of the above mentioned mutations.

The mutation K103N/R designates that the genetic code for lysine (K) in codon 103 of the HIV RT gene is substituted by the genetic code for asparagine (N) or the genetic code for arginine (R). The mutation V106A/I/L designates that the genetic code for valine (V) is substituted by the genetic code for alanine (A), the genetic code for isoleucine (I) or the genetic code for leucine (L) in codon 106 of the HIV RT gene. The mutation Y181C/I designates that the genetic code for tyrosine (Y) is substituted by the genetic code for cysteine (C) or the genetic code for isoleucine (I) in codon 181 of the HIV RT gene. The mutation M184V/I designates that the genetic code for methionine (M) is substituted by the genetic code for valine (V) or the genetic code for isoleucine (I) in codon 184 of the HIV RT gene. The mutation Y188L designates that the genetic code for tyrosine (Y) is substituted by the genetic code for leucine (L) in codon 188 of the HIV RT gene. The mutation G190A/S/R designates that the genetic code for glycine (G) is substituted by the genetic code for alanine (A), the genetic code for serine (S) or the genetic code for arginine (R) in codon 190 of the HIV RT gene. The mutation T215Y/F/D/S/A designates that the genetic code for threonine (T) is substituted by the genetic code for tyrosine (Y), the genetic code for phenylalanine (F), the genetic code for aspartate (D), the genetic code for serine (S) or the genetic code for alanine (A) in codon 215 of the HIV RT gene. The mutation Q151M/L designates that the genetic code for glutamine (Q) is substituted by the genetic code for methionine (M) or the genetic code for leucine (L) in codon 151 of the HIV RT gene. A mutation at codon 151 is associated with cross-resistance to several drugs of the class of nucleoside analogue reverse transcriptase inhibitors (nRTIs).

The term “detection” or “genetic detection of a mutation” as used in the present invention means that a mutation in an amino acid sequence is detected by determination of the corresponding nucleic acid sequence.

In one embodiment of the present invention, the mutations in codons 103, 106, 151, 181, 184, 188, 190 and/or 215 of the HIV RT gene are detected by hybridization of the nucleic acids present in the patient's biological sample, with one or more probes that are capable of specifically hybridizing with a target sequence in the HIV RT gene as shown in FIGS. 1, 2 and/or 3. The term “to hybridize specifically” means that said probe forms a duplex with part of its target sequence or with the entire target sequence under the experimental conditions used. Under such specifically selected conditions, said specific probe does not form a duplex with other sequences of the polynucleic acids present in the sample to be analyzed.

The term “target sequence” of a probe, according to the present invention, is a sequence within the HIV RT gene or any polynucleic acid derivative thereof that comprises a mutated or a wild type nucleic acid sequence of the codon encoding amino acid 103, 106, 151, 181, 184, 188, 190 and/or 215 of the HIV RT gene and to which the probe is completely complementary or partially complementary (i.e. with up to 20%, more preferably 15%, more preferably 10% or most preferably 5% mismatches). It is to be understood that the complement of said target sequence is also a suitable target sequence in some cases. It should be understood that probes that are designed to specifically hybridize to a target sequence of a nucleic acid, may fall within said target sequence or may to a large extent overlap with said target sequence (i.e. form a duplex with nucleotides outside as well as within said target sequence). Target sequences used in the method of the present invention for the design of the probes are indicated in FIGS. 1, 2 and/or 3. These target sequences comprise at least the codon sequence to be detected. In addition, these target sequences comprise at least 1, 2, 3, more preferably at least 4, 5, 6, most preferably at least 7, 8, 9, 10, 11, 12, 13, 14, 15 or more nucleotides upstream and/or downstream of said codon sequence.

In a particular embodiment of the invention, the target sequence spans at least nucleotide positions 304 to 315, 303 to 316, 302 to 317, 307 to 318, 306 to 319, 305 to 320, 445 to 453, 444 to 454, 443 to 455, 449 to 455, 448 to 456, 447 to 457, 535 to 543, 534 to 5 544, 533 to 545, 538 to 546, 537 to 547, 536 to 548, 541 to 549, 540 to 550, 539 to 551, 541 to 566, 544 to 555, 543 to 556, 542 to 557, 550 to 561, 549 to 562, 548 to 563, 562 to 570, 561 to 571, 560 to 572, 637 to 645, 636 to 646, 635 to 647, 640 to 648, 639 to 649, 638 to 650, 643 to 651, 642 to 652, 641 to 653 and/or 634 to 656 of the HIV reverse transcriptase gene.

The term “complementary” or “complement” as used herein means that the sequence of the single-stranded probe is exactly the (inverse) complement of the sequence of the single-stranded target, with the target being defined as the sequence where the mutation to be detected is located.

The term “probe” refers to a single-stranded sequence-specific oligonucleotide that has a sequence that is complementary to the target sequence of the HIV reverse transcriptase gene. Preferably, the probe is about 5 to 50 nucleotides long, more preferably from about 10 to nucleotides. Particularly preferred lengths of probes include 10, 11, 12, 13, 14, 15, 16, 17, 18, 19, 20, 21, 22, 23, 24 or 25 nucleotides. The nucleotides used in the probes of the present invention may be ribonucleotides, deoxyribonucleotides and modified nucleotides such as inosine, or nucleotides containing modified groups that do not change their specificity but may alter their hybridization characteristics. For instance, oligonucleotides in which one or more purine residues are substituted by pyrazolo[3,4-d]pyrimidine base analogues have higher melting temperatures than unsubstituted oligonucleotides of identical sequence (U.S. Pat. No. 6,127,121). With probes modified as taught in United States Patent 6,127,121, higher hybridization signals can be obtained and mismatch discrimination is enhanced. Consequently, the necessary probe length will decrease and a smaller amount of probes will suffice to cover a highly variable target region. Non-sense nucleotides can be introduced to at the 5′-end or 3′-end of the probe to control better the hybridization pattern of the probe.

The oligonucleotides of the invention, which are generally referred to as probes throughout the specification, are sequence specific oligonucleotides and can as such be used as sequence specific primers in methods such as PCR-SSP (Olerup and Zetterquist, 1991).

Probe sequences are represented throughout the specification as single-stranded DNA oligonucleotides from the 5′ to the 3′ end. It is obvious to the man skilled in the art that any of the below-specified probes can be used as such, or in their complementary form, or in their RNA form (wherein T is replaced by U).

Since the current application requires the detection of single base pair mismatches, stringent conditions for hybridization of probes are required. However, it should be noted that, since the central part of the probe is essential for its hybridization characteristics, possible deviations of the probe sequence versus the target sequence might be allowable near the extremities of the probe when longer probe sequences are used. When other hybridization conditions would be preferred, probes may be adapted accordingly by adding or deleting one or more nucleotides at their extremities. It should be understood that these concomitant adaptations should give rise to the same results, namely that the probes still hybridize specifically to their respective target sequences. Such adaptations may also be necessary if the amplified material is RNA and not DNA as is the case in the NASBA system. Said deviations and variations, which may be conceived from the common knowledge in the art, should however always be evaluated experimentally, in order to check if they result in equivalent hybridization characteristics.

The probes according to the invention can be prepared by cloning recombinant plasmids containing inserts including the corresponding nucleotide sequence, if need be by excision of the latter from the cloned plasmids by use of the adequate nucleases and recovering them, e.g. by fractionation according to molecular weight. The probes according to the present invention can also be synthesized chemically, for instance by the conventional phospho-triester method.

In order to enhance binding to a solid support, probes can be provided with a poly(dt) tail or the like. Said tail can be added enzymatically or chemically or by any other method known in the art.

Examples of probes of the invention are represented in Tables 1 to 3, 8 to 9 and 12. These probes of the invention are designed to attain optimal performance under identical hybridization conditions so that they can be used in sets of at least 2 probes for simultaneous hybridization; this highly increases the usefulness of these probes and results in a significant gain in time and labour. Evidently, when other hybridization conditions would be preferred, probes should be adapted accordingly by adding or deleting a number of nucleotides at their extremities. It should be understood that these concomitant adaptations should give rise to essentially the same result, namely that the respective probes still hybridize specifically with the defined target. Such adaptations might also be necessary if the amplified material is RNA instead of DNA as in the case for the NASBA (nucleic acid sequence-based amplification) system.

In a specific embodiment, the probe used in a method of the invention is selected from Tables 1, 2, 3, 8, 9 and/or 12 wherein:

the probes specifically hybridizing to the K103N/R target sequences are selected from the following list: SEQ ID NO: 5 to SEQ ID NO: 106 and SEQ ID NO: 865 to SEQ ID NO: 867;

the probes specifically hybridizing to the V106A/I/L target sequences are selected from the following list: SEQ ID NO: 5 to SEQ ID NO: 101;

the probes specifically hybridizing to the Y181C/I target sequences are selected from the following list: SEQ ID NO: 107 to SEQ ID NO: 240, SEQ ID NO: 868 to SEQ ID NO: 872 and SEQ ID NO: 883;

the probes specifically hybridizing to the Q151 M/L target sequences are selected from the following list: SEQ ID NO: 241 to SEQ ID NO: 293 and SEQ ID NO: 873;

the probes specifically hybridizing to the MI 84V/I target sequences are selected from the following list: SEQ ID NO: 329 to SEQ ID NO: 424, SEQ ID N02: 874 to SEQ ID NO: 878 and SEQ ID NO: 884;

the probes specifically hybridizing to the Y188L target sequences are selected from the following list: SEQ ID NO: 618 to SEQ ID NO: 772;

the probes specifically hybridizing to the G19OA/S/R target sequences are selected from the following list: SEQ ID NO: 618 to SEQ ID NO: 772;

the probes specifically hybridizing to the T215Y/F/D/S/A target sequences are selected from the following list: SEQ ID NO: 425 to SEQ ID NO: 572 and SEQ ID NO2: 879 to SEQ ID NO: 882.

In a specific embodiment, the probe used in a method of the invention is selected from Tables 1, 2, 3, 8, 9 and/or 12 wherein:

the probes specifically hybridizing to the K103N/R target sequences are selected from the following list: c103w62, c103w62b, c103w116, c103w49, c103w115, c103w55, c103w104, c103w52, c103w107, c103w107b, c103w92, c103w97, c103m26, c103m14, c103m22, c103w66, c103w36, c103w36b, c103w65, c103w121;

the probes specifically hybridizing to the V106A/I/L target sequences are selected from the following list: c103w62, c103w62b, c103w116, c103w49, c103w115, c103w55, c103w104, c103w52, c103w107, c103w107b, c103w92, c103w97, c103m26, c103m14, c103m22, c103w66, c103w36, c103w36, c103w65, c103w121;

the probes specifically hybridizing to the Y181C/I target sequences are selected from the following list: c181w3, c181w3b, c181w29, c181w29b, c181w33, c181w38, c181w39, c181w44, c181w53, c181w57, c181w65, c181w65b, c181w65c, c181w69, c181w133, c181w133b, c181w50, c181w97, c181w75, c181m7, c181m7b, c181m14, c181m22, c181m26, c181m144, c181m140;

the probes specifically hybridizing to the Q151 M/L target sequences are selected from the following list: c151w2, c151w51, c151w29, c151w29b, c151w31, c151w52, c151w53, c151m36, c151m48, c151m50;

the probes specifically hybridizing to the M184V/I target sequences are selected from the following list: c184w85, c184w85b, c184w86, c184w73bis, c184m42bis, c184m42bbis;

the probes specifically hybridizing to the Y188L target sequences are selected from the following list: c188ww1, c188ww12, c188ww24, c188ww29, c1 88ww3 1, c188ww40, c188ww45, c188ww58, c188wm63, c188wm72, c188wm73, c188wm70, c188mw76, 188mw76, c188mw83, c188mw86, c188mw86, c188mm77, 188mm77, c188wm82, c188wm128, c188wm129;

the probes specifically hybridizing to the G190A/S/R target sequences are selected from the following list: c188ww1, c188ww12, c188ww24, c188ww29, c188ww31, c188ww40, c188ww45, c188ww58, c188wm63, c188wm72, c188wm70, c188wm73, c188mw76, 188mw76, c188mw83, c188mw86, 188mw86, c188mm77, 188mm77, c188wm82, c188wm128, 188wm129;

the probes specifically hybridizing to the T215Y/F/D/S/A target sequences are selected from the following list: c215w145, c215w11, c215m99, c215m139, c215m108, c215m136, c215m84, c215m82, c215m77, c215m121, c215m115, c215m90, c215m95, c215m106.

A particularly preferred embodiment of the present invention is a method for determining the susceptibility to antiviral drugs of an HIV isolate via the detection of mutations associated with anti-HIV resistance, using a set of probes as defined above, wherein said set of probes is characterized as being chosen such that for a given mutation disclosed in any of FIGS. 1, 2 and/or 3, or Tables 1, 2, 3, 8, 9 and/or 12 the following probes are included in said set:

at least one probe for detecting the presence of drug induced mutation at said position;

at least one probe for detecting the presence of a wild-type sequence at said position;

preferably also (an) additional probe(s) for detecting wild-type and mutant polymorphisms at positions surrounding the mutation position.

Inclusion of the latter two types of probes greatly contributes to increasing the sensitivity of the assay. Such a design is presented in Tables 5, 6 and 13. It has to be understood that combinations of probes of the invention, other than those presented in the designs exemplified in Tables 5, 6 and 13, are possible and fall within the scope of the present inventions.

“Polymorphisms”, in the present context, relate to nucleotide changes or variations in the nucleotide sequence that do not result in amino acid changes and concomitant resistance building. They are referred to as wild-type polymorphisms. “Polymorphisms”, in a more general context, can also occur in mutated codons associated with resistance to antiviral drugs. The latter are referred to as mutant polymorphisms.

The term “polymorphic nucleotide” indicates a nucleotide in the HIV RT gene of a particular HIV virus that is different from the nucleotide at the corresponding position in at least one other HIV virus.

The term “polymorphic nucleic acid” refers to a nucleic acid comprising at least one polymorphic nucleotide.

The term “nucleic acid” refers to a single-stranded or double-stranded nucleic acid sequence. Both oligonucleotides and polynucleotides are included in this term. A nucleic acid that is up to about 100 nucleotides in length is often referred to as an oligonucleotide. A nucleic acid may consist of deoxyribonucleotides or ribonucleotides, nucleotide analogues or modified nucleotides, or may have been adapted for therapeutic purposes. A nucleic acid may also comprise a double-stranded cDNA clone that can be used for cloning purposes, or for in vivo therapy, or prophylaxis.

In an embodiment of the invention, the mutations at codon 103, 106, 151, 181, 184, 188, 190 and/or 215 of the HIV RT gene are detected by hybridization with at least one probe, preferably at least 2, more preferably at least 3, 4, 5, 6, 7, 8, 9, 10, 11, 12, 13 14, 15, 16, 17, 18, 19, 20 or more oligonucleotide probes. In a preferred embodiment of the present invention, probes for the detection of codons 103 and 106 were optimized to span the region from codon 103 to 106, so that information on both codons is obtained from a single probe. In another preferred embodiment of the present invention, probes for the detection of codons 188 and 190 were optimized to span the region from codon 188 to 190, so that information on both codons is obtained from a single probe.

A preferred embodiment of the present invention relates to a method as indicated above, further characterized in that said probes are optimized for simultaneous hybridization to their target regions under the same hybridization and wash conditions (for instance in a LiPA format (see below) or via the HIV PRT GeneChip assay (GeneChip, Affymetrix, Santa Clara, Calif.) allowing the simultaneous detection of a number of polymorphic regions.

The present invention also relates to the olignonucleotides used as probes to perform any method as described above.

The present invention also relates to a composition comprising any of the probes as described above or defined in Tables 1 to 14.

The reverse hybridization approach implies that the probes are immobilized to certain locations on a solid support. Poly(dt) tails or the like can be provided, either chemically or enzymatically, to enhance binding to the solid support. In this approach, the target DNA is labeled (via the labeling of the primers) in order to enable the detection of the hybrids formed. Alternatively, the probes could be labeled. In another embodiment, hybridization is inferred by chemiluminescence, electrochemically, measuring impedance or by any other means known in the art. The presence/absence of a hybridization signal indicates the presence/absence of the polynucleotides envisaged and any mutations and/or polymorphisms therein.

The term “biological sample” as used in the present invention refers to any biological material (tissue or fluid) taken either directly form the infected human being, or after culturing (enrichment) and containing HIV nucleic acid sequences. Biological material may be e.g. expectoration's of any kind, broncheolavages, blood, skin tissue, biopsies, sperm, lymphocyte blood culture material, colonies, liquid cultures, fecal samples, urine, hepatocytes, etc. More particularly “biological sample” refers to blood, serum or plasma samples.

The HIV RNA can be released, concentrated and/or isolated from the biological sample by any method known in the art. Currently, various commercial kits are available such as the ‘QIAamp Viral RNA Mini Spin Protocol’ from Qiagen (Hilden, Germany) and the ‘High Pure Viral Nucleic Acid Kit’ (Boehringer Molecular Biochemicals, Brussels, Belgium) for the isolation of nucleic acids from blood samples. Other well-known procedures for isolation of RNA from a biological sample are available (Sambrook et al., 1989).

The HIV RT gene or any relevant part thereof, present in said biological sample, can be amplified by polymerase chain reaction (PCR; Saiki et al., 1988), linked linear amplification (Reyes et al, 2001), ligase chain reaction (LCR; Landgren et al., 1988; Wu & Wallace, 1989; Barany, 1991), nucleic acid sequence-based amplification (NASBA; Guatelli et al., 1990; Compton, 1991), transcription-based amplification system (TAS; Kwoh et al., 1989), strand displacement amplification (SDA; Duck, 1990) or amplification by means of Q13 replicase (Lomeli et al., 1989) or by any other suitable method known in the art, that allows the amplification of nucleic acid molecules. Also TMA (Guatelli et al., 1990) or bDNA (Sanchez-Pescador et al., 1988; Urdea et al., 1991) techniques can be used in the method of the present invention. Commercial kits like for instance the ‘QIAGEN OneStep RT-PCR Kit’ s (Qiagen, Hilden, Germany) and the ‘Titan One Tube RT-PCR System’ (Boehringer Molecular Biochemicals, Brussels, Belgium) are suited for the amplification of the HIV RT gene.

The “part” of the RT gene to be amplified refers to the regions in the RT gene harboring mutations associated with resistance to antiviral drugs as described above and is particularly comprised between codons 9 and 270 of the RT gene or between codons 22 and 234. The RT gene part amplified for the present invention comprises at least codons 103, 106, 151, 181, 184 and/or 215 and possibly includes codons 188 and/or 190 of the RT gene. The numbering of HIV-1 RT gene encoded amino acids is as generally accepted in literature.

The term “primer” refers to a single-stranded oligonucleotide sequence capable of acting as a point of initiation for synthesis of a primer extension product or amplification product that is complementary to the nucleic acid strand to be copied. The length and the sequence of the primer must be such that they allow priming of the synthesis of the extension products. Preferably, the length of the primer is about 5-50 nucleotides. More preferably, the length of the primer is about 10-30 nucleotides. Most preferably, the length of the primers is about 15-25 nucleotides. Specific length and sequence will depend on the complexity of the required DNA or RNA target, as well as on the conditions at which the primer is used, such as temperature and ionic strength, and on the nature of the probe used.

The expression “primer set” refers to a pair of primers allowing the amplification of the HIV RT gene or part thereof. A primer set always consists of a forward primer (sense primer or 5′ primer) and a reverse primer (antisense primer or 3′ primer). In a preferred embodiment, the present invention relates to a method as described above, characterized further in that at least one of the primers used in step (ii) is selected from table 4 (AZT 16-bio, AZT 21-bio, AZT 35-bio, AZT 4-bio; SEQ ID NO: 1 to SEQ ID NO: 4).

More particularly, the present invention relates to a method as described above characterized further in that the set of primers consists of the following 2 primers:

AZT 1 6-bio as forward primer and AZT 21 -bio as reverse primer; and/or

AZT 35-bio as forward primers and AZT 35-bio as reverse primer.

Other primers that are suited for the amplification of the gene part of interest, namely that part of the HIV RT gene that contains at least one of the codons of the invention that have been associated with HIV drug resistance, are considered to fall under the scope of the present invention.

Another embodiment of the present invention relates to said primers or primer sets and/or their use in the methods described above. The skilled man will understand that these primers (SEQ ID NO: 1 to 4) may be adapted by addition or deletion of one or more nucleotides at their extremities. Such adaptations may be required, for instance, if the conditions of amplification are changed, if the amplified material is RNA instead of DNA, as is the case, for example, in the NASBA system. The fact that amplification primers do not have to match exactly with the corresponding target sequence in the template to warrant proper amplification is amply documented in the literature (Kwok et al., 1990). However, when the primers are not completely complementary to their target sequence, it should be taken into account that the amplified fragments will have the sequence of the primers and not of the target sequence. The nucleic acids in the sample to be analyzed may be either DNA or RNA, e.g. genomic DNA, messenger RNA, viral RNA or amplified versions thereof. These molecules are also termed polynucleic acids.

The primers and/or probes of the invention may be labeled or unlabeled. The term “labeled” refers to the use of a nucleic acid modified in such way as to allow its discrimination from an analogous nucleic acid without said modification. There are many methods known in the art to introduce said labels. An example of labeling is via the incorporation of labeled nucleotides during the polymerase step of the amplification such as illustrated by Saiki et al. (1988) or Bej et al. (1990) resulting in labeled primers. The nature of the label may be isotopic (³²P, ³⁵S, etc.) or non-isotopic (biotin, digoxigenin, etc.).

The oligonucleotides used as primers or probes may also contain or consist of nucleotide analogues such as phosphorothiates (Matsukura et al., 1987), alkylphosphorothiates (Miller et al., 1979) or peptide nucleic acids (Nielsen et al., 1991; Nielsen et al., 1993) or may contain intercalating agents (Asseline et al., 1984). The introduction of these modifications may be advantageous in order to positively influence characteristics such as hybridization kinetics, reversibility of the hybrid-formation, biological stability of the oligonucleotide molecules, etc.

As most other variations or modifications introduced into the original DNA sequences of primers and probes, these variations will necessitate adaptations with respect to the conditions under which the oligonucleotide should be used to obtain the required specificity and sensitivity. The eventual results of the priming or hybridization with these modified oligonucleotides, however, should be essentially the same as those obtained with the unmodified oligonucleotides.

The present invention describes in detail genetic detection of mutations associated with anti-HIV resistance. The present invention describes analysis of the presence of wild-type, polymorphic and/or mutant nucleic acids and the presence of combined mutations and/or polymorphisms. The present inventors have shown that such a combined determination of different wild-type, mutant and/or polymorphic sequences allows a more specific and more sensitive determination of anti-HIV drug resistance. Said analysis can be done by any other method known in the art, such as duplex analysis of the PCR products (Clay et al., 1994), single-stranded conformational polymorphism analysis of the PCR product (PCR-SSCP; Yoshida et al., 1992), sequence-based typing (SBT; Santamaria et al., 1992 and 1993), the use of sequence specific primers in PCR reaction (PCR-SSP; Olerup and Zetterquist, 1991), the use of PCR in combination with sequence-specific oligonucleotide probing (PCR-SSOP; Saiki et al., 1986), TMA or bDNA techniques. Alternatively, the HIV-GenotypR method (GenotypR, Specialty Laboratories, Inc., Santa Monica, Calif.) or a GeneChip, analogous to the HIV PRT GeneChip assay (GeneChip, Affymetrix, Santa Clara, Calif.) could be used (Wilson, 2000). Sequencing can be performed via any known sequencing method such as the enzymatic dideoxy method of Sanger et al (1977) or the chemical method of Maxam and Gilbert (1977, 1980). Kits and/or tools for thermal-cycle sequencing, solid-phase sequencing and automated sequencing are commercially available. Recent sequencing techniques provide for simultaneous sequencing in the 5′ and 3′ directions. Some examples of automated sequencers are the MicroGene Clipper™ 2 Dye and the MicroGene Blaster™ from the OpenGene™ system (Visible Genetics Inc., Toronto, Ontario, Canada) and the ABI PRISM® system (Perkin Elmer Inc., PE Biosystems, PE Applied Biosystems, Foster City, Calif., USA). Assay methods that rely on the formation of a hybrid between the nucleic acids in the biological sample and the oligonucleotide probe include Southern blot, Northern blot or dot blot format (Saiki et al., 1989), the unlabelled amplified sample being bound to a membrane, the membrane being incorporated with at least one labeled probe under suitable hybridization and wash conditions, and the presence of bound probe being monitored. An alternative is a “reverse” format, in which the amplified sequence contains a label. In this format, the selected probes are immobilized to certain locations on a solid support and the amplified polynucleic acids are labeled in order to enable the detection of the hybrids formed. The term “solid support” can refer to any substrate to which an oligonucleotide probe can be coupled, provided that it retains its hybridization characteristics and provided that the background level of hybridization remains low. Usually the solid substrate will be a microtiter plate (e.g. in the DEIA technique), a membrane (e.g. nylon or nitrocellulose) or a microsphere (bead) or a chip. Prior to application to the membrane or fixation it may be convenient to modify the nucleic acid probe in order to facilitate fixation or improve the hybridization efficiency. Such modifications may encompass homopolymer tailing, coupling with different reactive groups such as aliphatic groups, NH₂ groups, SH groups, carboxylic groups, or coupling with biotin, haptens or proteins.

The selection of the preferred probes of the present invention is based on a reverse hybridization assay using immobilized oligonucleotide probes present at distinct locations on a solid support (see below). More particularly the selection of preferred probes of the present invention is based on the use of the Line Probe Assay (LiPA) principle which is a reverse hybridization assay using oligonucleotide probes immobilized as parallel lines on a solid support strip (Stuyver et al. 1993, 1997; Maertens et al, 1998; International Application WO 94/12670). This approach is particularly advantageous since it is fast and simple to perform. The reverse hybridization format and more particularly the LiPA approach has many practical advantages as compared to other DNA techniques or hybridization formats, especially when the use of a combination of probes is preferable or unavoidable to obtain the relevant information sought.

It is to be understood, however, that any other type of hybridization assay or format using any of the selected probes as described further in the invention, is also covered by the present invention.

In a preferred embodiment, the present invention relates to a method, as indicated above, further characterized in that said probes are capable of simultaneously hybridizing to their respective target regions under appropriate hybridization conditions and wash conditions allowing the detection of more than one wild type codon and/or mutated codon at the same time.

More specifically, the present invention relates to a method as described above characterized further in that at least two of the mutations K103N/R, V106A/I/L, Q151NML, Y181C/I, M184V/I, Y188L, G19OA/S/R and/or T215Y/F/D/S/A are detected. More specifically, the present invention also relates to a method as described above, characterized further in that at least three of the above mutations are detected. More specifically, the present invention also relates to a method as described above, characterized further in that at least four of the above mutations are detected. More specifically, the present invention also relates to a method as described above, characterized further in that at least five of the above mutations are detected. More specifically, the present invention also relates to a method as described above, characterized further in that all six mutations K103N/R, V106A/I/L, Q151M/L, Y181C/I, M184V/1, Y188L, G190A/S/R and T215Y/F/D/S/A are detected. “Simultaneous detection”, in the context of the present invention should be interpreted as mutation detection in one single experiment but not necessarily in one and the same step of such an experiment. A mutation in one of the above codons is an indication that the HIV strain has become or is resistant to one or more of the (commonly) used antiviral drugs, such as nRTIs and/or nnRTIs.

Consequently, the method of the present invention can be used to screen for mutations in codon 103, 106, 151, 181, 184, 188, 190 and/or 215 of the HIV RT, associated with resistance to antiviral drugs. The term “anti-HIV drug” or “antiviral drug” in the present context refers to any nRTI, any rmRTI or any other RT inhibitor that causes a reduction of the viral RNA in the patient. The most common nRTIs are AZT, ABC, ddl, ddC, 3TC or D4T. The most common nnRTIs are NVP, DLV, EFZ (Schinazi et al., 1994 and Mellors et al., 1995). Said list is a non-exhaustive list and is therefore not limiting to the present invention. All said drugs are referred to by the term “drug of interest”.

The method of the invention can also be used in combination with a method for the detection of one or more other mutations that possibly are associated with resistance to other anti-HIV drugs. Thus, also probes that allow the detection of other mutations associated with resistance to fusion and integrase inhibitors can be added in the method of the invention.

Methods for detecting nucleotide changes in RT genes of other viruses which have been found to harbour a pattern of drug-resistance mutation similar to the one observed for HIV based on the same principles as set out in the present invention should be understood as also being covered by the scope of the present invention.

The present invention relates also to a kit for detection in a biological sample of mutations in the HIV RT, which are associated with antiviral drug resistance, comprising the following components:

(i) when appropriate, a means for releasing, isolating and/or concentrating the polynucleic acids present in the sample;

(ii) when appropriate, at least one of the sets of primers as defined above; (iii) at least one of the probes as defined above, possibly fixed to a solid support;

(iv) a hybridization buffer, or components necessary for producing said buffer;

(v) a wash solution, or components necessary for producing said solution;

(vi) when appropriate, a means for detecting the hybrids resulting from the preceding hybridization;

(vii) when appropriate, a means for attaching said probe to a solid support.

The term “hybridization buffer” means a buffer enabling a hybridization reaction to occur between the probes and the polynucleic acids present in the sample, or the amplified products, under the appropriate stringency conditions.

The term “wash solution” means a solution enabling washing of the hybrids formed under the appropriate stringency conditions.

The invention further relates to a diagnostic kit for the genetic detection of at least one of the mutations K103N/R, V106A/I/L, Y181IC/I, M184V/I, Y188L, G190A/S/R, T215Y/F/D/S/A and/or Q151M/L in the reverse transcriptase of the HIV strains present in a biological sample of a patient, with said mutations being associated with antiviral drug resistance, comprising the following components:

(i) when appropriate, a means for releasing, isolating and/or concentrating the polynucleic acids present in said biological sample;

(ii) when appropriate, at least one suitable primer pair;

(iii) at least one probe of the invention, possibly fixed to a solid support;

(iv) a hybridization buffer, or components necessary for producing said buffer;

(v) a wash solution, or components necessary for producing said solution;

(vi) when appropriate, a means for detecting the hybrids resulting from the preceding hybridization;

(vii) when appropriate, a means for attaching said probe to a known location on a solid support.

A line probe assay (LiPA) was designed for the screening for mutations and/or polymorphisms at interesting amino acids in the HIV RT gene. The principle of the assay is based on reverse hybridization (see above) of an amplified polynucleic acid fragment such as a biotinylated PCR fragment of the HIV RT gene onto short oligonucleotides. The latter hybrid can then, via a biotine-streptavidine coupling, be detected with a non-radioactive colour developing system.

The present invention further relates to a reverse hybridization method wherein said oligonucleotide probes are immobilized, preferably on a membrane strip.

The invention further relates to a line probe assay for the genetic detection of the K103N/R, V106A/I/L, Y181C/I, M184V/1, Y188L, G190A/S/R, T215Y/F/D/S/A and/or Q151M/L mutation in the reverse transcriptase of the HIV strains present in a biological sample of a patient, with said mutations being associated with antiviral drug resistance, comprising the following components:

(i) when appropriate, a means for releasing, isolating and/or concentrating the polynucleic acids present in a biological sample of the patient;

(ii) when appropriate, at least one suitable primer pair;

(iii) at least one probe specifically hybridizing with the K103N/R target sequences shown in FIG. 1 and selected from: c103w62, c103w62b, c103w116, c103w49, c103w115, c103w55, c103w104, c103w52, c103w107, c103w107b, c103w92, c103w97, c103m26, c103m14, c103m22, c103w66, c103w36, c103w36b, c103w65, c103w121, fixed to a solid support; and/or

(iv) at least one probe specifically hybridizing with the V106A/I/L target sequences shown in FIG. 1 and selected from: c103w62, c103w62b, c103w116, c103w49, c103w115, c103w55, c103w104, c103w52, c103w107, c103w107b, c103w92, c103w97, c103m26, c103m14, c103m22, c103w66, c103w36, c103w36b, c103w65, c103w121, fixed to a solid support; and/or

(v) at least one probe specifically hybridizing with the Y181C/I target sequences shown in FIG. 1 and selected from: c181w3, c181w3b, c181w29, c181w29b, c181w33, c181w38, c181w39, c181w44, c181w53, c181w57, c181w65, c181w65b, c181w65c, c181w69, c181w133, c181w133b, c181w50, c181w97, c181w75, c181m7, c181m7b, c181m14, c181m22, c181m26, c181m144, c181m140, fixed to a solid support; and/or

(vi) at least one probe specifically hybridizing with the Q151M/L target sequences shown in FIG. 1 and selected from: c151w2, c151w51, c151w29, c151w29b, c151w31, c151w52, c151w53, c151m36, c151m48, c151m50, fixed to a solid support; and/or

(vii) at least one probe specifically hybridizing with the M184V/I target sequences shown in FIG. 2 and selected from: probes c184w85, c184w85b, c184w86, c184w73bis, c184m42bis, c184m42bbis, fixed to a solid support; and/or

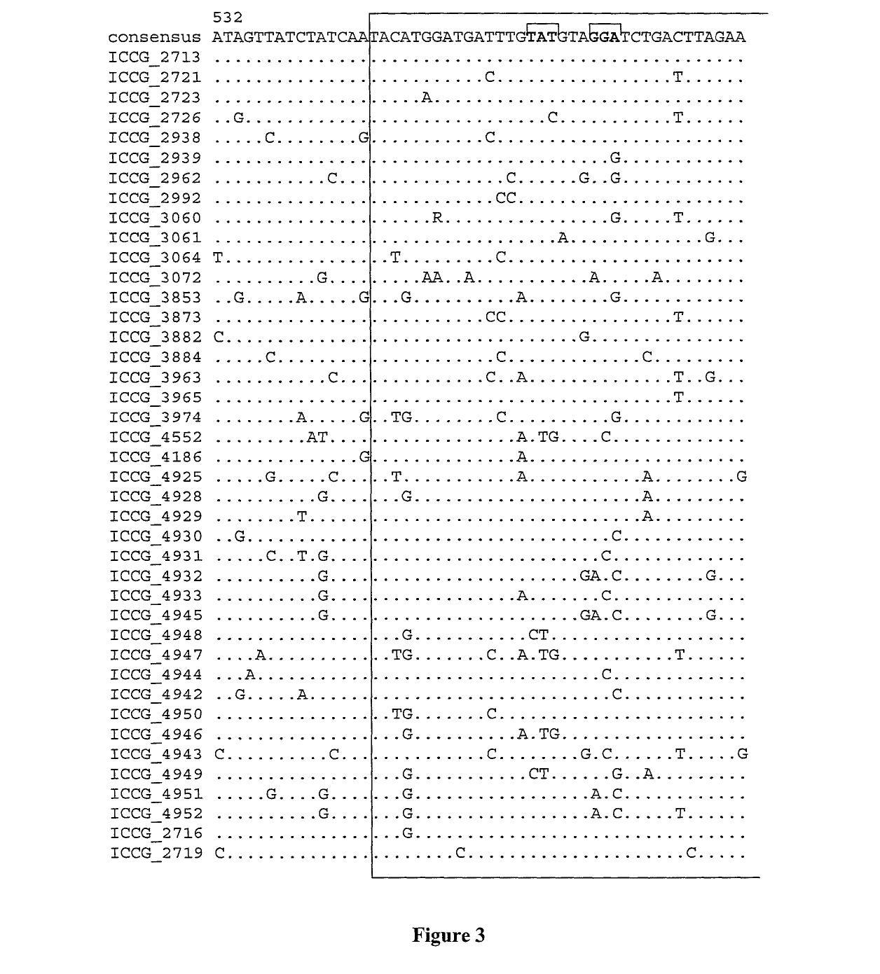

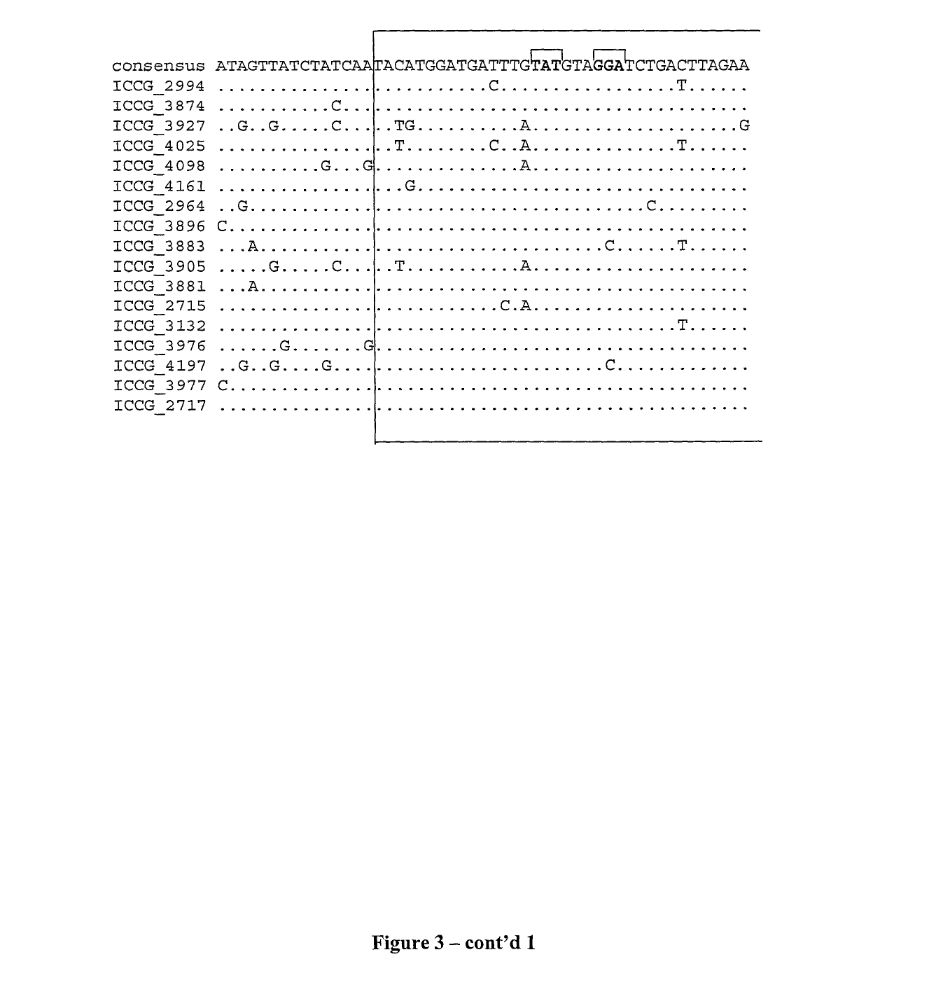

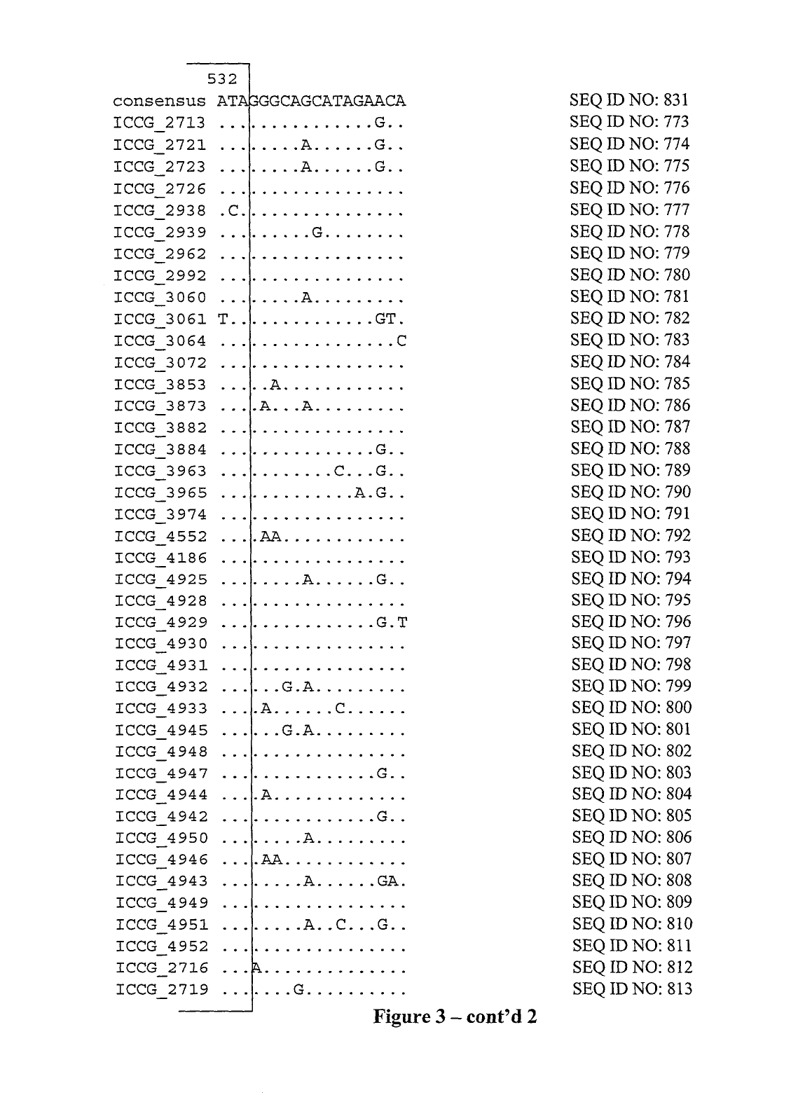

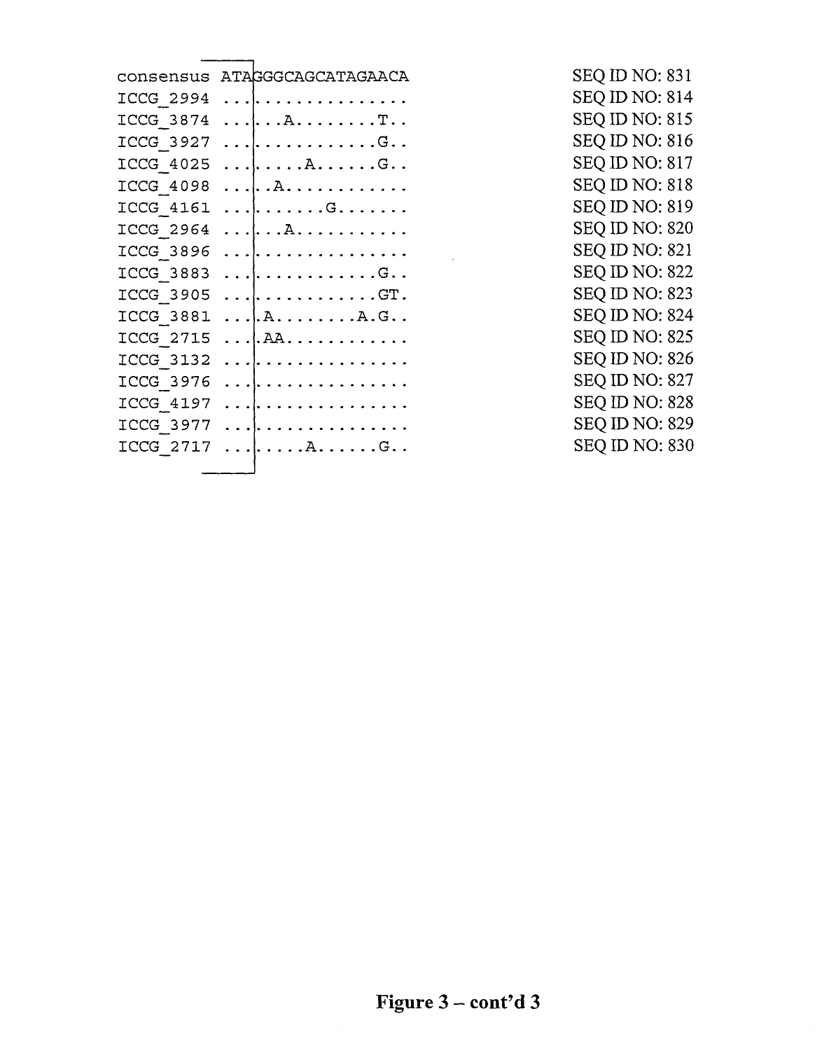

(viii) at least one probe specifically hybridizing to the Y188L target sequences shown in FIG. 3 and selected from: c188ww1, c188ww12, c188ww24, c188ww29, c188ww31, c188ww40, c188ww45, c188ww58, c188wm63, c188wm72, c188wm70, c188wm73, c188mw76, 188mw76, c188mw83, c188mw86, 188mw86, c188mm77, 188mm77, c188wm82, 188wm128, c188wm129, fixed to a solid support; and/or

(ix) at least one probe specifically hybridizing to the G190A/S/R target sequences shown in FIG. 3 and selected from: c188ww1, c188ww12, c188ww24, c188ww29, c188ww31, c188ww40, c188ww45, c188ww58, c188wm63, c188wm72, c188wm70, c188wm73, c188mw76, 188mw76, c188mw83, c188mw86, 188mw86, c188mm77, 188nmm77, c188wm82, c188wm128, c188wm129, fixed to a solid support; and/or

(x) at least one probe specifically hybridizing with the T215Y/F/D/S/A target sequences shown in FIG. 2 and selected from: c215w145, c215w111, c215m99, c215m139, c215m108, c215m136, c215m84, c215m82, c215m77, c215m121, c215m115, c215m90, c215m95, c215m106, fixed to a solid support;

(xi) a hybridization buffer, or components necessary for producing said buffer; (xii) a wash solution, or components necessary for producing said solution;

(xiii) when appropriate, a means for detecting the hybrids resulting from the preceding hybridization.

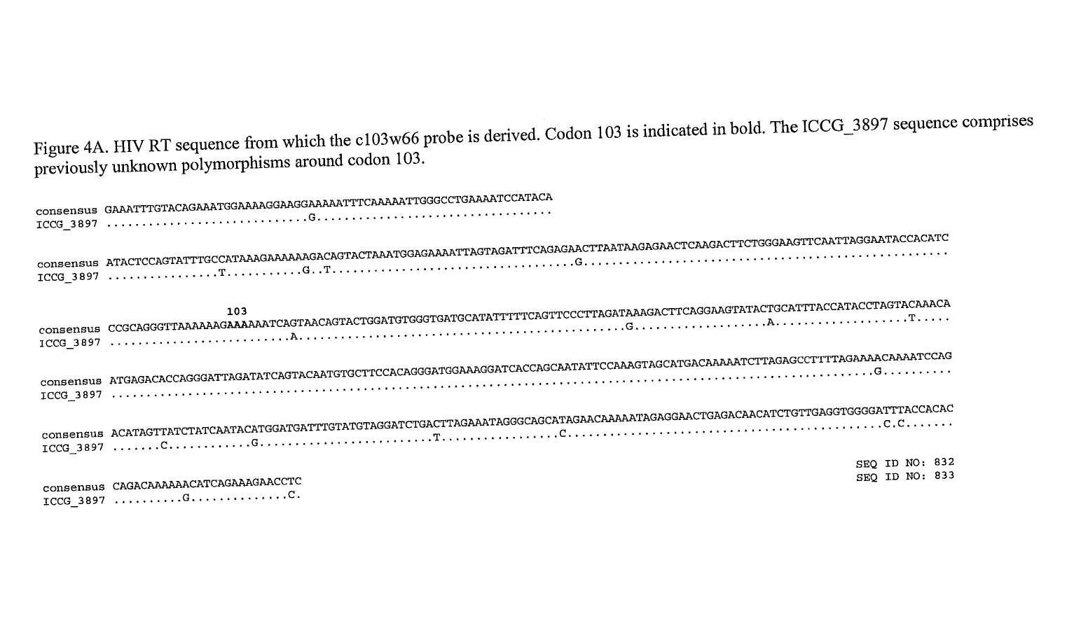

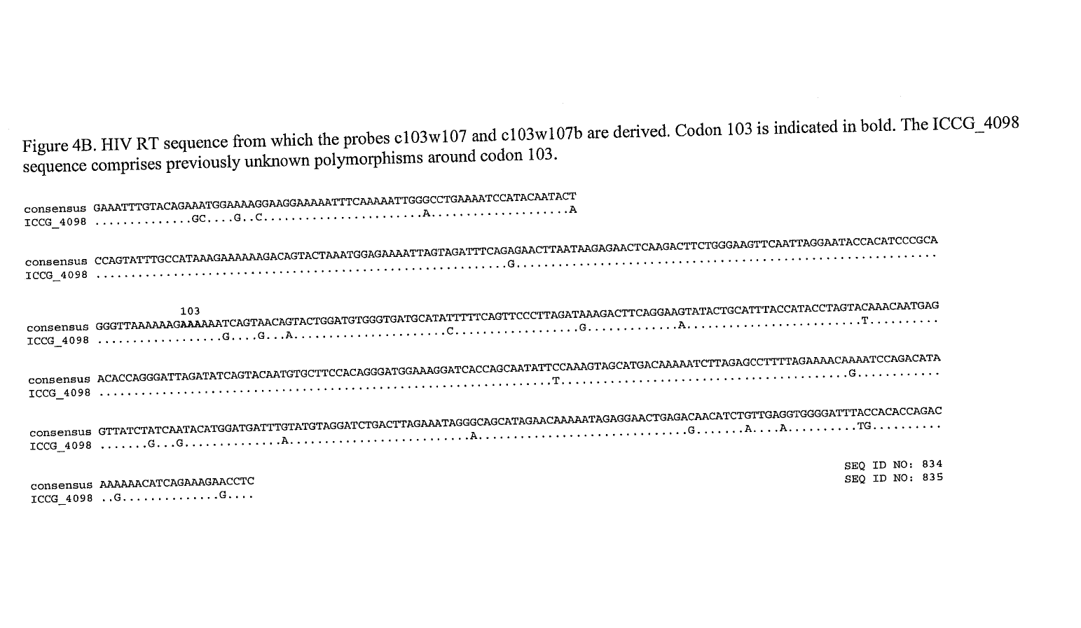

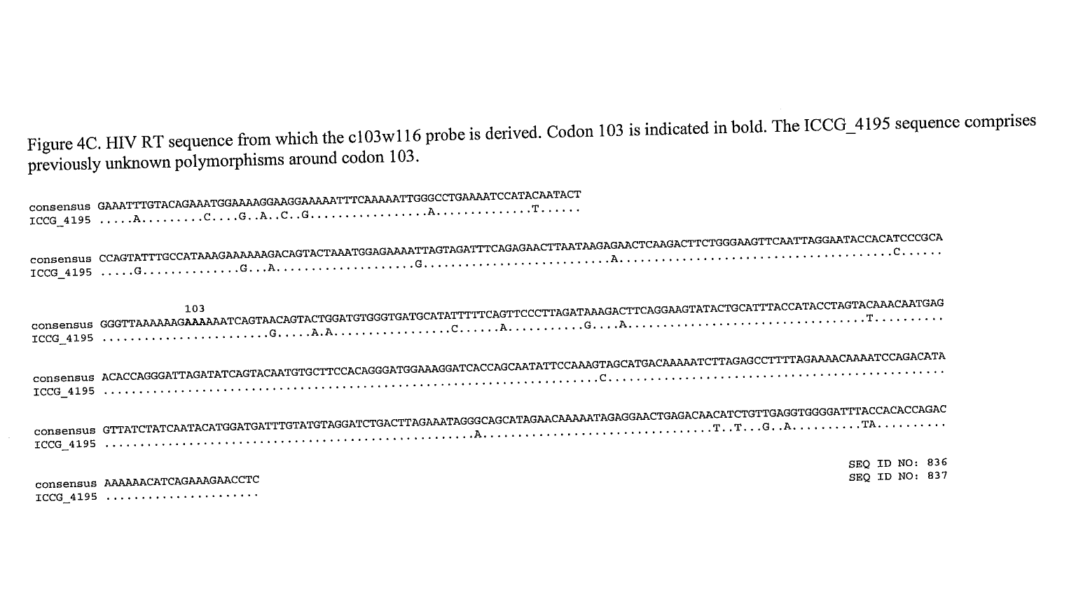

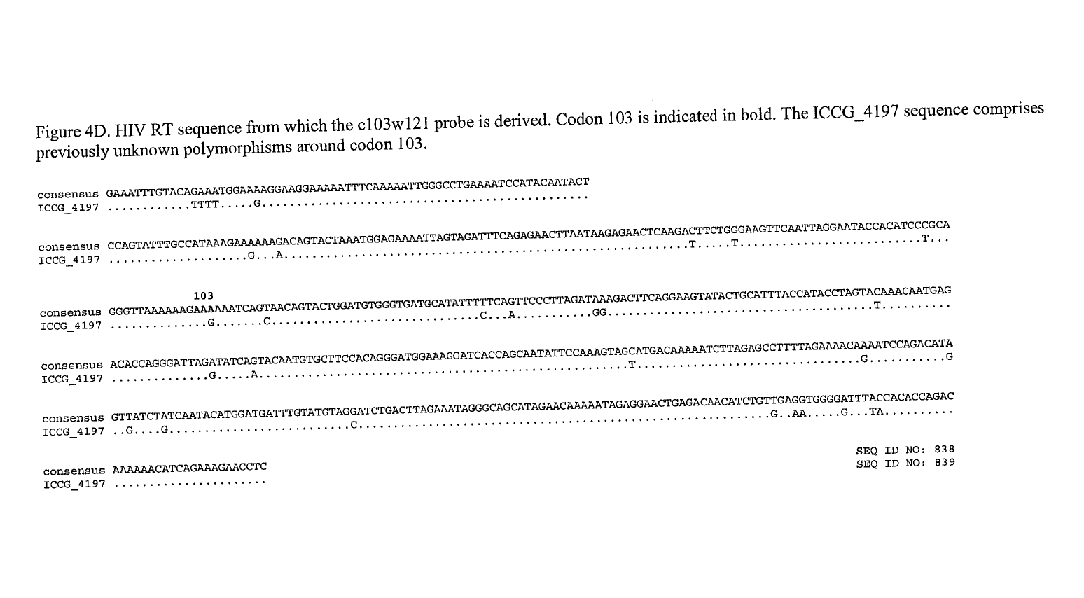

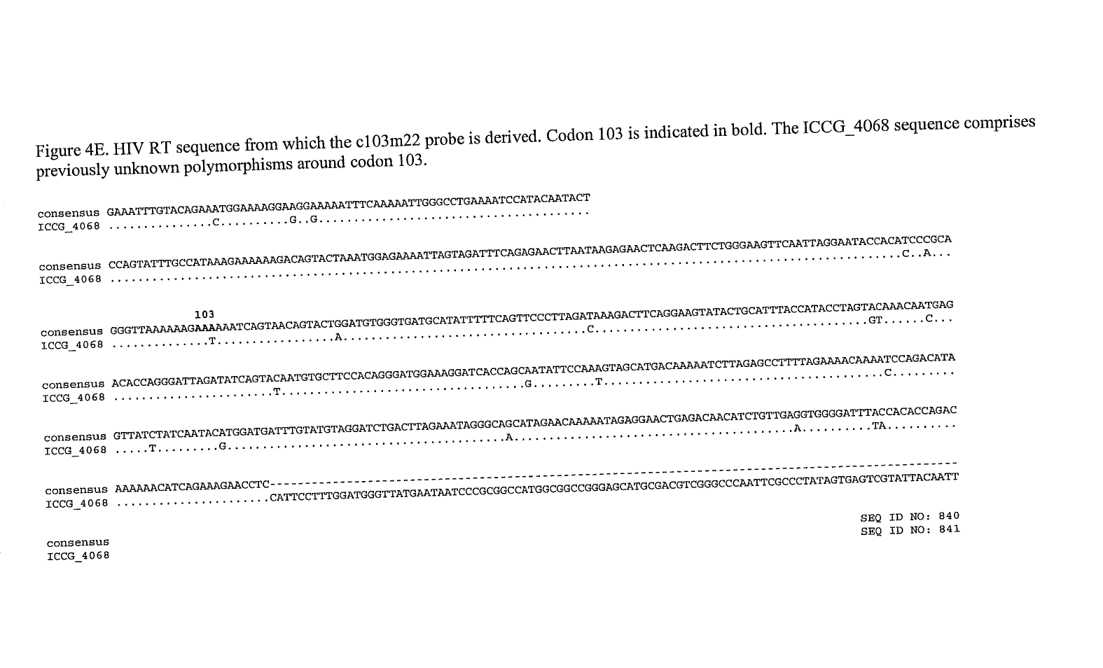

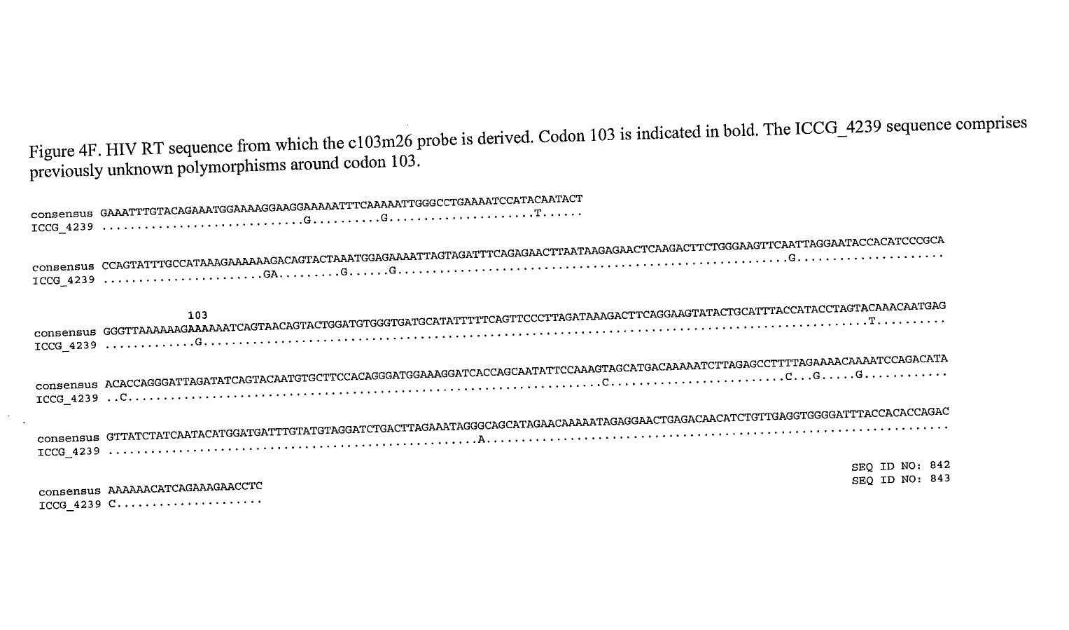

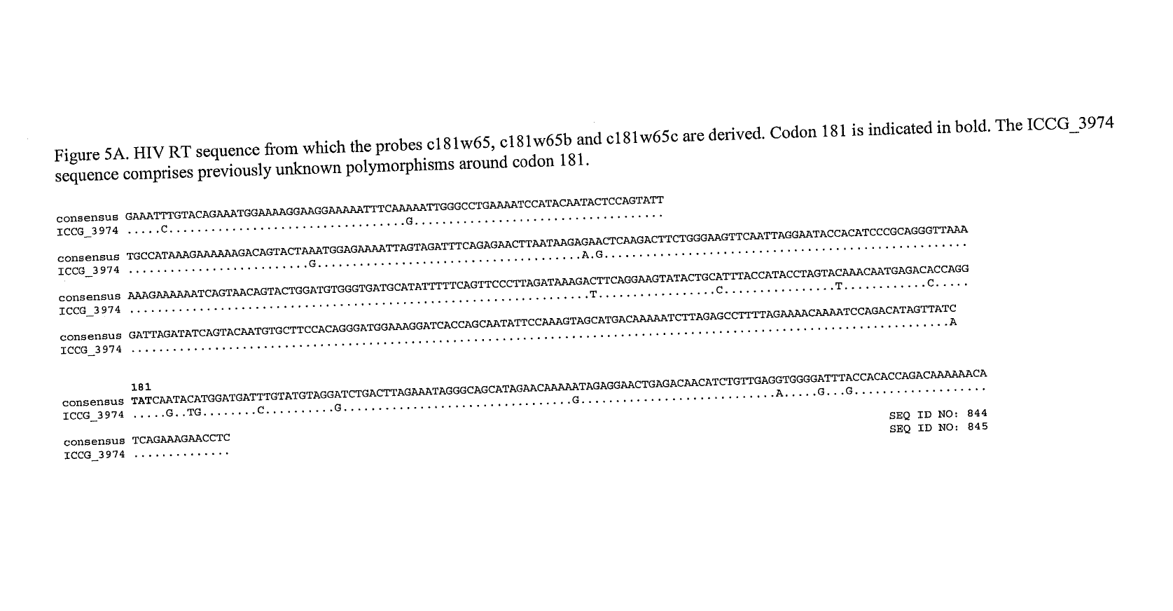

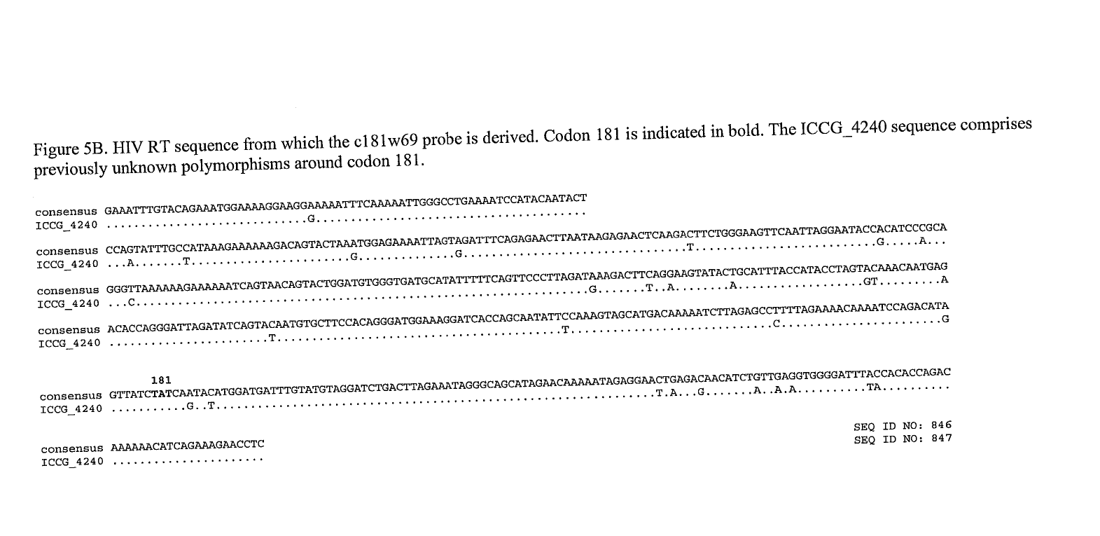

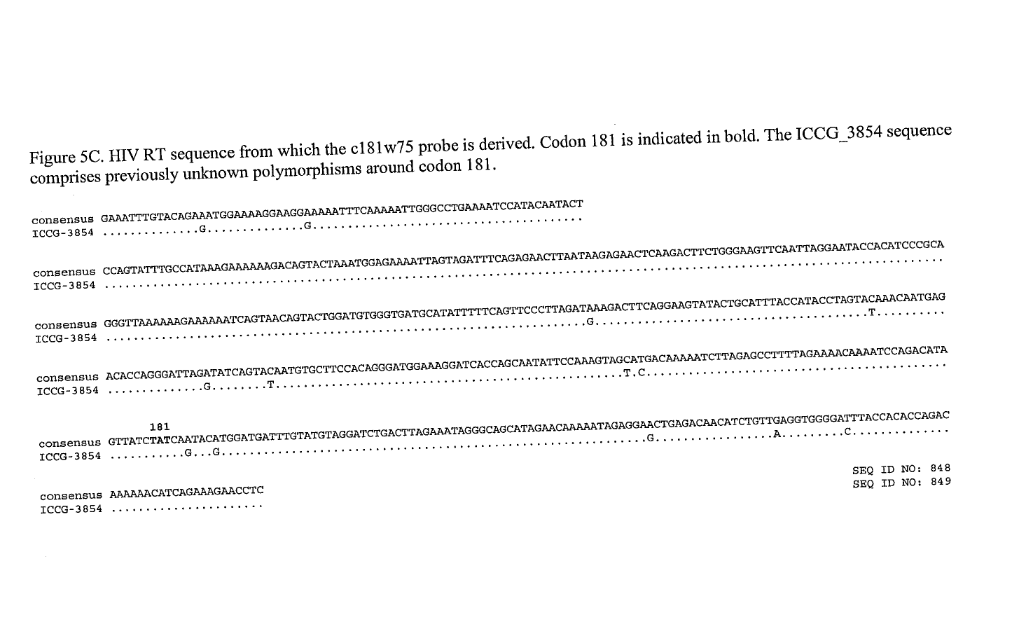

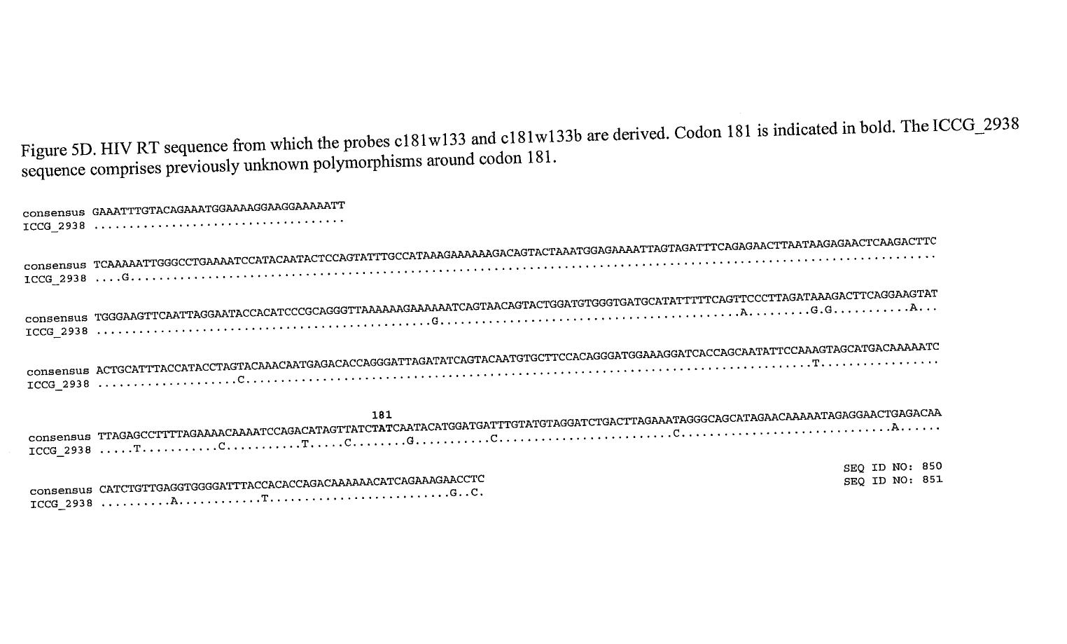

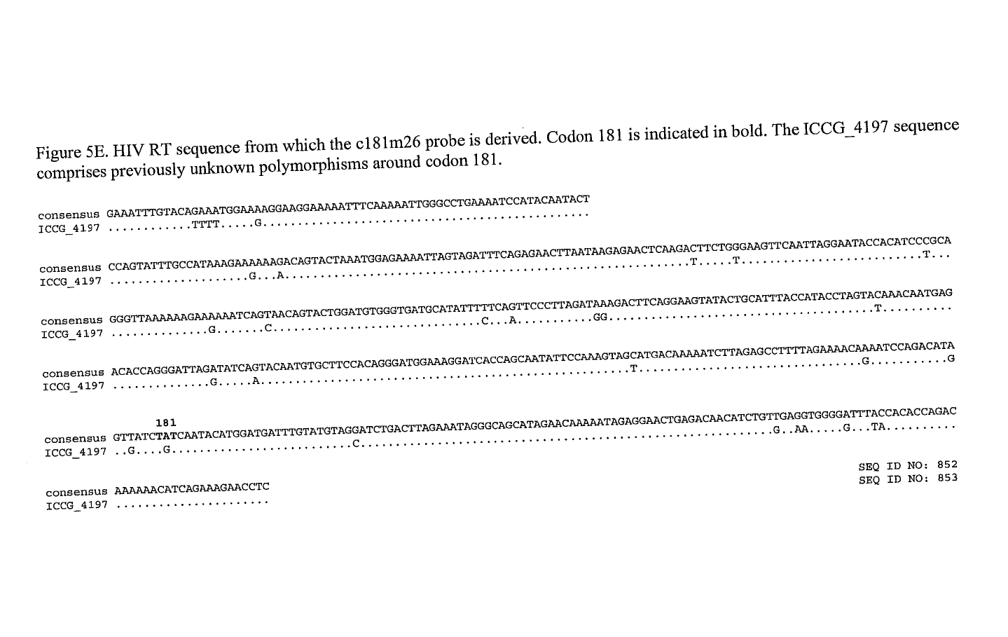

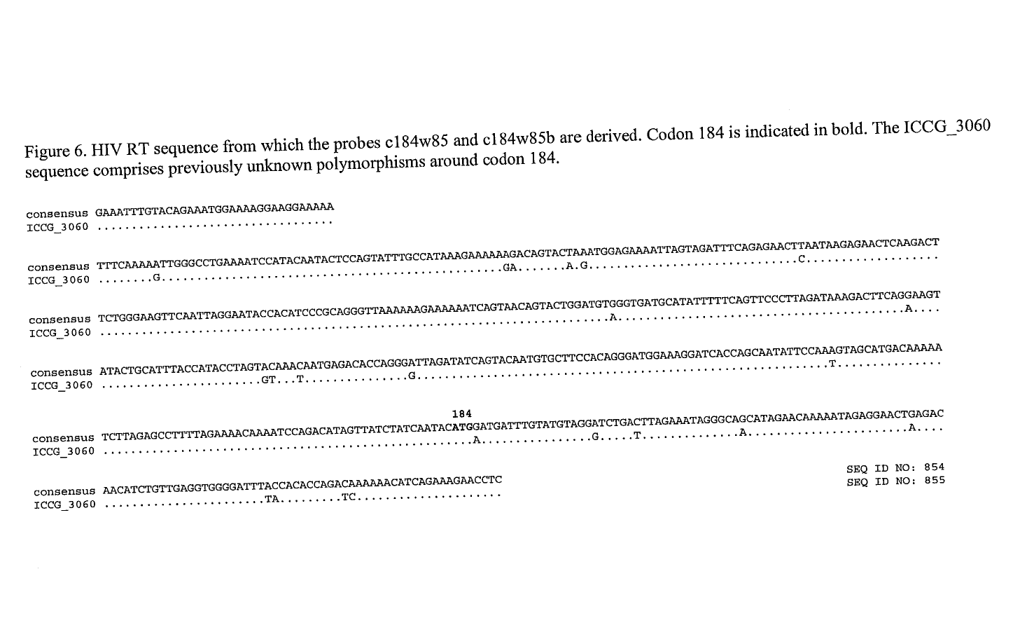

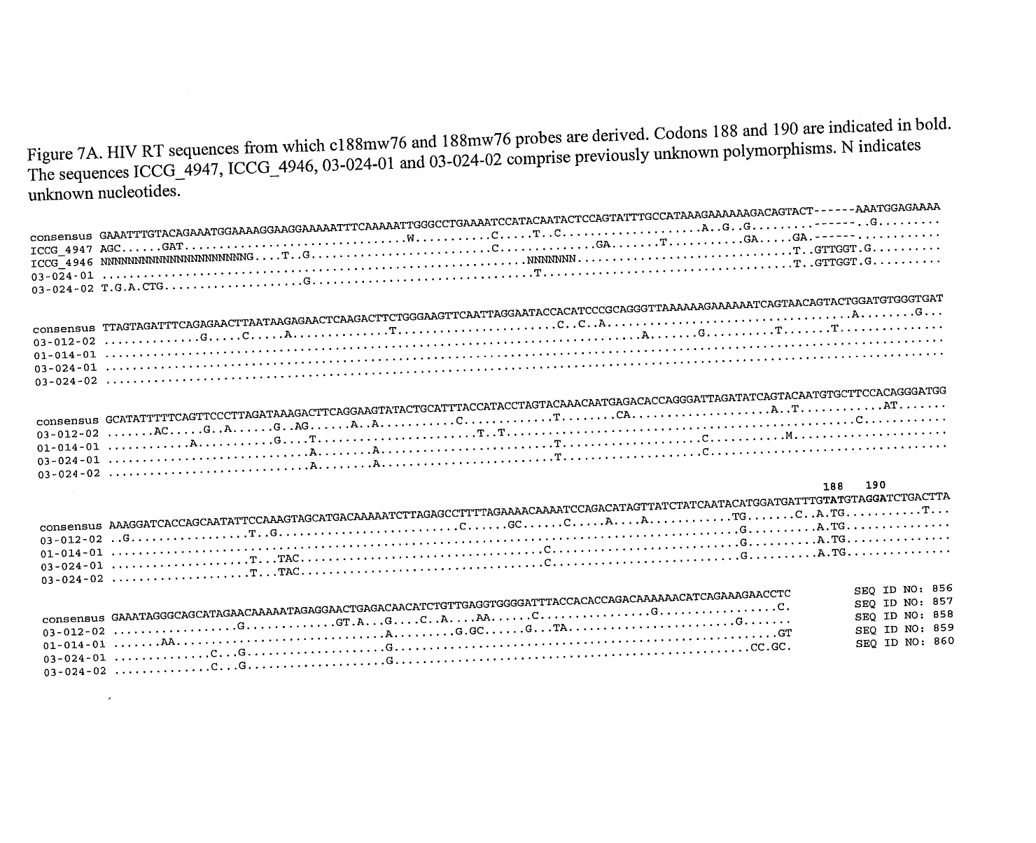

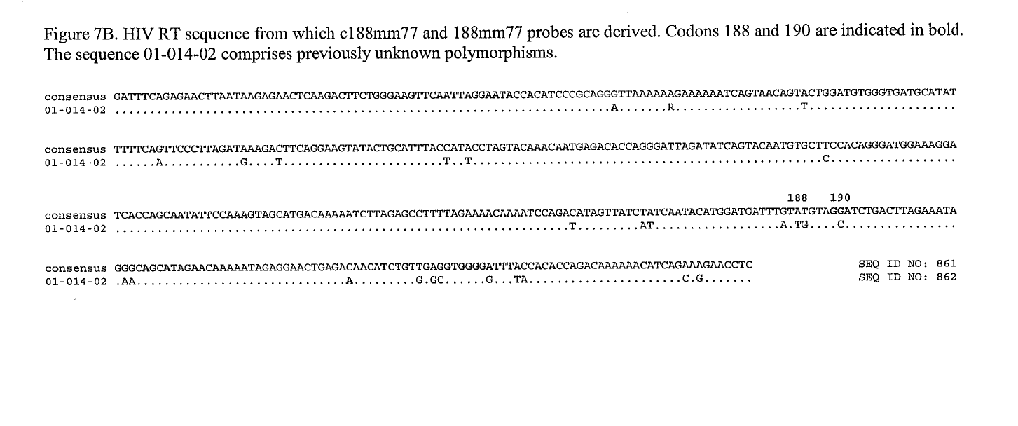

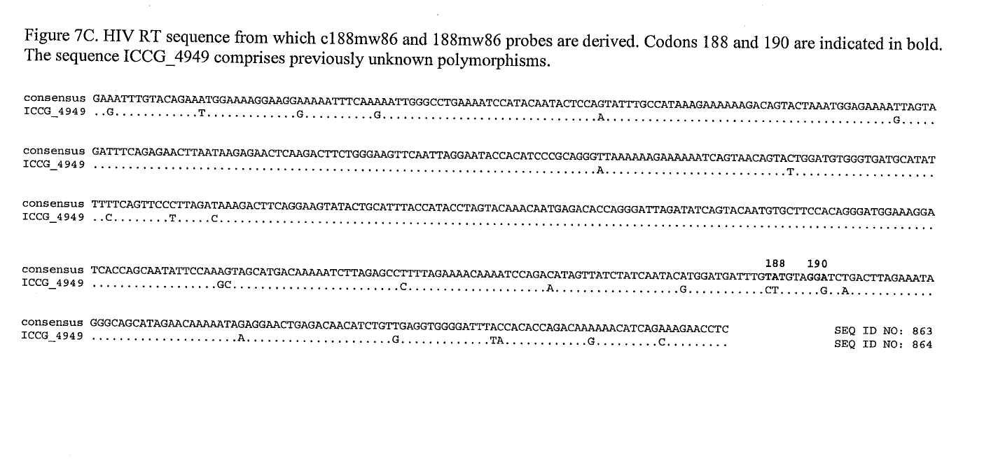

Some of the probes of the invention were designed based upon HIV RT gene sequences which comprise polymorphisms that were hitherto unknown (See FIGS. 4 to 7). The present invention also relates to said previously unknown HIV RT gene sequences. More particularly, the present invention relates to any nucleic acid comprising a nucleotide sequence selected from the group consisting of: 833, SEQ ID NO: 835, SEQ ID NO: 837, SEQ ID NO: 839, SEQ ID NO: 841, SEQ ID NO: 843, SEQ ID NO: 845, SEQ ID NO: 847, SEQ ID NO: 849, SEQ ID NO: 851, SEQ ID NO: 853, SEQ ID NO: 855, SEQ ID NO: 857, SEQ ID NO: 858, SEQ ID NO: 859, SEQ ID NO: 860, SEQ ID NO: 862, SEQ ID NO: 864, the complement thereof or a fragment thereof, wherein the fragment contains at least one polymorphic nucleotide.

Examples of probes derived from such previously unknown sequences are c103w66, c103w107b, c103w116, c103w121, c103m22, c103m26, c181w65c, c181w69, c181w75, c181w133, c181m26, c184w85b, c188mw76, 188mw76, c188mm77, 188mm77, c188mw86, 188mw86 (Table 14).

Accordingly, the present invention also relates to an oligonucleotide or a nucleic acid comprising a nucleotide sequence selected from c103w66, c103w107b, c103w116, c103w121, c103m22, c103m26, c181w65c, c181w69, c181w75, c181w133, c181m26, c184w85, c188mw76, 188mw76, c188mm77, 188mm77, c188mw86, 188mw86, the complement thereof or a fragment thereof, wherein the fragment contains at least one polymorphic nucleotide.

The invention further relates to the use of the above oligonucleotides and polynucleotides in a method for detection of mutations and/or polymorphisms in the HIV RT gene. Said method can be any method known in the art in which the presence of one of the above-mentioned nucleic acids is detected. In a preferred embodiment, the above nucleic acids are detected making use of a sequencing reaction or a hybridization reaction.

The word “comprise”, and variations such as “comprises” and “comprising”, will be understood to imply the inclusion of a stated integer or step or group of stated integers or steps but not to the exclusion of any other integer or step or group of integers or steps.

The disclosures of the various patent applications, patents and/or publications that are cited, as well as the references cited in these publications, are incorporated by reference herein. This, however, does not imply that the content of all of these disclosures is to be seen as part of common general knowledge.

The following examples only serve to illustrate the present invention. These examples are in no way intended to limit the scope of the present invention.

### EXAMPLES

### Example 1

### Selection of Suitable Probes for the Detection of Mutations at Codons 103/106, 151, 181, 184, 188/190 and 215 in the HIV RT Gene

a. Selection of Codons to Put on the Strip.

Mutations associated with anti-HIV resistance can be found at codons 98, 100, 101, 103, 106, 108, 179, 181, 188, 190 and 236. Published data from clinical studies (about 5000 sequences in the VircoGEN sequence collection and 127 virus sequences from 103 patients undergoing a nnRTI treatment in the Stanford database available on the web) and from a data set acquired from an in-house collection of samples were analyzed for mutations and/or polymorphisms at the above codon positions. In around 95% of clinical samples with mutations at these codons, mutations were found to occur at codons 103, 106, 181, 188 or 190. Codon 151, a multi-drug resistance codon, was included for its clinical relevance (see example 3). Further probes to codons 184 and 215 were considered necessary to optimize the detection method of WO 97/00211 in order to decrease the percentage indeterninates.

b. HIV RNA Purification and Amplification

Plasma samples were taken from HIV infected patients and stored at -20° C. until use. A collection of about 400 HIV sequences was used for the designing and optimization of probes. Said samples were obtained from Europe and the USA. Samples were from patients treated with AZT, ddl, ddC, D4T, 3TC, DLV, EFZ or several combinations of these prodrugs as well as from non-treated patients.

HIV RNA was isolated from the plasma samples by using the commercially available ‘High Pure Viral Nucleic Acid Kit’ (Boehringer Molecular Biochemicals, Brussels, Belgium) or the ‘QIAamp Viral RNA Mini Spin Protocol’ (Qiagen, Hilden, Germany) following the recommendations of the manufacturer.

The HIV reverse transcriptase (RT) gene was then amplified by using the commercially available ‘QIAGEN OneStep RT-PCR Kit’ (Qiagen, Hilden, Germany). 20 μl of template RNA was mixed with 10 μl 5× RT-PCR buffer, 2 μl 10 mM dNTP Mix (Qiagen, Hilden, Germany), 0.1 μl of 100U/μl HPRI (Amersham Pharmacia Biotech), 2 μl (50 pmol each) RT-PCR primers, 2 μl RT-PCR enzyme mix (Qiagen, Hilden, Germany) and 13.9 μl HPLC-grade H₂O (LABscan). RNAse free PCR tubes and a T3 Biometra Thermocycler were from Westburg (Leusden, The Netherlands).

PCR consisted of annealing at 57° C., extension at 72° C. and denaturation at 94° C., each step for 30 sec. First round PCR reactions contained 40 cycles, second round PCR 35 cycles. For the second round PCR, 2 μl of first round PCR product was mixed with 5 μl 10× Taq DNA polymerase buffer (Stratagene), 2 μl (50 pmol each) nested PCR primers, 0.4 μl 25 mM dNTP Mix (Pharmacia Biotech), 0.2 μl of 5U/μl Taq2000 Enzyme (Stratagene) and 40.4 μl HPLC-grade H₂O. The HIV RT region was amplified with the following primer combinations: outer sense primer AZT 16-bio: 5′-CCA GT(G/A) AAA TTA AAG CCA GGA ATG GAT GGC CC-3′ (SEQ ID NO: 1); outer anti-sense primer AZT 21-bio: 5′-ATC TGA CTT GCC CAA TT(T/C) AAT TT(T/C) CCC ACT AA-3′ (SEQ ID NO: 2); nested sense primer AZT 35-bio: 5′-AAA CAA TGG CCA TTG ACA GAA G-3′ (SEQ ID NO: 3); nested anti-sense primer AZT 4-bio: 5′-AGT TCA TAA CCC ATC CAA AG-3′ (SEQ ID NO: 4).

Nested amplification products of (primers included) 639 bp long, were analyzed on a 2% agarose gel, and visualized by ethidium bromide. In the case of LiPA experiments, primers were provided at their 5′ ends with a biotin group. Only clearly visible amplification products were used in the LiPA procedure. Quantification of viral RNA was obtained with the HIV Monitory test (Roche, Brussels, Belgium).

c. Plasmid Cloning and DNA Purification

Two μl of the amplification product was mixed with 1 μl pretreated EcoRV-cut pGemT vector (Promega, Leiden, The Netherlands) and ligated by means of the ‘Ready to Go’ T4 ligase (Pharmacia, Leusden, The Netherlands). After transformation in competent E. coli strains, single recombinant clones were selected, and plasmid DNA was purified with the ‘High Pure Plasmid Isolation Kit’ (Boehringer Molecular Biochemicals, Brussels, Belgium) or the ‘Qia prep 96 Turbo Bio Robot kit’ (Qiagen, Hilden, Germany). Inserts from recombinant clones were PCR-amplified by means of either plasmid-derived primers or the nested HIV primers.

d. Development of the Reference Panel and Probe Design

In the present context, the about 600 HIV sequences of the in-house collection that were analyzed, led to the development of a reference panel of 130 plasmids. All of these were sequenced. Double-stranded sequences were obtained from biotinylated PCR products or, in case of recombinant clones, by using vector-derived sequencing primers as described in Stuyver et al. (1996). The sequences with different motifs at codons 103, 106, 151, 181, 184, 188, 190 and 215 were retained. This led to a final selection of the 35, 43 and 58 HIV sequences, given in FIGS. 1, 2 and 3, on which basis the probes for the present invention were designed.

In principle, only probes that discriminate between one single nucleotide variation will be retained. However, for certain polymorphisms at the extreme ends of the probe, cross-reactivity can be tolerated. Specificity was reached for each probe individually after considering the % (G+C), the probe length, the final concentration of the buffer components, and hybridization temperature. These specific probes were evaluated by applying them to nitrocellulose membranes followed by reverse hybridization of the biotinylated PCR fragments generated from the plasma or serum samples (in a LiPA format), streptavidin-alkaline phosphatase incubation, and color development. Details on the probe optimization phase, LiPA strip production and reverse hybridization are described in Stuyver et al. (1996), Stuyver et al. (1997) and Van Geyt et al. (1998).

Probes were optimized to span the region from codon 103 to 106 and the region of codon 188 to 190, so that information on both codons is obtained from a single probe. In some cases two or more probes are used for detection in regions with multiple variation. Optimized probes were provided enzymatically with a poly(dt) tail using the TdT (Pharmacia) in a standard reaction condition, and purified via precipitation. Control lines for amplification and conjugate incubation (biotinylated DNA) were applied alongside.

In order to guarantee that the probes will be able to recognize the majority of HIV samples, a minimal amount of HIV sequences should initially be screened. A collection of at least 50, preferably at least 100 to 200, most preferably 300 to 400 or even more samples should be started with as was done for the present invention.

Below is explained how a highly-specific set of probes can be identified for heretofore unknown HIV sequences or how the set of probes can be expanded when upon analyses of the serum and plasma samples with this primary phase LiPA strip, it appears that some PCR products are not reactive with the primary phase selected probes. Non-reactive PCR products are then sequenced, revealing new motifs for which the corresponding probes are then designed. Including these newly designed probes on the LiPA strip will result in a decrease of non-reactivity of the samples. These steps of adding new motifs to the reference panel, and designing new probes are repeated as many times as needed.

### Example 2

### Validation of a LiPA Strip for Monitoring Drug-Resistance Due to Mutations at Codons 103, 106, 181, 188 and 190 of the RT Gene

a. Design of a LIPA for Monitoring Anti-HIV Drug-Resistance Due to Mutations in the RT Gene

By use of the sequences in the reference panel (see Example 1), specific probes for codon positions 103/106, 181, 188 and 190, covering both the wild-type and mutant motifs, were designed and validated. Probes were pooled according to their ability to detect the different wild type and mutant codons in the HIV reverse transcriptase, and applied on a strip. Several probes designed for different nucleotide polymorphisms but not introducing an amino acid change were pooled together and applied on one line. This finally resulted in a strip with 8 different probe lines with in total 37 specific probes for codon positions 103/106 and 181 (Table 5). On a separate strip, 6 different probe lines with in total 19 specific probes were applied for codon positions 188 to 190 (Table 13). A conjugation control and amplification control, lines 1 and 2, were included on the strip. Most of these probes covered specific minimal regions, which could be delineated as regions spanning at least positions 304 to 315, 303 to 316, 302 to 317, 307 to 318, 306 to 319, 305 to 320, 445 to 453, 444 to 454, 443 to 455, 449 to 455, 448 to 456, 447 to 457, 535 to 543, 534 to 544, 533 to 545, 538 to 546, 537 to 547, 536 to 548, 541 to 549, 540 to 550, 539 to 551, 541 to 566, 544 to 555, 543 to 556, 542 to 557, 550 to 561, 549 to 562, 548 to 563, 562 to 570, 561 to 571, 560 to 572, 637 to 645, 636 to 646, 635 to 647, 640 to 648, 639 to 649, 638 to 650, 643 to 651, 642 to 652, 641 to 653 and/or 634 to 656 of the HIV reverse transcriptase gene. The numbering of nucleotide positions as used in the present invention can be derived from FIGS. 1 and 2.

b. Validation of the Strip

In one study, a total of respectively 509 and 464 PCR products were evaluated for codon 103/106 and for codon 181. HIV-1 samples were obtained from Europe, the USA and Central America. The results of this study are summarized in Table 7.

A total of 262 of these clinical samples were analysed at all three codons 103, 106 and 181, using a prototype of the LiPA strip presented in Table 5. Hybridization was performed under conditions described in Stuyver et al (1997). The majority of samples were wild type at the three codons (157/262). Of the 34 samples that had a mutation, 3 had mutations at more than one of the codons investigated. The others had either only a mutation at codon 103 (14 samples), 106 (2 samples) or 181 (15 samples). 17% of the samples had an indeterminate result (45/262). 9/262 of the samples had an indeterminate result in both the 103-106 region and for codon 181. The others had an indeterminate only for the 103-106 region (10 samples) or for codon 181 (26 samples).

The INNO-LiPA assay could detect the presence of wild-type or mutated codons 103, 106 and 181 of HIV RT in more than 80% of the samples.

### Example 3

### Validation of a LiPA Strip for Monitoring Drug-Resistance Due to Mutations at Codon 151 of the HIV RT Gene

a. Design of a LiPA for Monitoring Anti-HIV Drug-Resistance Due to Mutations at Codon 151 in the RT Gene

Multi-Drug Resistant (MDR) HIV-1 isolates have been described. These MDR isolates are characterized by having mutations in their genome, compared to the wild type HIV-1 genome, which result in a set of amino acid changes. A key mutation leading to multi-drug resistance was found to be localized in codon 151 of the HIV-1 RT gene. Said mutation is reported to be rare, but has important outcome, as the virus has become resistant to not one but several nRTIs. As detecting these MDR isolates is clinically important, probes were designed that recognize wild-type and mutant HIV-1 isolates. Furthermnore, the presence of polymorphisms in the direct vicinity of codon 151 (codon 149) and at codon 151 have been described. 9 probes were finally withheld for the design of a LiPA strip for the detection of mutant or wild-type sequences at codon 151. The lay-out of this strip is given in Table 6. Probes were pooled according to their ability to detect the different wild type and mutant codons in the HIV reverse transcriptase, and applied on a strip. Several probes designed for different nucleotide polymorphisms but not introducing an amino acid change were pooled together and applied on one line. This finally resulted in a strip with 2 different probe lines with in total 9 specific probes. A conjugation control and amplification control, lines 1 and 2, were included on the strip. Most of these probes covered specific minimal regions, which could be delineated as regions spanning at least positions 449 to 455, 448 to 456, 447 to 457 of the HIV reverse transcriptase gene. The numbering of nucleotide positions as used in the present invention can be derived from FIG. 1.

b. Validation of the Strip

219 clinical samples were screened, using a prototype of the LiPa strip that is presented in Table 6, to analyze mutations at codon 151. Samples were obtained from Europe and the USA. Hybridization was performed under conditions described in Stuyver et al (1997). The vast majority of samples (96%) were wild-type at codon 151, which is in line with published data demonstrating that this mutation is very rare. 2% of the samples were mutant. 2% were indeterminate. No mixtures of wild type and mutant were detected. The LiPA strip was able to detect codon 151 in 98% of the cases.

c. LiPA Results in Comparison with Sequencing

For 102 of the 219 clinical samples studied, DNA sequences of the RT gene were also available, so that the LiPA assay could be compared with results from sequencing. For two samples with an indeterminate result, the sequence was determined afterwards. No discordant results between both assays were obtained. The LiPA assay of the present invention is thus a simple and accurate method to identify this MDR mutation at codon 151.

### Example 4

### Validation of a LIPA Strip for Monitoring Drug-Resistance Due to Mutations at Codons 184 and 215 of the HIV RT Gene

a. Design of a LIPA for Monitoring Anti-HIV Drug-Resistance Due to Mutations at Codons 184 and 215 in the RT Gene

Respectively 68 and 65 samples were selected from a larger collection of clinical samples obtained world-wide to further optimize detection at respectively codons 184 and 215 of the HIV RT gene. In the LiPA RT strip described in WO 97/00211, many indeterminate results were obtained at said codons. The strip described in WO 97/00211 is further referred to as the original LiPA RT strip. Probe optimization was done as described in Example 1. The 43 HIV-1 sequences that were retained for probe optimization are given in FIG. 2. Most of these probes covered specific minimal regions, which could be delineated as regions spanning at least positions 541 to 566 or 634 to 656 of the HIV reverse transcriptase gene. The numbering of nucleotide positions as used in the present invention can be derived from FIG. 2.

b. Validation of the Strip

Tables 10 and 11 show a comparison of the results obtained with the original LiPA RT strip and those obtained with the optimized strip, which includes further probes for codons 184 and 215. Dramatic improvements were obtained for codon 184, the key mutation for lamivudine (3TC) resistance. While initially 43 of the 68 samples had given a negative result, this number was decreased to only 4. For codon 215, the key mutation for resistance to zidovudine (AZT), the number of negative samples was reduced from 48 to 32 out of the 65 samples analysed.

### REFERENCES

Asseline U, Delarue M, Lancelot G, Toulme F, Thuong N (1984). Nucleic acid-binding molecules with high affinity and base sequence specificity : intercalating agents covalently linked to oligodeoxynucleotides. Proc. Natl. Acad. Sci. USA 81(11): 3297-301.

Barany F (1991). Genetic disease detection and DNA amplification using cloned thermostable ligase. Proc Natl Acad Sci USA 88: 189-193.

Bej A, Mahbubani M, Miller R, Di Cesare J, Haff L, Atlas R (1990). Multiplex PCR amplification and immobilized capture probes for detection of bacterial pathogens and indicators in water. Mol Cell Probes 4: 353-365.

Clay T M, Culpan D, Howell W M, Sage D A, Bradley B A and Bidwell J L (1994). UHG crossmatching. A comparison with PCR-SSO typing in the selection of HLA-DPB-compatible bone marrow donors. Transplantation 58: 200-207.

Compton J (1991). Nucleic acid sequence-based amplification. Nature 350: 91-92.

Duck P (1990). Probe amplifier system based on chimeric cycling oligonucleotides. Biotechniques 9: 142-147.

Guatelli J, Whitfield K, Kwoh D, Barringer K, Richman D, Gengeras T (1990). Isothermal, in vitro amplification of nucleic acids by a multienzyme reaction modeled after retroviral replication. Proc Natl Acad Sci USA 87: 1874-1878.

Hirsch M S, Brun-Vezinet F, D'Aquilla R T, Hammer S M, Johnson V A, Kuritzkes D R, Loveday C, Mellors J W, Clotet B, Conway B, Demeter L M, Vella S, Jacobsen D M and Richman D R (2000). Antiretroviral drug resistance testing in adult HIV-1 infection. Journal of the American Medical Association: 283(18): 2417-2426.

Iversen A. K. N., R. W. Shafer, K. Wehrly, M. A. Winters, J. I. Mullins, B. Chesebro, and T. Merigan (1996). Multidrug-resistant human immunodeficiency virus type 1 strains resulting from combination antiretroviral therapy. J. Virol. 70:1086-1090.

Kemp S, Bloor S, Van Cauwenberge A, De Koning I, Van den Eynde C, Larder B and Hertogs K (1999). Analysis of 5000 HIV-1 clinical samples reveals complex non-nucleoside RT inhibitor resistance patterns. Antiviral therapy 4 (suppl 1): 20.

Kwoh D, Davis G, Whitfield K, Chappelle H, Dimichele L, Gingeras T (1989). Transcription-based amplification system and detection of amplified human immunodeficiency virus type 1 with a bead-based sandwich hybridization fornat. Proc Natl Acad Sci USA 86: 1173-1177.

Kwok S, Kellogg D, McKinney N, Spasic D, Goda L, Levenson C, Sinisky J (1990). Effects of primer-template mismatches on the polymerase chain reaction: Human immunodeficiency views type 1 model studies. Nucl. Acids Res. 18: 999.

Landgren U, Kaiser R, Sanders J, Hood L (1988). A ligase-mediated gene detection technique. Science 241:1077-1080.

Lomeli H, Tyagi S, Printchard C, Lisardi P, Kramer F (1989). Quantitative assays based on the use of replicatable hybridization probes. Clin Chem 35: 1826-1831.

Maertens G and Stuyver L (1998). HCV genotyping by the line probe assay INNO-LiPA HCV II. In: Lau JYN, ed. Methods in Molecular Medicine. Vol 18: Hepatitis C Protocols. Humana Press, Totowa, NJ: pp. 183-198.

Matsukura M, Shinozuka K, Zon G, Mitsuya H, Reitz M, Cohen J, Broder S (1987). Phosphorothioate analogues of oligodeoxynucleotides: inhibitors of replication and cytopathic effects of human immunodeficiency virus. Proc. Natl. Acad. Sci. USA 84(21):7706-10.

Mellors J, Larder B, Schinazzi R (1995). Mutations in HIV-1 reverse transcripatse and protease associated with drug resistance. International Antiviral News 3: 8-13.

Miller P, Yano J, Yano E, Carroll C, Jayaram K, Ts'o P (1979). Nonionic nucleic acid analogues. Synthesis and characterization of dideoxyribonucleoside methylphosphonates. Biochemistry 18(23): 5134-43.

Nielsen P, Egholm M, Berg R, Buchardt O (1991). Sequence-selective recognition of DNA by strand displacement with a thymine-substituted polyamide. Science 254(5037):1497-500.

Nielsen P, Egholm M, Berg R, Buchardt O (1993). Sequence specific inhibition of DNA restriction enzyme cleavage by PNA. Nucleic Acids Res. 21(2):197-200.

Olerup O and Zetterquist H (1991). HLA-DRB1*01 subtyping by allele-specific PCR amplification: a sensitive, specific and rapid technique. Tissue Antigens 37: 197-204.

Reyes A A, Ugozzoli L A, Lowery J D, Breneman J W, Hixson C S, Press R D and Wallace R B (2001). Linked Linear Amplification: a new method for the amplification of DNA. Clinical Chemistry 47: 31-40.

Saiki R, Walsh P, Levenson C, Erlich H (1989). Genetic analysis of amplified DNA with immobilized sequence-specific oligonucleotide probes Proc Natl Acad Sci USA 86:6230-6234.

Saiki R K, Gelfland D H, Stoffel S, Scharf S J, Higuchi R, Horn G T, Mullis K B, and Erlich H A (1988). Primer-directed enzymatic amplification of DNA with a thermostable DNA polymerase. Science 239:487-491.

Sanchez-Pescador R, Stempien M S, Urdea M S (1988). Rapid chemiluminescent nucleic acid assays for detection of TEM-1 beta-lactamase-mediated penicillin resistance in Neisseria gonorrhoeae and other bacteria. J Clin Microbiol 26: 1934-1938.

Sambrook J, Fritsch E and Maniatis T (1989). Molecular cloning: a laboratory manual. Cold Spring Harbor Laboratory Press, Cold Spring Harbor, N.Y., USA.

Santamaria P, Boyce-Jacino MT, Lindstrom A L, Barbosa J J, Faras A J and Rich S S (1992). HLA class II “typing”: direct sequencing of DRB, DQB and DQA genes. Hum Immunol 33: 69-81.

Santamaria P, Lindstrom A L, Boyce J M, Jacino M T, Myster S H, Barbosa J J, Faras A J and Rich S S (1993). HLA class I sequence-based typing. Hum Immunol 37: 39-50.

Schinazi R, Larder B, Mellors J (1994). Mutations in HIV-1 reverse transcriptase and protease associated with drug resistance. International Antiviral News, 2:72-74.

Stuyver L, Rossau R, Wyseur A, et al (1993). Typing of hepatitis C virus isolates and characterization of new subtypes using a line probe assay. J. Gen. Virol. 74: 1093-1102.

Stuyver L, Wyseur A, van Arnhem W, Hernandez F and Maertens G (1996). A second generation line probe assay for hepatitis C virus. J Clin Microbiol 34: 2259-2266.

Stuyver L, Wyseur A, Rombout A, Louwagie J, Scarcez T, Verhofstede C, Rimland D, Schinazi RF, and Rossau R (1997). Line probe assay (LiPA) for the rapid detection of drug-selected mutations in the HIV-1 reverse transcriptase gene. Antimicrob Agents Chemother 41: 284-291.

Urdea M S, Horn T, Fultz T J, Anderson M, Running J A, Hamren S, Ahle D, Chang C A (1991). Branched DNA amplification multimers for the sensitive, direct detection of human hepatitis viruses. Nucleic Acids Symp Ser 24:197-200.

Van Geyt C, De Gendt S, Rombaut A, Wyseur A, Maertens G, Rossau R and Stuyver L (1998) A line probe assay for hepatitis B virus genotypes. In: R. F. Schinazi, J. P. Sommadossi, and H. Thomas (eds.). Therapies of viral hepatitis. International Medical Press, London, UK, pp. 139-145.

Van Geyt C, De Gendt S, Rombaut A, Wyseur A, Maertens G, Rossau R and Stuyver L (1998). A line probe assay for hepatitis B virus genotypes. In: Therapies of viral hepatitis. pp. 139-145. Eds. R F Schinazi, J P Sommadossi and H Thomas. International Medical Press, London, UK.

Wilson J W, Bean P, Robins T, Graziano F and Persing D H (2000). Comparative Evaluation of Three Human Immunodeficiency Virus Genotyping Systems: the HIV-GenotypeR method, the HIV PRT GenChip Assay, and the HIV-1 RT Line Probe Assay. Journal of Clinical Microbiology 38: 3022-3028.

Wu D, Wallace B (1989). The ligation amplification reaction (LAR)—amplification of specific DNA sequences using sequential rounds of template-dependent ligation. Genomics 4: 560-569.

Yoshida M, Kimura A, Numano F and Sasazuki T (1992). Polymerase-chain-reaction-based analysis of polymorphism in the HLA-B gene. Hum Immunol 34: 257-266.

1. Method for the detection of mutations associated with anti-HIV drug resistance in a patient by detection of at least one of the mutations K103N/R, V106A/I/L, Y181C/I, M184V/I, Y188L, G190A/S/R, T215Y/F/D/S/A and/or Q151M/L in the reverse transcriptase (RT) of HIV strains present in a biological sample of said patient, comprising the following steps:
(i) if needed, release, isolation and/or concentration of the polynucleic acids present in said biological sample;
(ii) if needed, amplification of the HIV reverse transcriptase gene or a part thereof in said biological sample with at least one suitable primer pair;
(iii) hybridization of the polynucleic acids in the sample, possibly released, isolated, concentrated and/or amplified via steps (i) and/or (ii), with at least one probe capable of specifically hybridizing with a target sequence in the HIV reverse transcriptase gene or the complementary or specifically hybridizing with a sequence wherein T in said target sequence is replaced by U, said target sequence being selected from the target sequences shown in FIGS. 1, 2 and/or 3;
(iv) detection of the hybrids formed in step (iii);
(v) inference, from the hybridization signal obtained in step (iv), of the presence or absence of the K103N/R, V106A/I/L, Y181C/I, M184V/I, Y188L, G190A/S/R, T215Y/F/D/S/A and/or Q151 MI/L mutation in the HIV reverse transcriptase and of possible anti-HIV drug resistance of the HIV strains present in said biological sample.

2. Method according to claim 1 further characterized in that the probe used in step (iii) is selected from tables 1, 2, 8 and/or 9, wherein:
the probes specifically hybridizing with the K103N/R target sequences are selected from the following: SEQ ID NO: 5 to SEQ ID NO: 106 and SEQ ID NO: 865 to SEQ ID NO: 867;
the probes specifically hybridizing with the V106A/I/L target sequences are selected from the following: SEQ ID NO: 5 to SEQ ID NO: 101;
the probes specifically hybridizing to the Y181C/I target sequences are selected from the following list: SEQ ID NO: 107 to SEQ ID NO: 240, SEQ ID NO: 868 to SEQ ID NO: 872 and SEQ ID NO: 883;
the probes specifically hybridizing to the Q151M/L target sequences are selected from the following list: SEQ ID NO: 241 to SEQ ID NO: 293 and SEQ ID NO: 873;
the probes specifically hybridizing to the M184V/I target sequences are selected from the following list: SEQ ID NO: 329 to SEQ ID NO: 424, SEQ ID NO2: 874 to SEQ ID NO: 878 and SEQ ID NO: 884;
the probes specifically hybridizing with the Y188L target sequences are selected from the following: SEQ ID NO: 618 to SEQ ID NO: 772;
the probes specifically hybridizing with the G190A/S/R target sequences are selected from the following: SEQ ID NO: 618 to SEQ ID NO: 772;
the probes specifically hybridizing with the T215Y/F/D/S/A target sequences are selected from the following: SEQ ID NO: 425 to SEQ ID NO: 572 and SEQ ID NO2: 879 to SEQ ID NO: 882.

3. Method according to any of claims 1 to 2 characterized further in that the probes used in step (iii) are at least one of the following combination of probes:
for the detection of the K103N/R mutation, probes: c103w62, c103w62b, c103w116, c103w49, c103w115, c103w55, c103w104, c103w52, c103w107, c103w107b, c103w92, c103w97, c103m26, c103m14, c103m22, c103w66, c103w36, c103w36b, c103w65, c103w121;
for the detection of the V106A/I/L mutation, probes: c103w62, c103w62b, c103w116, c103w49, c103w115, c103w55, c103w104, c103w52, c103w107, c103w107b, c103w92, c103w97, c1O3m26, c103m14, c103m22, c103w66, c103w36, c103w107b, c103w65, c103w121;
for the detection of the Y181C/I mutation, probes: c181w3, c181w3b, c181w29, c181w29b, c181w33, c181w38, c181w39, c181w44, c181w53, c181w57, c181w65, c181w65b, c181w65c, c181w69, c181w133, c181w133b, c181w50, c181w97, c181w75, c181m7, c181m7b, c181m14, c181m22, c181m26, c181m144, c181m140;
for the detection of the Q151M/L mutation, probes: c151w2, c151w51, c151w29, c151w29b, c151w31, c151w52, c151w53, c151m36, c151m48, c151m50; for the detection of the M184V/I mutation, probes: c184w85, c184w85b, c184w86, c184w73bis, c184m42bis, cl 84m42bbis;
for the detection of the Y188L mutation, probes: c188ww1, c188ww12, c188ww24, c188ww29, c188ww31, c188ww40, c188ww4S, c188ww58, c188wm63, c188wm70, c188wm72, c188wm73, c188mw76, 188mw76, c188mw83, c188mw86, 188mw86, c188mm77, 188mm77, c188wm82, c188wm128, c188wm129;
for the detection of the G190A/S/R mutation, probes: c188ww1, c188ww12, c188ww24, c188ww29, c188ww31, c188ww40, c188ww45, c188ww58, c188wm63, c188wm70, c188wm72, c188wm73, c188mw76, 188mw76, c188mw83, c188mw86, 188mw86, c188mm77, 188mm77, c188wm82, c188wm128, c188wm129;
for the detection of the T215Y/F/D/S/A mutation, probes: c215w145, c215w111, c215m99, c215m139, c215m108, c215m136, c215m84, c215m82, c215m77, c215m121, c215m115, c215m90, c215m95, c215m106.

4. Method according to any of claims 1 to 3 characterized further in that at least one primer of the primer pair used in step (ii) is selected from the following: AZT 16-bio, AZT 21-bio, AZT 35-bio and AZT 4-bio (SEQ ID NO: 1 to 4).

5. Method according to claim 4 characterized futer in that the set of primers consists of the following 2 primers:
AZT 16-bio (SEQ ID NO: 1) as forward primer and AZT 21-bio (SEQ ID NO: 2) as reverse primer; and/or
AZT 35-bio (SEQ ID NO: 3) as forward primers and AZT 4-bio (SEQ ID NO: 4) as reverse primer.

6. Method according to any of claims 1 to 5 characterized further in that at least two of the mutations of claim 1 are detected simultaneously.

7. Method according to any of claims 1 to 5 characterized farther in that at least three of the mutations of claim 1 are detected simultaneously.

8. Method according to any of claims 1 to 5 characterized farther in that at least four of the mutations of claim 1 are detected simultaneously.

9. Method according to any of claims 1 to 5 characterized farther in that at least five of the mutations of claim 1 are detected simultaneously.

10. Method according to any of claims 1 to 5 characterized farther in that the six mutations K103N/R, V106A/I/L, Y181C/I, Q151M/L, M184V/I, Y188L, G190OA/S/R and T215YIF/D/S/A are detected simultaneously.

11. Method according to any of claims 1 to 10 characterized further in that the mutations associated with anti-HIV drug are induced upon treatment with nucleoside analogues and/or non-nucleoside reverse transcriptase inhibitors.

12. Method according to claim 11 characterized further in that the non-nucleoside reverse transcriptase inhibitor is one of the following: nevirapine, delavirdine or efavirenz.

13. Method according to claim 10 characterized further in that the nucleoside analogue is one of the following: zidovudine, abacavir, 2′,3′-dideoxylnosine, 2′,3′-dideoxyCytidine, (−)-β-L-2′,3′-dideoxy-3′-thioCytidine or 2′,3′-didehydro-3′deoxyThymidine.

14. A probe as defined in any of claims 1 to 13, for use in the genetic detection of the mutations K103N/R, V106A/I/L, Y181C/I, Q151M/L, M184V/I, Y188L, G190A/S/R and/or T215Y/F/D/S/A in the reverse transcriptase of HIV strains present in a biological sample of a patient, said mutations being associated with anti-HIV drug resistance.

15. A composition comprising at least one probe as defined in claim 14.

16. The use of a probe according to claim 14 or a composition of probes according to claim 15 for in vitro detection of mutations associated with anti-HIV drug resistance in a patient.

17. A diagnostic kit for the genetic detection of at least one of the mutations K103N/R, V106A/I/L, Y181C/I, Q151M/L, M184V/I, Y188L, G190A/S/R and/or T215Y/F/D/S/A in the reverse transcriptase of the HIV strains present in a biological sample of a patient, with said mutations being associated with anti-HIV drug resistance, comprising the following components:
(i) when appropriate, a means for releasing, isolating and/or concentrating the polynucleic acids present in said biological sample;
(ii) when appropriate, at least one suitable primer pair;
(iii) at least one probe according to claim 14, possibly fixed to a solid support;
(iv) a hybridization buffer, or components necessary for producing said buffer;
(v) a wash solution, or components necessary for producing said solution;
(vi) when appropriate, a means for detecting the hybrids resulting from the preceding hybridization;
(vii) when appropriate, a means for attaching said probe to a known location on a solid support.

18. A line probe assay for the genetic detection of the K103N/R, V106A/I/L, Y181C/I, M184V/1, Y188L, G19OA/S/R, T215Y/F/D/S/A and/or Q151M/L mutation in the reverse transcriptase of the HIV strains present in a biological sample of a patient, with said mutations being associated with anti-HIV drug resistance, comprising the following components:
(i) when appropriate, a means for releasing, isolating and/or concentrating the polynucleic acids present in a biological sample of the patient;
(ii) when appropriate, at least one suitable primer pair; (iii) at least one probe specifically hybridizing with the K103N/R target sequences shown in FIG. 1 and selected from: c103w62, c103w62b, c103w116, c103w49, c103w115, c103w55, c103w104, c103w52, c103w107, c103w107b, c103w92, c103w97, c103m26, c103m14, c103m22, c103w66, c103w36, c103w36b, 103w65, c103w121, fixed to a solid support; and/or
(iv) at least one probe specifically hybridizing with the V106A/I/L target sequences shown in FIG. 1 and selected from: c103w62, c103w62b, c103w 116, c103w49, c103w115, c103w55, c103w104, c103w52, c103w107, c103w107b, c103w92, c103w97, c103m26, c103m14, c103m22, c103w66, c103w36, c103w36b, c103w65, c103w121, fixed to a solid support; and/or
(v) at least one probe specifically hybridizing with the Y181C/I target sequences shown in FIG. 1 and selected from: c181w3, c181w3b, c181w29, c181w29b, c181w33, c181w38, c181w39, c181w44, c181w53, c181w57, c181w65, c181w65b, c181w65c, c181w69, c181w133, c181w133b, c181w50, c181w97, c181w75, c181m7, c181m7b, c181m14, c181m22, c181m26, c181m144, c181m140, fixed to a solid support; and/or
(vi) at least one probe specifically hybridizing with the Q151M/L target sequences shown in FIG. 1 and selected from: c151w2, c151w51, c151w29, c151w29b, c151w31, c151w52, c151w53, c151m36, c151m48, c151m50, fixed to a solid support; and/or
(vii) at least one probe specifically hybridizing with the M184V/I target sequences shown in FIG. 2 and selected from: c184w85, c184w85b, c184w86, c184w73bis, c184m42bis, c184m42bbis, fixed to a solid support; and/or
(viii) at least one probe specifically hybridizing to the Y188L target sequences shown in FIG. 3 and selected from: c188ww1, c188ww12, c188ww24, c188ww29, c188ww31, c188ww40, c188ww45, c188ww58, c188wm63, c188wm70, c188wm72, c188wm73, c188mw76, 188mw76, c188mw83, c188mw86, 188mw86, c188mm77, 188mm77, c188wm82, c188wm128, c188wm129, fixed to a solid support; and/or
(ix) at least one probe specifically hybridizing to the G190A/S/R target sequences shown in FIG. 3 and selected from: c188ww1, c188ww12, c188ww24, c188ww29, c188ww31, c188ww40, c188ww45, c188ww58, c188wm63, c188wm70, c188wm72, c188wm73, c188mw76, 188mw76, c188mw83, c188mw86, 188mw86, c188mm77, 188mm77, c188wm82, c188wm128, c188wm129, fixed to a solid support; and/or
(x) at least one probe specifically hybridizing with the T215Y/F/D/S/A target sequences shown in FIG. 2 and selected from: c215w145, c215w111, c21 Sm99, c215m139, c215m108, c215m136, c215m84, c215m82, c215m77, c215m121, c215m115, c215m90, c215m95, c215m106, fixed to a solid support;
(xi) a hybridization buffer, or components necessary for producing said buffer;
(xii) a wash solution, or components necessary for producing said solution;
(xiii) when appropriate, a means for detecting the hybrids resulting from the preceding hybridization.

19. A primer for the amplification of the HIV reverse transcriptase, characterized further in that the primer is selected from the following list: AZT 16-bio, AZT 21-bio, AZT 35-bio and AZT 4-bio (SEQ ID NO: 1 to 4).

20. A pair of primers for the amplification of the HIV reverse transcriptase, characterized further in that the set of primers consists of the following two primers:
AZT 16-bio (SEQ ID NO: 1) as forward primer and AZT 21-bio (SEQ ID NO: 2) as reverse primer; and/or
AZT 35-bio (SEQ ID NO: 3) as forward primer and AZT 4-bio (SEQ ID NO: 4) as reverse primer.

21. An isolated single or double-stranded polynucleotide or polyribonucleotide comprising the target sequence defined in FIGS. 1, 2 and/or 3 or part thereof.

22. An isolated single or double-stranded polynucleotide or polyribonucleotide comprising at least one of the following target sequences: nucleotide positions 304 to 315, 303 to 316, 302 to 317, 307 to 318, 306 to 319, 305 to 320, 445 to 453, 444 to 454, 443 to 455, 449 to 455, 448 to 456, 447 to 457, 535 to 543, 534 to 544, 533 to 545, 538 to 546, 537 to 547, 536 to 548, 541 to 549, 540 to 550, 539 to 551, 541 to 566, 544 to 555, 543 to 556, 542 to 557, 550 to 561, 549 to 562, 548 to 563, 562 to 570, 561 to 571, 560 to 572, 637 to 645, 636 to 646, 635 to 647, 640 to 648, 639 to 649, 638 to 650, 643 to 651, 642 to 652, 641 to 653 and/or 634 to 656 of the HIV reverse transcriptase gene.

23. A probe selectively hybridizing to a target sequence according any of claims 21 and/or 22.

24. A method for determination of the nucleic acid sequence of a target sequence of any of claims 21 and/or 22.

25. A method according to claim 24, wherein at least the amino acids encoded by the codons located between nucleotide positions 307 and 309, 316 and 318, 451 and 453, 541 and 543, 550 and 552, 562 and 564, 568 and 570 and/or 643 and 645 are determined.

26. A method according to claims 24 or 25 for the determination of viral mutations and/or polymorphisms known to be associated with resistance.

27. A set of at least two probes selectively hybridizing to at least two target sequences according to any of claims 21 and/or 22.

28. A nucleic acid comprising a nucleotide sequence selected from the group consisting of: c103w66, c103w107b, c103w116, c103w121, c103m22, c103m26, c181w65c, c181w69, c181w75, c181w133, c181m26, c184w85b, c188mw76, 188mw76, c188mm77, 188mm77, cl 88mw86, 188mw86, the complement thereof or a fragment thereof, wherein the fragment contains at least one polymorphic nucleotide.

29. A nucleic acid comprising a nucleotide sequence selected from the group consisting of: SEQ ID NO: 833, SEQ ID NO: 835, SEQ ID NO: 837, SEQ ID NO: 839, SEQ ID NO: 841, SEQ ID NO: 843, SEQ ID NO: 845, SEQ ID NO: 847, SEQ ID NO: 849, SEQ ID NO: 851, SEQ ID NO: 853, SEQ ID NO: 855, SEQ ID NO: 857, SEQ ID NO: 858, SEQ ID NO: 859, SEQ ID NO: 860, SEQ ID NO: 862, SEQ ID NO: 864, the complement thereof or a fragment thereof, wherein the fragment contains at least one polymorphic nucleotide.

30. Use of a nucleic acid according to claim 28 or 29 in a method for detection of mutations and/or polymorphisms in the HIV RT gene.

31. Method for detection of mutations and/or polymorphisms in the HIV RT gene, said method comprising the step of detecting the presence of a nucleic acid according to claim 28 or 29, the complement thereof or part thereof.

32. Method according to claim 31, further characterized in that a sequencing reaction is used.

33. Method according to claim 31, further characterized in that a hybridization reaction with at least one nucleic acid according to claim 28 or 29 is used.
